(19) United States
(12) Patent Application Publication
(10) Pub. No.: US 2025/0049678 A1
(43) Pub. Date: Feb. 13, 2025

(54) COMPOUNDS ATTACHABLE TO SKIN

(71) Applicant: Inkbox Ink Incorporated, Toronto (CA)

(72) Inventors: Christopher B. CAPUTO, Toronto (CA); Sanjay MANHAS, Toronto (CA)

(73) Assignee: Inkbox Ink Incorporated, Toronto, ON (CA)

(21) Appl. No.: 18/717,276

(22) PCT Filed: Dec. 7, 2022
(86) PCT No.: PCT/CA2022/051783

§ 371 Date: Jun. 6, 2024

Related U.S. Application Data
(60) Provisional application No. 63/287,069, filed on Dec. 7, 2021.

(51) Int. Cl. A61K 8/49; A61Q 17/04; C07D 311/94; C07D 493/06

(52) U.S. Cl. CPC A61K 8/498; A61Q 17/04; C07D 311/94; C07D 493/06; A61K 2800/43

(57) ABSTRACT

The present application provides compounds which are attachable to skin, in particular hydrogenated genipin compounds and de-glycosylated oleuropein compounds, which are useful, for example, as colorants and sunscreens.

### TECHNICAL FIELD

This disclosure relates to compounds which are attachable to skin, such as hydrogenated genipin and oleuropein derivatives. In one aspect, the disclosure provides conjugates of organic dye molecules and UV-absorbing molecules to the hydrogenated genipin and deglycosylated oleuropein derivatives. The dye conjugates are useful, for example, as colorants in semi-permanent tattoos, while the UV-absorbing conjugates are useful, for example, in semi-permanent sunscreen compositions.

### BACKGROUND

Cancer is one of the leading causes of death in contemporary society. The numbers of new cancer cases and deaths is increasing each year. Currently, cancer incidence is nearly 450 cases of cancer per 100,000 men and women per year, while cancer mortality is nearly 71 cancer deaths per 100,000 men and women per year. The socioeconomic burden of melanoma in particular is constantly increasing. Overall, melanoma incidence increasers at a rate of 3% per year, making it one of the fastest growing cancers worldwide. It is widely recognized that excessive exposure to UV sunlight is the major underlying cause of melanoma, and that currently available sunscreen provide only a limited protection.

Temporary or semi-permanent tattoo or body inks have been used throughout human history to decorate the body. Generally, temporary or semi-permanent tattoos are transferred to the skin through the direct exposure of the skin to tattoo ink over a designated incubation period. The quality of the transferred image and its duration on the skin can depend on the ink distribution profile in the outer layer of the skin called the stratum corneum. Current manufacturing processes for pre-fabricated tattoo designs include the utilization of flexographic or gravure printing, stencils, and inkjet printers. However, due to the barrier properties of the stratum corneum, many of the current methods require moderate oversight by the end user during the tattoo application process to ensure adequate ink delivery to the skin such that the tattoo ink develops properly.

### SUMMARY

In its broadest disclosure, the present disclosure relates to a compound of formula (I),

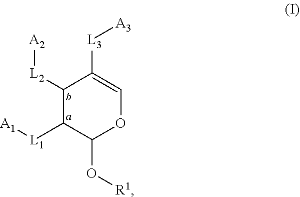

wherein A₁, A₂ and A₃ independently from each other represent
a) a color-imparting moiety,
b) a UVA and/or UVB absorbing moiety, or
c) H, hydroxyl, amino, a halogen or a C₁-C₃₀ moiety.

R¹ in the above formula represents hydrogen or a protective group hydrolysable under physiological conditions after application of the compound (e.g. in a topical composition) onto skin. L₁, L₂ and L₃ independently from each other represent a linker group or are absent. Furthermore, L₁-A₁ and L₂-A₂ do not represent a moiety comprising an unsaturated C—C or C—N bond in alpha-position to the carbon atom marked “a” and “b”, respectively.

The present disclosure is based, at least in part, on the realization that compounds of the aforementioned formula, when applied to skin, provide a strong covalent bonding of the compound with an amino acid (e.g., lysine) within a protein (e.g., collagen) of the skin. More specifically, the cyclic hemiacetal (i.e. when R¹ is H), when applied to or generated in-situ on the skin, reacts with skin proteins under formation of a 1,4-dihydropyridine derivative as shown in the below reaction scheme:

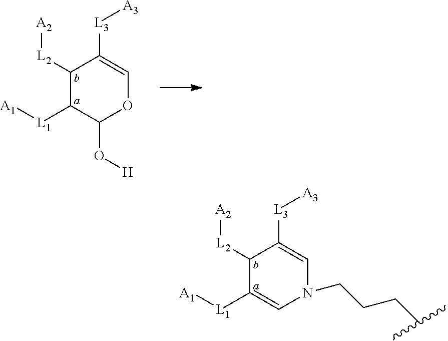

The present disclosure is based, at least in part, on the further realization that the above 1,4-dihydropyridine moiety is colorless (or nearly colorless and, thus, not perceivable on the skin), in contrast to the similar compound genipin which provides a blue color on the skin. Genipin is the compound methyl (1R,4aS,7aS)-1-hydroxy-7-(hydroxymethyl)-1,4a,5,7a-tetrahydrocyclopenta[c]pyran-4-carboxylate (CAS RN. 6902-77-8) having the following structure:

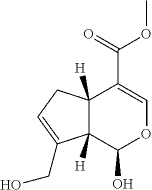 [SMILES: COC(=O)C1=CO[C@@H](O)[C@@H]2C(CO)=CC[C@H]12]

This lack of absorption in the visible range can be attributed to the lack of double bonds capable of forming a conjugated system with the double bonds of the 1,4-dihydropyridine moiety. For this reason, L₁-A₁ and L₂-A₂ are defined in the above formula to not represent a moiety comprising an unsaturated C—C or C—N bond in alpha-position to the carbon atom marked “a” and “b”, respectively, since this definition excludes the possibility of the formation of such conjugated systems.

The compound of the above formula may also be present as its tautomer and/or as a pharmaceutically acceptable salt.

Although not limited thereto, the present disclosure will in the following be discussed primarily with reference to hydrogenated genipin derivatives or de-glycosylated oleuropein derivatives. These compounds are embodiments of the above formula. However, given the robust and very reliable linking chemistry of the cyclic hemiacetal to skin, the skilled person will readily realize that other compounds can be equally used in accordance with the following disclosure.

An example of a synthetic and conceptual approach on basis of hydrogenated genipin is schematically shown in FIG. 1. Referring to FIG. 1, hydrogenation of genipin attenuates its original color to yield an anchor molecule. A moiety having the desired property (e.g., a dye molecule having the desired color or a UV-absorbing molecule) may then be attached to the hydrogenated anchor either through the alcohol or the ester functionality. When the conjugate is applied to the skin, the anchor molecule covalently binds with a side chain of an amino acid (e.g., lysine) within the collagen or keratin, thereby attaching the moiety to the skin.

When the moiety having the desired property is a dye, advantageously, application of the conjugate to the skin and covalent bonding of the anchor molecule to the skin protein do not alter the intensity and the visible color of the original dye moiety. What is more, after application to the skin, the anchor molecule itself remains colorless, allowing the dye to retain its original intensity and the desired visible color for its duration on the skin (e.g., until natural skin desquamation).

When the moiety having the desired property is a UV-absorbing sunscreen molecule, advantageously, application of the conjugate to the skin and covalent bonding of the anchor molecule to the skin protein affords long-term protection of the skin against ultraviolet (UV) rays. For example, the sunscreen protection is provided for as long as the UV-absorbing molecule remains covalently attached to the skin through the anchor molecule (e.g., until natural skin desquamation). What is more, because the anchor molecule remains colorless after application, the skin does not change its color after the sunscreen application, which is both esthetically and cosmetically pleasing.

However, it was also found that hydrogenated genipin derivatives not conjugated to a dedicated UV-absorbing moiety provide some UVA/B-absorbing capability, presumably due to the presence of the 1,4-dihydropyridine moiety. Therefore, compounds of the present disclosure not carrying a dedicated UV-absorbing moiety, i.e. compounds of the above formula wherein none of A₁, A₂ and A₃ represents a UVA and/or UVB absorbing moiety, are also useful as sunscreen agents.

Accordingly, the present disclosure further relates to the following aspects:

In a first aspect, the present disclosure relates to a topical composition comprising a compound of formula (I),

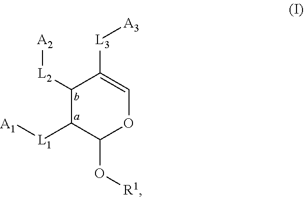

or a tautomer and/or a pharmaceutically acceptable salt thereof, wherein A₁, A₂ and A₃ independently from each other represent a) a color-imparting moiety; b) a UVA and/or UVB absorbing moiety; or c) a C₁-C₃₀ moiety, H, hydroxyl, amino, or a halogen; wherein R¹ represents hydrogen or a protective group hydrolysable under physiological conditions after application of the topical composition onto skin; wherein L₁, L₂ and L₃ independently from each other represents a linker group or are absent; and wherein L₁-A₁ and L₂-A₂ do not represent a moiety comprising an unsaturated C—C or C—N bond in alpha-position to the carbon atom marked “a” and “b”, respectively; and an excipient suitable for topical administration.

In a second aspect, there is provided a (non-therapeutic) method of semi-permanently tattooing skin of a subject or a (non-therapeutic) method of protecting skin of a subject from UV sunlight, the method comprising applying to the skin of the subject the topical composition according to the aforementioned first aspect.

Embodiments according to the first and second aspect will be discussed below in the detailed description.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 is a schematic showing how hydrogenation of genipin attenuates the formation of color after reaction with amino acids (e.g., in the skin). A moiety having a desired property (e.g., a dye molecule having desired color or a UV-absorbing molecule) may be covalently attached to the anchor molecule, in which case the moiety retains its original properties after the anchor molecule binds to the skin.

FIG. 2 is a line plot showing that compound 1-lysine conjugate has a strong UV-light absorption and no visible color.

FIG. 3A is a line plot showing that compound 2-lysine conjugate has a strong UV-light absorption and no visible color.

FIG. 3B is a line plot showing that compound 6-lysine conjugate has a strong UV-light absorption and no visible color.

FIG. 3C is a line plot showing that compound 8-lysine conjugate has a strong UV-light absorption and no visible color.

FIG. 3D is a line plot showing that the azo-dye-anchor molecule retains its original color after the molecule binds to lysine in the solution.

FIG. 4 is an image showing that exemplified compounds absorb UV light on the skin substrate after 2 and 4 hours after application.

FIG. 5 is an image showing that exemplified compounds absorb UV light on the skin substrate after 24 and 26 hours after application, even after the substrate is washed for 2 hours and scrubbed.

FIG. 6 is a line plot showing that lysine conjugate of the hydrogenated genipin-based exemplified compound has a strong UV-light absorption and no visible color.

FIG. 7 is a line plot showing that lysine conjugate of the oleuropein-based exemplified compound has a strong UV-light absorption and no visible color.

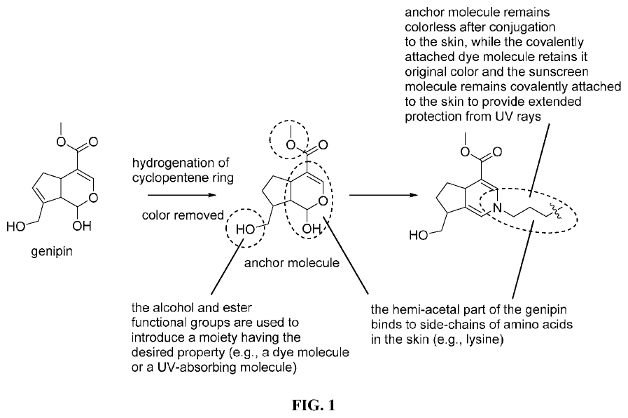

### DETAILED DESCRIPTION

The present disclosure relates to a compound of formula (I), in particular comprised in a topical formulation together with an excipient suitable for topical administration:

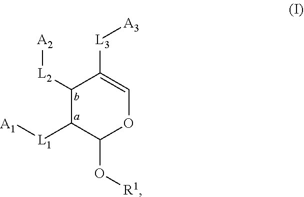

or a tautomer and/or a pharmaceutically acceptable salt thereof, wherein A₁, A₂ and A₃ independently from each other represent a) a color-imparting moiety, b) a UVA and/or UVB absorbing moiety, or c) C₁-C₃₀ moiety, H, hydroxyl, amino, or a halogen; wherein R¹ represents hydrogen or a protective group hydrolysable under physiological conditions after application of the topical composition onto skin, wherein L₁, L₂ and L₃ independently from each other represents a linker group or are absent, and wherein L₁-A₁ and L₂-A₂ do not represent a moiety comprising an unsaturated C—C or C—N bond in alpha-position to the carbon atom marked “a” and “b”, respectively.

In some embodiments, at least one of A₁, A₂ and A₃ is a moiety from the above group a) or b). It may be particularly advantageous that two or more of A₁, A₂ and A₃ represent a moiety from above the group a) or that or two or more of A₁, A₂ and A₃ represent a moiety from the above group b) since the combination of color-imparting moieties may help in realizing novel and vibrant tattooing dyes and the combination of UV-absorbing moieties may help in optimizing the UV absorbance over the entire UVA and UVB spectrum.

In some embodiments, the color-imparting moiety has at least one absorption peak within the wavelength range of 380 to 790 nm. Determining the presence of such an absorption peak is not particularly limited and can be done experimentally by e.g. vis-absorption spectroscopy or by computational methods or by subtracting the peaks from a known library compound from the measured vis absorption.

In some embodiments, the UVA and/or UVB absorbing moiety has at least one absorption peak within the wavelength range of 280 to 379 nm. Determining the presence of such an absorption peak is not particularly limited and can be done experimentally by e.g. UV-absorption spectroscopy or by computational methods or by subtracting the peaks from a known library compound from the measured UV absorption.

In some embodiments, the topical composition according to any preceding embodiment, wherein one or more of A₁, A₂ and A₃ represent a color-imparting moiety, wherein said moiety is: a) an optionally substituted (hetero)aromatic moiety comprising, in combination with its optional substituents, 4 to 40 carbon atoms, or b) an optionally substituted conjugated moiety comprising, in combination with its optional substituents, 6 to 40 carbon atoms and at least 3 conjugated C—C double bonds.

In some embodiments, one or more of A₁, A₂ and A₃ represents a UVA and/or UVB absorbing moiety, wherein said moiety is: a) an optionally substituted (hetero)aromatic moiety comprising, in combination with its optional substituents, 4 to 36 carbon atoms, or b) an optionally substituted conjugated moiety comprising, in combination with its optional substituents, 4 to 36 carbon atoms, and at least 2 conjugated C—C double bonds.

In some embodiments, one or more of A₁, A₂ and A₃ comprises a chromophore (which is also interchangeably called dye moiety elsewhere in this disclosure) selected from an azo group; a diazo group; a diphenylamine group; a nitroarylamine group; an azine group; an oxazine group; an acridine group; an indoline group; a sulfur dye group, in particular a thiazine group, a thiazole group, a thiazone group, a thianthrene group, or a phenothiazonethioanthrone group; a quinoid or quinone group; an anthraquinoid or anthraquinone group; a xanthene group; a naphthostyryl group; a diaryl methyl or triarylmethyl group; a benzodifuranone-based group; a formazan group; a phthalocyanine group; or a metal complex.

The exact chemical structure of the chromophore is not particularly limited and can be chemically very diverse since the linking of the hemi-acetal ring to skin is reliable irrespective of the precise nature of the chromophore. As such, suitable examples of a color-imparting moiety include the following list of exemplary moieties:

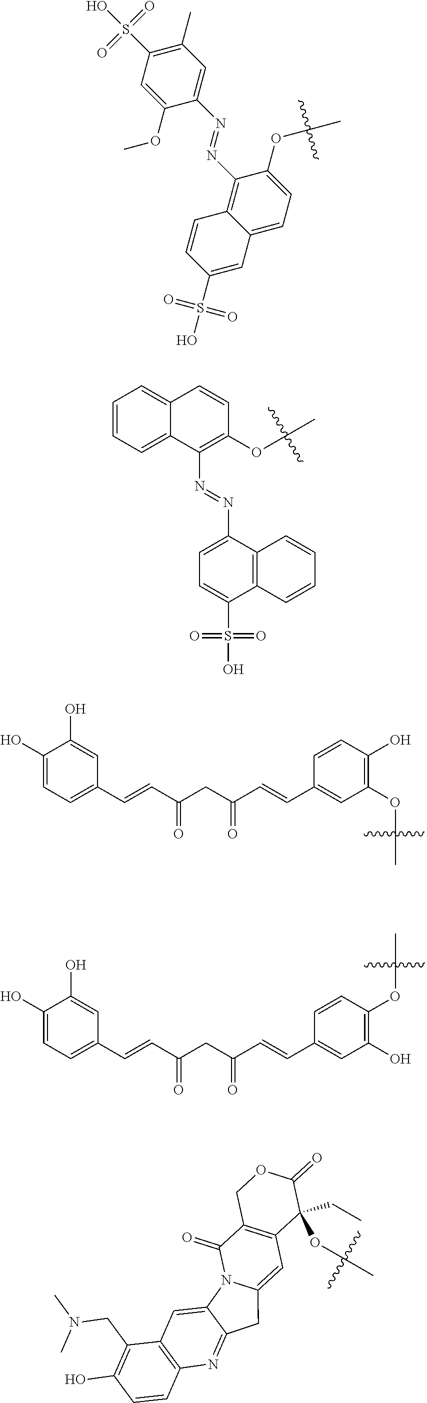

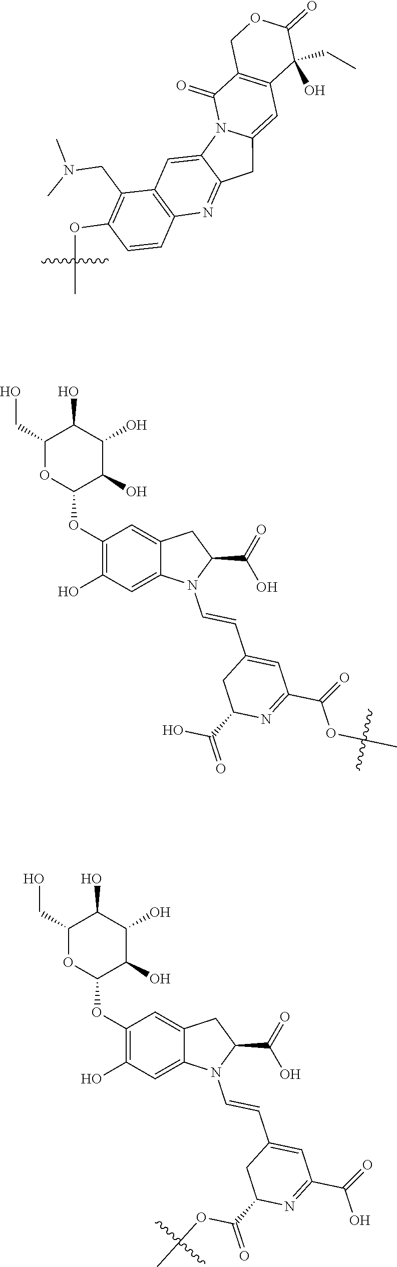 [SMILES: *Oc1ccc2nc3c(cc2c1CN(C)C)-n1c(cc2c(c1=O)COC(=O)[C@]2(O)CC)C3 ; *OC(=O)C1=N[C@H](C(=O)O)CC(/C=C/N2c3cc(O)c(O[C@@H]4O[C@H](CO)[C@@H](O)[C@H](O)[C@H]4O)cc3C[C@H]2C(=O)O)=C1 ; *OC(=O)[C@@H]1CC(/C=C/N2c3cc(O)c(O[C@@H]4O[C@H](CO)[C@@H](O)[C@H](O)[C@H]4O)cc3C[C@H]2C(=O)O)=CC(C(=O)O)=N1]

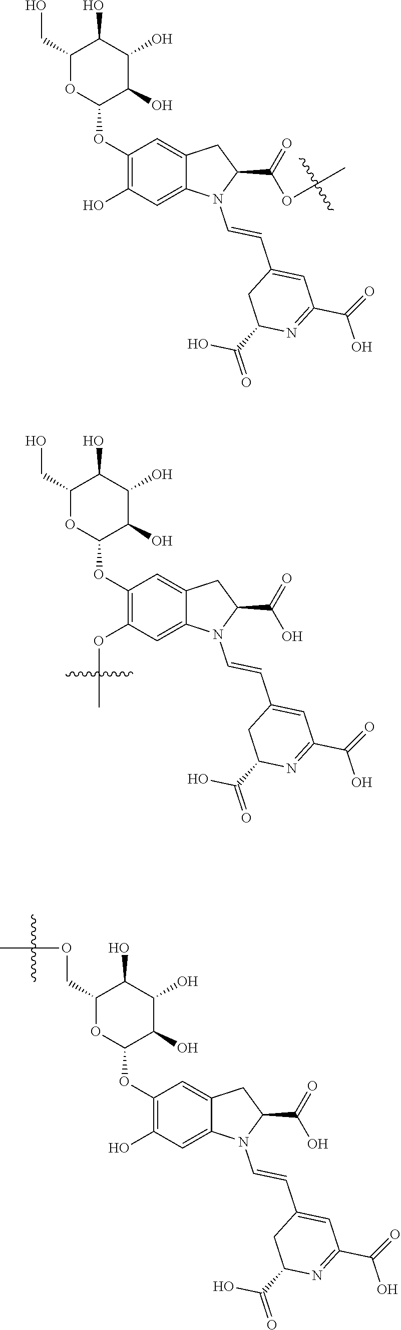 [SMILES: *OC(=O)[C@@H]1Cc2cc(O[C@@H]3O[C@H](CO)[C@@H](O)[C@H](O)[C@H]3O)c(O)cc2N1/C=C/C1=CC(C(=O)O)=N[C@H](C(=O)O)C1 ; *Oc1cc2c(cc1O[C@@H]1O[C@H](CO)[C@@H](O)[C@H](O)[C@H]1O)C[C@@H](C(=O)O)N2/C=C/C1=CC(C(=O)O)=N[C@H](C(=O)O)C1 ; *OC[C@H]1O[C@@H](Oc2cc3c(cc2O)N(/C=C/C2=CC(C(=O)O)=N[C@H](C(=O)O)C2)[C@H](C(=O)O)C3)[C@H](O)[C@@H](O)[C@@H]1O]

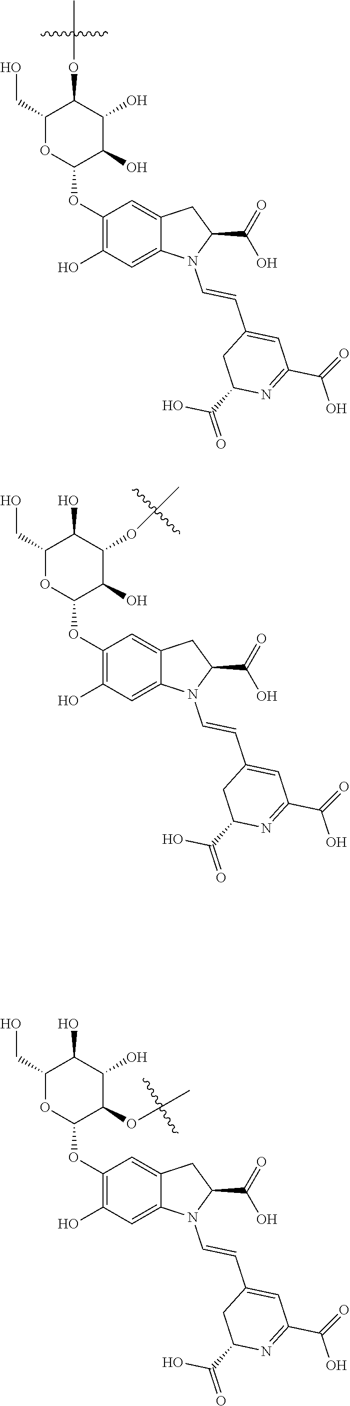 [SMILES: *O[C@H]1[C@H](O)[C@@H](O)[C@H](Oc2cc3c(cc2O)N(/C=C/C2=CC(C(=O)O)=N[C@H](C(=O)O)C2)[C@H](C(=O)O)C3)O[C@@H]1CO ; *O[C@@H]1[C@@H](O)[C@H](Oc2cc3c(cc2O)N(/C=C/C2=CC(C(=O)O)=N[C@H](C(=O)O)C2)[C@H](C(=O)O)C3)O[C@H](CO)[C@H]1O ; *O[C@H]1[C@H](Oc2cc3c(cc2O)N(/C=C/C2=CC(C(=O)O)=N[C@H](C(=O)O)C2)[C@H](C(=O)O)C3)O[C@H](CO)[C@@H](O)[C@@H]1O]

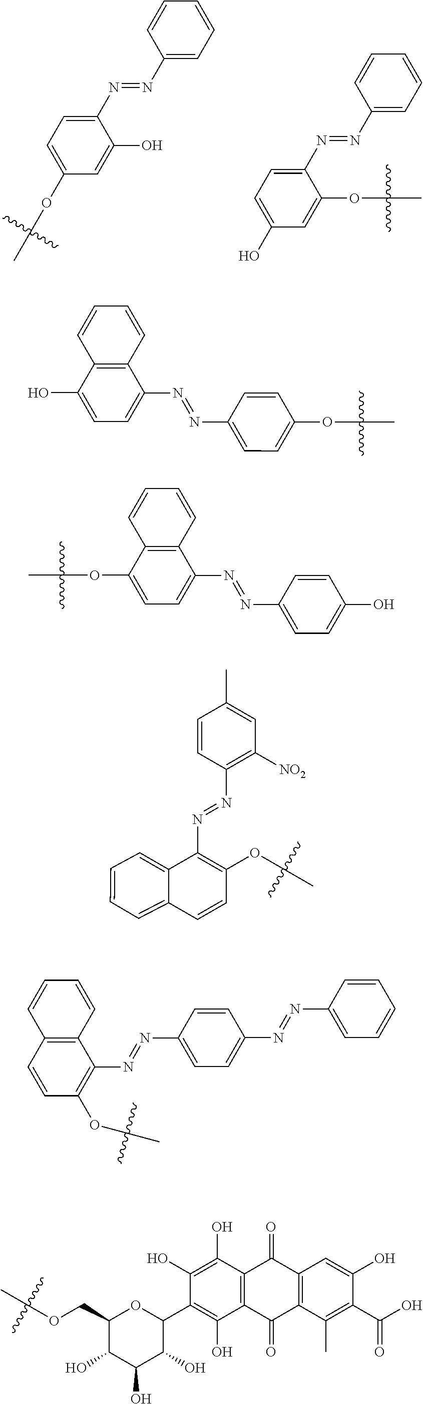

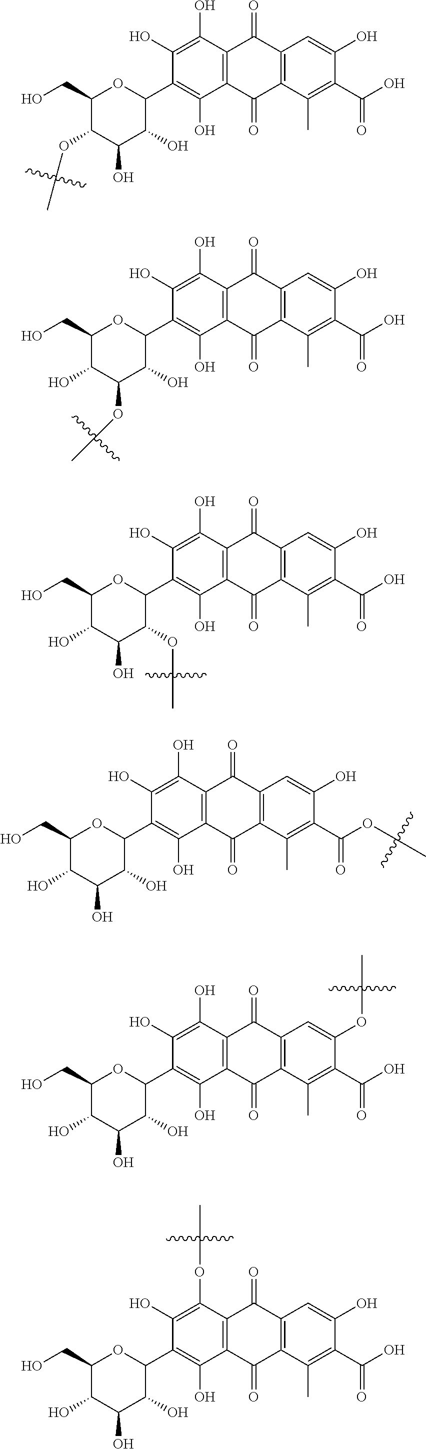

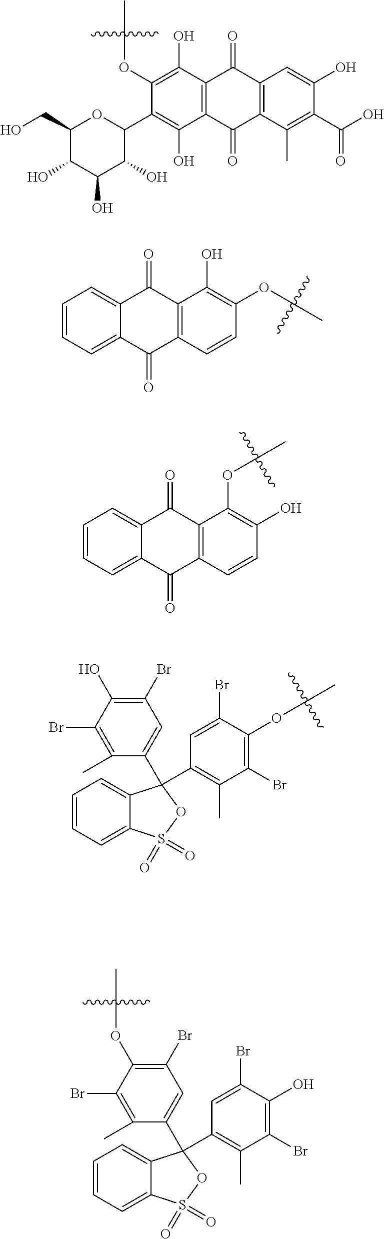 [SMILES: *Oc1c(O)c2c(c(O)c1C1O[C@H](CO)[C@@H](O)[C@H](O)[C@H]1O)C(=O)c1c(cc(O)c(C(=O)O)c1C)C2=O ; *Oc1ccc2c(c1O)C(=O)c1ccccc1C2=O ; *Oc1c(O)ccc2c1C(=O)c1ccccc1C2=O ; *Oc1c(Br)cc(C2(c3cc(Br)c(O)c(Br)c3C)OS(=O)(=O)c3ccccc32)c(C)c1Br ; *Oc1c(Br)cc(C2(c3cc(Br)c(O)c(Br)c3C)OS(=O)(=O)c3ccccc32)c(C)c1Br]

In some embodiments, one or more of A₁, A₂ and A₃ comprises a UVA and/or UVB absorbing moiety and being selected/derived from benzophenone group, a benzotriazole group, a benzone group, salicylic acid or a salicylic acid derivative, a benzocaine group, an esculin or an esculin derivative, a ferulic acid or a ferulic acid derivative, octinoxat or an octinoxat derivative, or octocrylene or an octocrylene derivative.

The exact chemical structure of the UVA and/or UVB absorbing moiety is not particularly limited and can be chemically very diverse since the linking of the hemi-acetal ring to skin is reliable irrespective of the precise nature of the UVA and/or UVB absorbing moiety. As such, suitable examples of a UVA and/or UVB absorbing moiety include the following list of exemplary moieties:

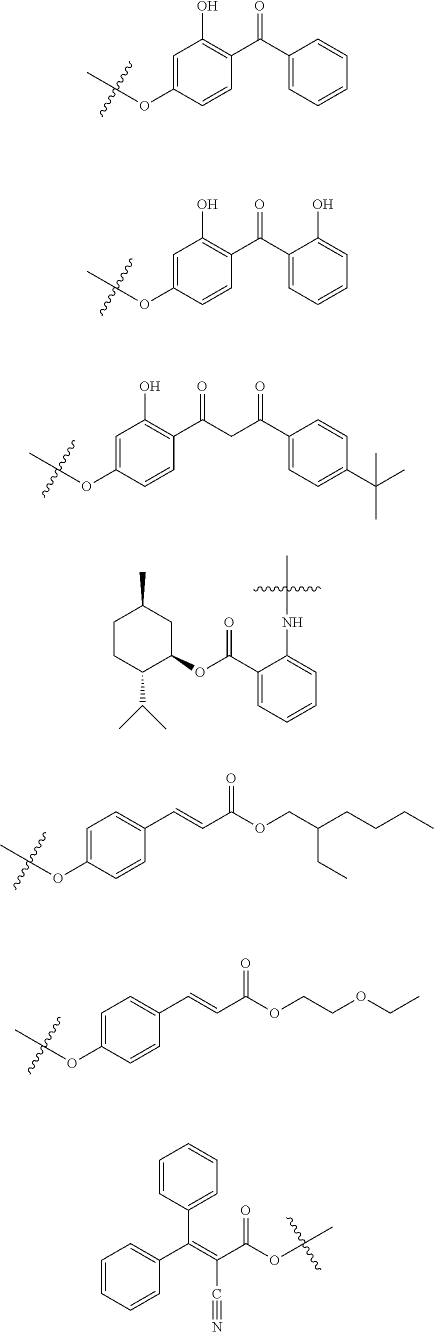

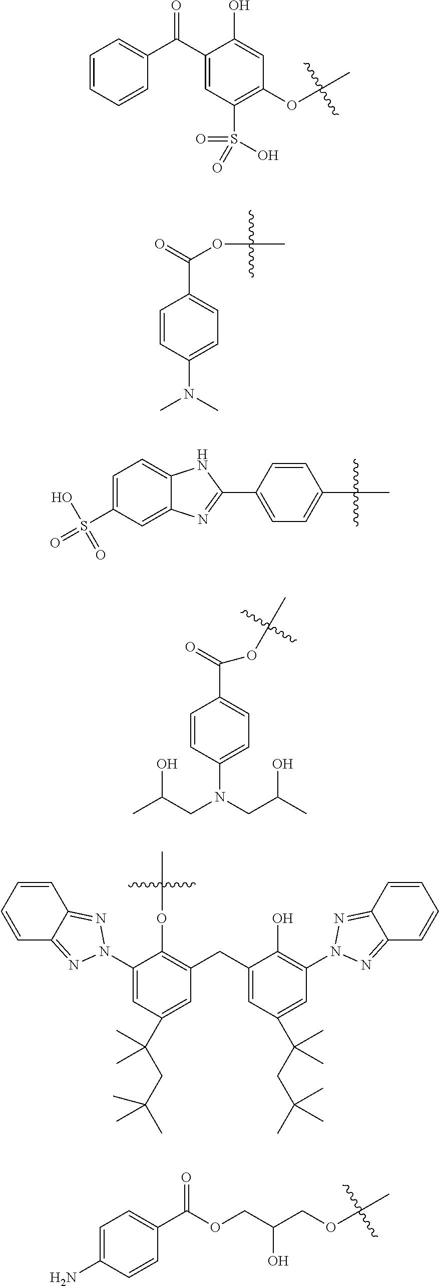 [SMILES: *Oc1cc(O)c(C(=O)c2ccccc2)cc1S(=O)(=O)O ; *OC(=O)c1ccc(N(C)C)cc1 ; *c1ccc(-c2nc3cc(S(=O)(=O)O)ccc3[nH]2)cc1 ; *OC(=O)c1ccc(N(CC(C)O)CC(C)O)cc1 ; *Oc1c(Cc2cc(C(C)(C)CC(C)(C)C)cc(-n3nc4ccccc4n3)c2O)cc(C(C)(C)CC(C)(C)C)cc1-n1nc2ccccc2n1 ; *OCC(O)COC(=O)c1ccc(N)cc1]

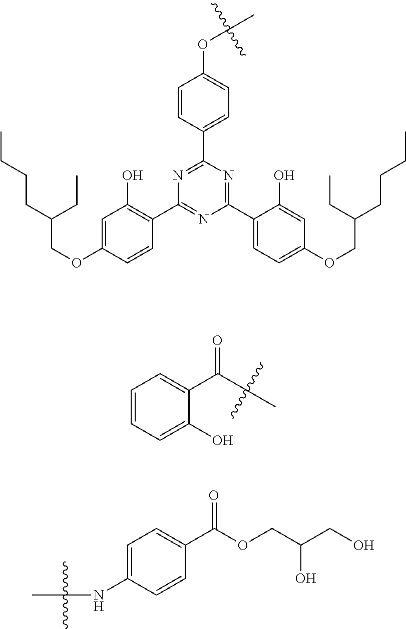 [SMILES: *Oc1ccc(-c2nc(-c3ccc(OCC(CC)CCCC)cc3O)nc(-c3ccc(OCC(CC)CCCC)cc3O)n2)cc1 ; *C(=O)c1ccccc1O ; *Nc1ccc(C(=O)OCC(O)CO)cc1]

When referring to protective groups hydrolysable under physiological conditions after application of the topical composition onto the skin here and elswhere in the dislcosure: Such groups are readily known to the skilled person and include, in particular acyl moieties such as optionally substituted acyl moieties comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms. Specific examples include C₁-C₁₈ acyl moieties, more specifically C₁-C₁₂ acyl moieties and in particular C₁-C₁₂ acyl moieties. When referring in this context to “physiological conditions” it should be understood that this in particular refers to the pH conditions encountered at the locus where the compound of formula (I) is supposed to bind to proteins in e.g. the stratum corneum and/or the epidermis of the (mammalian, in particular human) subject to be treated. Additionally or alternatively, the term “protective groups hydrolysable under physiological conditions after application of the topical composition” refers to a group which is hydrolysed when placing 0.1 mol/l of the compound of formula (I) in aqueous solution having a pH of about 5 and further containing 0.1 mol/l lysine at 37° C. for 2 hours, wherein the group in question qualifies as “protective groups hydrolysable under physiological conditions after application of the topical composition” if a notable amount of a 1,4-dihydropyridine derivative (e.g. conversion yield greater than 10 mol %) is formed. Additionally or alternatively, a compound of formula (I) contains a “protective groups hydrolysable under physiological conditions after application of the topical composition” if the compound (or a topical composition containing the compound) cannot be comprehensively washed off from (explanted) porcine skin by water, soap, and/or isopropanol after incubating the compound (or the topical composition comprising the compound) on the porcine skin at e.g. 37° C. for e.g. 2 hours. This type of test was used in the experimental section and revealed that, for instance, that glycosylated oleuropein derivatives are not hydrolysable under these conditions and, thus, do not fall in the ambit of formula (I).

In some embodiments, L₁ and L₂, if present, independently from each other represent optionally substituted hydrocarbon moieties each comprising, in combination with their optional substituents, 1 to 8 carbon atoms; or wherein L₁ and L₂ form a 5-, 6-, 7- or 8-membered ring, in particular a cyclopentyl, a cyclohexyl, a pyrrolidinyl, a piperidinyl, a tetrahydrofuranyl or a tetrahydropyranyl.

In some embodiments, L₁ and L₂ form an optionally substituted 5- or 6-membered ring, in particular a cyclopentyl or cyclohexyl, to which A₁ and A₂ are attached, optionally via a group selected from: —O—, —S—, —C(O)—, —CO₂—, —O—C(O)—, —NH—C(O)—, —C(O)—NH—, —(CH₂)₁₋₄—, —(CH₂)₁₋₄—O— and —O—(CH₂)₁₋₄—.

In some embodiments, the C₁-C₃₀ moiety comprises 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms.

In some embodiments, it may be particularly advantageous that the C₁-C₃₀ moiety is selected from a saturated or unsaturated, cyclic or acrylic (hetero)alkyl comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms. Additionally or alternatively, the C₁-C₃₀ moiety may be bound to L₁, L₂ and L₃, respectively, via a carbon atom, an oxygen atom, a nitrogen atom or a sulfur atom.

In some embodiments, A₃ represents a C₁-C₁₆ moiety selected from a saturated or unsaturated, cyclic or acrylic (hetero)alkyl comprising 1 to 16, more specifically 1 to 12, and in particular 1 to 8, carbon atoms; 0 to 6, more specifically 0 to 4, and in particular 0 to 3 oxygen atoms; 0 to 6, more specifically 0 to 4, and in particular 0 to 3 nitrogen atoms; 0 to 6, more specifically 0 to 4, and in particular 0 to 3 sulfur atoms; and 0 to 6, more specifically 0 to 4, and in particular 0 to 3 halogen atoms. It may be further advantageous that A₃ represents a C₁-C₁₆ moiety selected from a carboxylic acid or a salt thereof; a carboxylic acid ester; or a ketone.

In some embodiments, L₁ and L₂, if present, independently from each other represent optionally substituted hydrocarbon moieties each comprising, in combination with their optional substituents, 1 to 8 carbon atoms; or wherein L₁ and L₂ form a 5-, 6-, 7- or 8-membered ring, in particular a cyclopentyl, a cyclohexyl, a pyrrolidinyl, a piperidinyl, a tetrahydrofuranyl or a tetrahydropyranyl.

In some embodiments, L₁ and OR¹, together with the carbon atoms to which they are attached, form a 5- or 6-membered lactone.

In some embodiments, R₁ represents hydrogen or a C₁₋₆ acyl.

In some embodiments, the compound of formula (I) is comprised in a topical composition and the topical composition is in the form of an aerosol spray, a cream, an emulsion, a solid, a liquid, a dispersion, a foam, an oil, a gel, a hydrogel, a lotion, a mousse, an ointment, a patch for transferring the composition onto skin, a substrate carrier comprising the topical composition, a pump spray comprising the topical composition, or a stick comprising the topical composition. Examples of a patch include a patch or adhesive label or thin foil on which the composition is coated or printed. Examples of a carrier includes a non-woven material or a hydrogel in which the composition is impregnated.

In some embodiments, the excipient suitable for topical administration comprises one or more excipients selected from water, ethanol, isopropanol, n-propanol, ethylene glycol, diethylene glycol, a propylene glycol, and glycerol.

In some embodiments, the topical composition has (or provides when applied onto skin) a sun protection factor (SPF) of at least 2, more specifically at least 4, and in particular at least 8. The SPF value represents the ratio of time for sunburn by UV with and without the protection of the product. For example, in the case of skin irradiated by UV, using SPF8 it takes 8 times longer to obtain the same amount of sunburn to the skin than without using the product. The test methods for SPF determinations are not particularly limited and can be done as approved by either the FDA or European regulatory authorities. The SPF value may also be determined in accordance with ISO-24442 or ISO-24444. The SPF value may alternatively also be determined as indicated Application Note 130-UV-0041 from the company Jasco Inc., MD (USA), utilizing their SPF/PA evaluation system mentioned in said application note.

In some embodiments, there is provided a compound of formula (II) or the topical composition comprising it wherein the compound of formula (I) is a compound of formula (II),

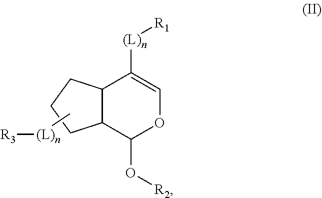

or a tautomer and/or a pharmaceutically acceptable salt thereof, wherein: R¹ and R³ independently from each other represent H; a C₁-C₃₀ moiety comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms; —SO₃H; —NO₂; —NH₂; —OH; —SH; or halogen, in particular Cl, Br or F; and, in case of R³, oxo; R² is selected from H or a protective group hydrolysable under physiological conditions after application in a topical composition onto the skin; and optionally wherein (L)ₙR³ and OR², together with the carbon atoms to which they are attached, form a tetrahydrofuran ring which is optionally substituted with OR^{a1} or oxo; n is 0 or is an integer selected from 1 to 10, wherein, if n=0, (L)ₙ represents a bond;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy; and each R^{N} is independently selected from H and C₁₋₃ alkyl.

In some embodiments, the compound of formula (II) is not any one of the following compounds:

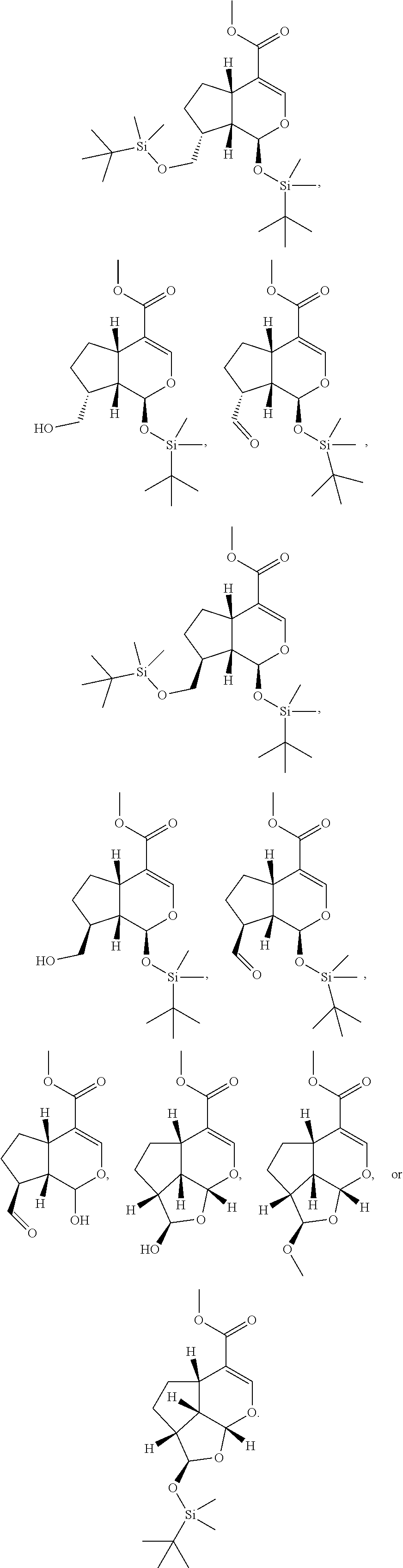

In some embodiments and referring to the compound of formula (II), the R¹ and/or R³ represent said C₁-C₃₀ moiety which further is a color-imparting moiety, in particular a color-imparting moiety having at least one absorption peak within the wavelength range of 380 to 790 nm.

In some embodiments and referring to the compound of formula (II), R¹ and/or R³ represent said C₁-C₃₀ moiety which further is a UVA and/or UVB absorbing moiety, in particular a UVA and/or UVB absorbing moiety having at least one absorption peak within the wavelength range of 280 to 379 nm.

In some embodiments and referring to the compound of formula (II), the C₁-C₃₀ moiety of R¹ and/or R³ is selected from: a) an optionally substituted saturated or unsaturated, cyclic or acyclic hydrocarbon comprising, together with its optional substituents, 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms; and b) an optionally substituted (hetero)aromatic moiety comprising, together with its optional substituents, 4 to 30, more specifically 4 to 16, and in particular 4 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms.

In some embodiments, the compound of formula (II) is a compound of formula (III),

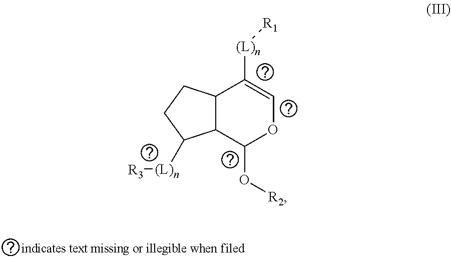

wherein R¹, R², R³, L and n are as defined above for the compound of formula (II).

In some embodiments and referring to the compound of formula (II) and (III), the C₁-C₃₀ moiety of R³ is attached to (L)ₙ or the cyclopentyl ring of formulae (II) or (III) via a carbon atom, an oxygen atom, a nitrogen atom or a sulfur atom. In some embodiments and referring to the compound of formulae (II) and (III), the C₁-C₃₀ moiety of R¹ is attached to (L)ₙ or to the 3,4-dihydro-2H-pyran ring of formulae (II) or (III) via a carbon atom, an oxygen atom, a nitrogen atom or a sulfur atom.

In some embodiments and referring to the compound of formula (II) and (III), (L)ₙR³ represents Rₐ, O—R^{a}, SR^{a}, (CH₂)₁₋₄—O—R^{a}, C(O)R^{a}, CO₂R^{a}, O—C(O)R^{a}, NR^{a}₂, NHR^{a}, NHC(O)R^{a}, NR^{a}C(O)R^{a}, or oxo, wherein R^{a} independently from each other represents a C₁-C₁₈ moiety comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms. It may be particularly advantageous that R^{a}, independently from each other, represents C₁₋₁₈ alkyl, C₂₋₁₈ alkenylene, C₂₋₁₈ alkynylene, C₆₋₁₈ aryl, C₄₋₁₈ heteroaryl comprising 1-4 nitrogen atoms, 1-3 oxygen atoms and/or 1-2 sulfur atoms, wherein any of the aforementioned groups is optionally further substituted with the proviso that the aforementioned total sum of the elements recited for R^{a} is not exceeded. It may also be advantageous that (L)_{nR}³ represents (CH₂)₁₋₄—O—R^{a}, O—R^{a}, C(O)R^{a}, CO₂R^{a}, or O—C(O)R^{a}, wherein R^{a} represents an optionally substituted phenyl. It may be especially advantageous that (L)ₙR¹ represents CO₂R^{a}, C(O)R^{a} or R^{a}, wherein R^{a} represents optionally substituted phenyl, in particular wherein the optionally substituted phenyl comprises 6 to 30, more specifically 6 to 16, and in particular 6 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms. In some of these embodiments, R^{a} is further substituted with one or more moieties selected from the group consisting of: —C₁₋₄ alkyl, —O—C₁₋₄ alkyl, —S—C₁₋₄ alkyl, —NH—C₁₋₄ alkyl, —N(C₁₋₄ alkyl)₂, —C(O)C₁₋₄ alkyl, —CO₂C₁₋₄ alkyl, —O₂C—C₁₋₄ alkyl, —C(O)NH—C₁₋₄ alkyl, —C(O)N(C₁₋₄ alkyl)₂, —NH—C(O)C₁₋₄ alkyl, —N(C₁₋₄ alkyl)-C(O)C₁₋₄ alkyl, —SO₃H, —NO₂, —NH₂, —OH, —SH, —COOH, —C(O)H, halogen, in particular Cl, Br or F; and —CF₃.

In some embodiments, the present disclosure provides compounds that are derivatives of genipin. As used herein, “genipin” also refers to genipin extract, partially purified genipin extract, in addition to the purified compound genipin as depicted above. Genipin is derived from an iridoid glycoside called geniposide, which is present in nearly 40 plant species. Additionally, genipin is a precursor to gardenia blue that undergoes a transformative reaction upon exposure to amine groups to form a blue colorant (see, for example, Touyama et al., Chem. Pharm. Bull. 42:668-673, 1994; and Touyama et al., Chem. Pharm. Bull. 42(8):1571-1578, 1994; both of which are incorporated by reference herein in their entireties). When placed in contact with skin, genipin can react with the amines in skin to generate the polymer-based and/or oligomer-based blue color in situ.

In some embodiments, the present disclosure provides compounds that are derivatives of de-glycosylated oleuropein, also known as oleuropein aglycone, or compound methyl (4s,5e,6r)-4-[2-[2-(3,4-dihydroxyphenyl)ethoxy]-2-oxoethyl]-5-ethylidene-6-hydroxy-4h-pyran-3-carboxylate (CAS RN. 31773-95-2) having the following chemical structure:

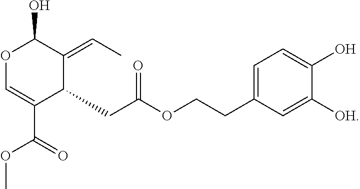 [SMILES: C/C=C1/[C@H](O)OC=C(C(=O)OC)[C@H]1CC(=O)OCCc1ccc(O)c(O)c1]

Generally, oleuropein is a glycosylated seco-iridoid, a type of phenolic bitter compound found in green olive skin, flesh, seeds, and leaves, and argan oil. This compound can be de-glycosylated using appropriate chemical reagents to obtain the oleuropein aglycone shown above.

### Compounds of Formula (IV)

In some embodiments, the compound of formula (II) is a compound of Formula (IV):

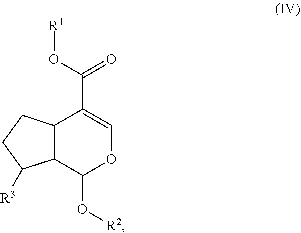

or a pharmaceutically acceptable salt thereof, wherein: R¹ is selected from H and a carboxylic acid-protecting group; R² is as defined for formula (II) above; and R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2}; or wherein R³ and OR², together with the carbon atoms to which they are attached form a tetrahydrofuran ring, which is optionally substituted with OR^{a1} or oxo; each R^{a1} is independently selected from H, C₁₋₆ alkyl, C₁₋₄haloalkyl, C₂₋₆ alkenyl, C₂₋₆ alkynyl, and a hydroxyl-protecting group; each R^{a2} is independently selected from H, C₁₋₆ alkyl, C₁₋₄ haloalkyl, C₂₋₆ alkenyl, C₂₋₆ alkynyl, and a carboxyl-protecting group; each R^{c1} and R^{d1} is independently selected from H, C₁₋₆ alkyl, C₁₋₄ haloalkyl, C₂₋₆ alkenyl, C₂₋₆ alkynyl, and an amino-protecting group; and each R^{b1} is independently selected from H, C₁₋₆ alkyl, C₁₋₄ haloalkyl, C₂₋₆ alkenyl, and C₂₋₆ alkynyl.

In some embodiments:
R¹ is selected from H and a carboxylic acid-protecting group;
R² is selected from H, C₁₋₆ alkyl, and a hydroxyl-protecting group; or
R³ and OR², together with the carbon atoms to which they are attached form a tetrahydrofuran ring, which is optionally substituted with OR^{a1} or oxo;
R³ is selected from C₁₋₆ alkyl, C₂₋₆ alkenylene, C₂₋₆ alkynylene, C(O)R^{b1}, C(O)NR^{c1}R^{d1}, and C(O)OR^{a2}, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

In some embodiments, R³ and OR², together with the carbon atoms to which they are attached form a tetrahydrofuran ring, which is optionally substituted with OR^{a1} or oxo.

In some embodiments, the compound of Formula (IV) is not any one of the following compounds:

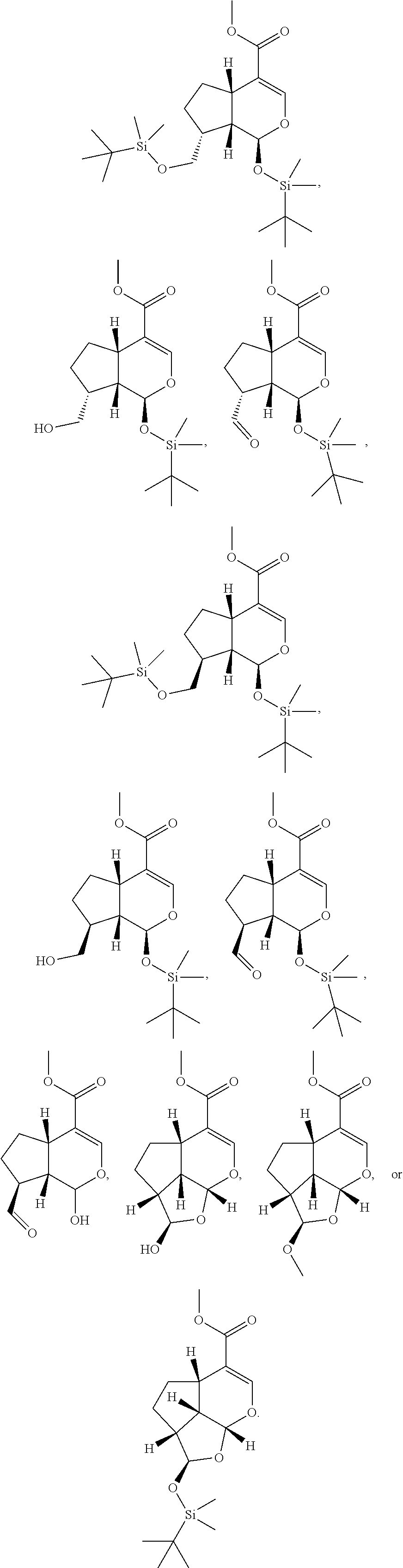

Suitable examples of carboxylic acid protecting groups include methyl ester, t-butyl ester, and benzyl ester. In some embodiments, R¹ is C₁₋₆ alkyl.

Suitable examples of hydroxyl protecting groups include methoxymethyl ether (MOM), benzyloxymethyl ether (BOM), benzyl ether, p-methoxybenzyl ether (PMB), trityl ether, silyl ether (e.g., TMS, TIPS, or TBDMS), and an acyl group. In some embodiments, R² is selected from methoxymethyl, benzyloxymethyl, benzyl, p-methoxybenzyl, tri(C₁₋₆ alkyl)silyl, and an acyl. Suitable examples of tri(C₁₋₆ alkyl)silyls include trityl, trimethylsilyl and triisopropylsilyl. Suitable examples of acyl groups include formyl, acetyl, propionyl, acrylyl, pivaloyl, or benzoyl. In some embodiments, R² is C₁₋₆ alkyl. In some embodiments, R² is acyl.

Suitable examples of amino protecting groups include 9-fluorenylmethoxycarbonyl (Fmoc), the tert-butyloxycarbonyl (Boc), benzyl, benzyl carbamate (Cbz), phthalimide, p-toluenesulfonamide (Ts), and triphenylmethylamine (Tr).

Additional examples of carboxyl protecting groups, hydroxyl protecting groups, and amino protecting groups are described, for example, in P. G. M. Wuts and T. W. Greene, Protective Groups in Organic Synthesis, 4^{th} ed., Wiley & Sons, Inc., New York (2006), which is incorporated herein by reference in its entirety.

In some embodiments, R³ is C₂₋₆ alkenylene.

In some embodiments, R³ is C₂₋₆ alkynylene.

In some embodiments, R³ is C(O)R^{b1}.

In some embodiments, R³ is C(O)NR^{c1}R^{d1}.

In some embodiments, R³ is C(O)OR^{a2}.

In some embodiments, R³ is selected from C(O)R^{b1}, C(O)NR^{c1}R^{d1}, and C(O)OR^{a2}.

In some embodiments, R³ is C₁-6 alkyl, optionally substituted with OR^{a1} or NR^{c1}R^{d1}. In some embodiments, R³ is C₁₋₆ alkyl. In some embodiments, R³ is C₁₋₆ alkyl, optionally substituted with a substituent selected from C(O)R^{b1}, C(O)NR^{c1}R^{d1}, and C(O)OR^{a2}.

In some embodiments, R³ is C₁₋₆ alkyl substituted with OR^{a1}.

In some embodiments, R³ is C₁₋₆ alkyl substituted with NR^{c1}R^{d1}.

In some embodiments, R^{a1} is selected from H and C₁₋₆ alkyl. In some embodiments, R^{a1} is H. In some embodiments, R^{a1} is C₁₋₆ alkyl.

In some embodiments, R^{a1} is a hydroxyl-protecting group. In some embodiments, R^{a1} is selected from methoxymethyl, benzyloxymethyl, benzyl, p-methoxybenzyl, tri(C₁₋₆ alkyl)silyl, and an acyl. Suitable examples of silyls and acyls are described above. In some embodiments, R^{a1} is selected from H, acyl, and tri(C₁₋₆ alkyl)silyl. In some embodiments, R^{a1} is selected from H and acyl.

In some embodiments, R^{a2} is selected from H and C₁₋₆ alkyl. In some embodiments, R^{a2} is H. In some embodiments, R^{a2} is C₁₋₆ alkyl. In some embodiments, R^{a2} is a carboxyl-protecting group.

In some embodiments, R^{c1} and R^{d1} are each independently selected from H, C₁₋₆ alkyl, an amino-protecting group.

In some embodiments, R^{c1} is H or C₁₋₆ alkyl, and R^{d1} is an amino-protecting group. In some aspects of these embodiments, R^{d1} is selected from 9-fluorenylmethoxycarbonyl (Fmoc), the tert-butyloxycarbonyl (Boc), benzyl, benzyl carbamate (Cbz), phthalimide, p-toluenesulfonamide (Ts), and triphenylmethylamine (Tr).

In some embodiments, R^{b1} is selected from H and C₁₋₆ alkyl. In some embodiments, R^{b1} is H. In some embodiments, R^{b1} is C₁₋₆ alkyl.

In some embodiments:
R¹ is selected from H and C₁₋₆ alkyl;
R² is selected from H and a hydroxyl-protecting group;
R³ is selected from C₁₋₆ alkyl, C₂₋₆ alkenylene, C₂₋₆ alkynylene, C(O)OR^{a2}, and C(O)R^{b1}, wherein said C₁₋₆ alkyl is optionally substituted with OR^{a1} or NR^{c1}R^{d1};
R^{a1} is selected from H and a hydroxyl-protecting group;
R^{a2} is selected from H, C₁₋₆ alkyl, and a carboxyl-protecting group;
R^{b1} is selected from H and C₁₋₆ alkyl;
R^{c1} is H or C₁₋₆ alkyl; and
R^{d1} is an amino-protecting group.

In some embodiments:
R¹ is selected from H and C₁₋₆ alkyl;
R² is selected from H, methoxymethyl, benzyloxymethyl, benzyl, p-methoxybenzyl, tri(C₁₋₆ alkyl)silyl, and an acyl;
R³ is selected from C₁₋₆ alkyl, C(O)R^{b1}, C(O)OR^{a2}, C₁₋₆ alkylene-OR^{a1}, and C₁₋₆ alkylene-NR^{c1}R^{d1};
R^{a1} is selected from H, methoxymethyl, benzyloxymethyl, benzyl, p-methoxybenzyl, tri(C₁₋₆ alkyl)silyl, and an acyl;
R^{a2} is selected from H and C₁₋₆ alkyl;
R^{b1} is selected from H and C₁₋₆ alkyl;
R^{c1} is H or C₁₋₆ alkyl; and
R^{d1} is selected from 9-fluorenylmethoxycarbonyl (Fmoc), the tert-butyloxycarbonyl (Boc), benzyl, benzyl carbamate (Cbz), phthalimide, p-toluenesulfonamide (Ts), and triphenylmethylamine (Tr).

In some embodiments, R³ is selected from C₁₋₆ alkyl, C₂₋₆ alkenylene, C₂₋₆ alkynylene, C(O)NR^{c1}R^{d1}, and C(O)OR^{a2}, wherein each of said C₁₋₆ alkyl, C₂₋₆ alkenylene, and C₂₋₆ alkynylene is optionally substituted with NR^{c1}R^{d1}, C(O)R^{b1}, C(O)NR^{c1}R^{d1}, and C(O)OR^{a2}.

In some embodiments, R² is a C₁₋₆ acyl.

In some embodiments:
R² is selected from H and C₁₋₆ acyl; and
R³ is selected from C₁₋₆ alkyl, C₂₋₆ alkenylene, C₂₋₆ alkynylene, C(O)NR^{c1}R^{d1}, and C(O)OR^{a2}, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2}.

In some embodiments, R³ and OR², together with the carbon atoms to which they are attached form a tetrahydrofuran ring, which is optionally substituted with OR^{a3} or oxo; wherein R^{a3} is an acyl.

In some embodiments, R³ and OR², together with the carbon atoms to which they are attached form a tetrahydrofuran ring, which is optionally substituted with oxo.

In some embodiments, the compound of Formula (IV) is selected from any one of the following compounds, or a pharmaceutically acceptable salt thereof:

| Cmpd. # | Structure |
|---|---|
| 1 | 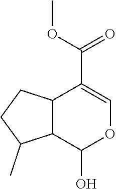 |
| 2 | 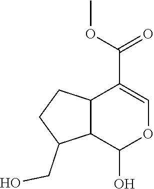 [SMILES: COC(=O)C1=COC(O)C2C(CO)CCC12] |
| 2a | 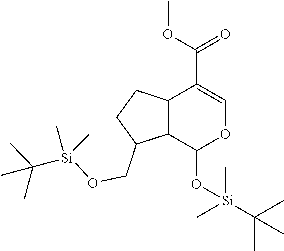 [SMILES: COC(=O)C1=COC(O[Si](C)(C)C(C)(C)C)C2C(CO[Si](C)(C)C(C)(C)C)CCC12] |
| 2b | 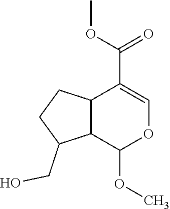 [SMILES: COC(=O)C1=COC(OC)C2C(CO)CCC12] |
| 6 | 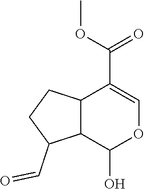 [SMILES: COC(=O)C1=COC(O)C2C(C=O)CCC12] |
| 7 | 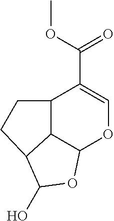 [SMILES: COC(=O)C1=COC2OC(O)C3CCC1C23] |
| 6a | 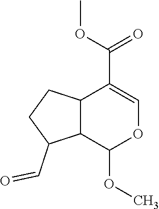 [SMILES: COC(=O)C1=COC(OC)C2C(C=O)CCC12] |
| 6b | 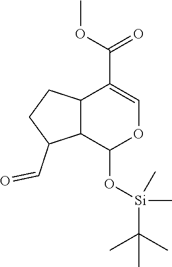 [SMILES: COC(=O)C1=COC(O[Si](C)(C)C(C)(C)C)C2C(C=O)CCC12] |
| 8 | 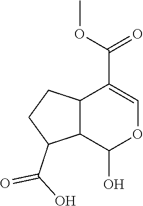 [SMILES: COC(=O)C1=COC(O)C2C(C(=O)O)CCC12] |
| 8a | 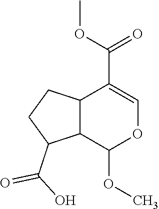 [SMILES: COC(=O)C1=COC(OC)C2C(C(=O)O)CCC12] |
| 9 | 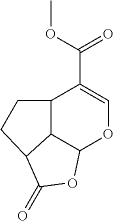 [SMILES: COC(=O)C1=COC2OC(=O)C3CCC1C23] |

In some embodiments, the compound of Formula (IV) is selected from any one of the following compounds, or a pharmaceutically acceptable salt thereof:

| Cmpd. # | Structure |
|---|---|
| | 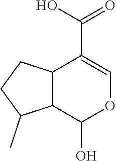 |
| | 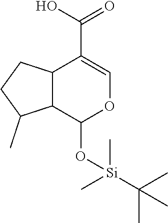 [SMILES: CC1CCC2C(C(=O)O)=COC(O[Si](C)(C)C(C)(C)C)C12] |
| | 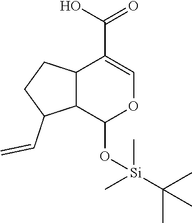 [SMILES: C=CC1CCC2C(C(=O)O)=COC(O[Si](C)(C)C(C)(C)C)C12] |
| 4 | 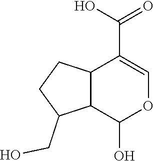 [SMILES: O=C(O)C1=COC(O)C2C(CO)CCC12] |
| | 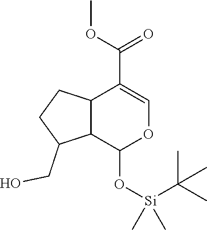 [SMILES: COC(=O)C1=COC(O[Si](C)(C)C(C)(C)C)C2C(CO)CCC12] |
| | 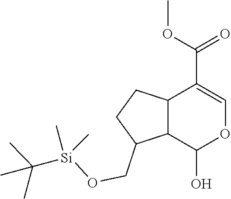 [SMILES: COC(=O)C1=COC(O)C2C(CO[Si](C)(C)C(C)(C)C)CCC12] |
| | 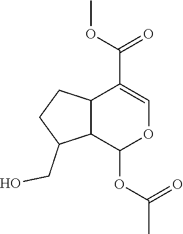 [SMILES: COC(=O)C1=COC(OC(C)=O)C2C(CO)CCC12] |
| | 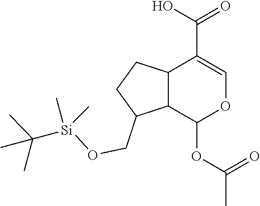 [SMILES: CC(=O)OC1OC=C(C(=O)O)C2CCC(CO[Si](C)(C)C(C)(C)C)C12] |
| | 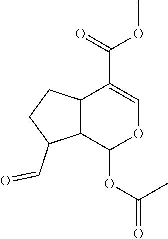 [SMILES: COC(=O)C1=COC(OC(C)=O)C2C(C=O)CCC12] |
| | 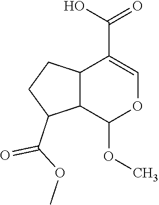 [SMILES: COC(=O)C1CCC2C(C(=O)O)=COC(OC)C12] |
| | 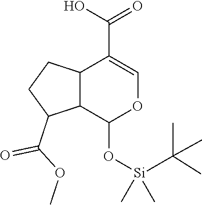 [SMILES: COC(=O)C1CCC2C(C(=O)O)=COC(O[Si](C)(C)C(C)(C)C)C12] |
| | 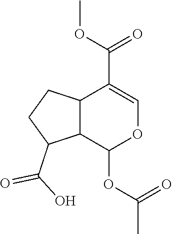 [SMILES: COC(=O)C1=COC(OC(C)=O)C2C(C(=O)O)CCC12] |
| | 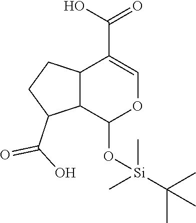 [SMILES: CC(C)(C)[Si](C)(C)OC1OC=C(C(=O)O)C2CCC(C(=O)O)C12] |
| 5 | 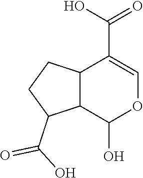 [SMILES: O=C(O)C1=COC(O)C2C(C(=O)O)CCC12] |
| | 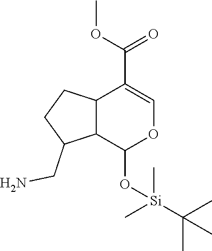 [SMILES: COC(=O)C1=COC(O[Si](C)(C)C(C)(C)C)C2C(CN)CCC12] |
| | 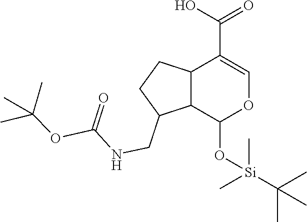 [SMILES: CC(C)(C)OC(=O)NCC1CCC2C(C(=O)O)=COC(O[Si](C)(C)C(C)(C)C)C12] |

In some embodiments, the present disclosure provides a compound of Formula (V) or Formula (VI):

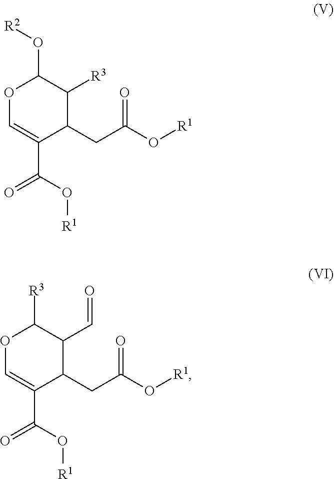

or a salt (e.g., a pharmaceutically acceptable salt) thereof, wherein R¹, R², and R³ are as described herein for formula (IV).

It should be understood that in those instances of the above disclosure where merely reference to the compound as such is made, the present disclosure also provides the same compounds comprised in a topical composition, in particular in combination with an excipient suitable for topical administration.

### Methods of Using a Compound of Formula (IV)

Generally, any of the compounds of Formula (IV) may also be considered as a synthetic intermediate useful for making any of the compounds of Formula (I), Formula (II), Formula (VII), Formula (VIII), Formula (IX), or Formula (X), or their salts, as described further herein. For example, any of the functional groups within R¹, R², and R³ of Formula (IV) may be considered as reactive chemical groups, or protected forms thereof. A skilled synthetic chemist would be able to select appropriate protection/deprotection protocols and synthetic routes to successfully use any of the compounds of Formula (IV) for making compounds of Formula (VII) or Formula (VIII). In some embodiments, the compound of Formula (IV) has Formula (XI). In some embodiments, the compound of Formula (IV) has Formula (XII).

In some embodiments, the present disclosure provides a method of making a compound of Formula (VII) or Formula (IX):

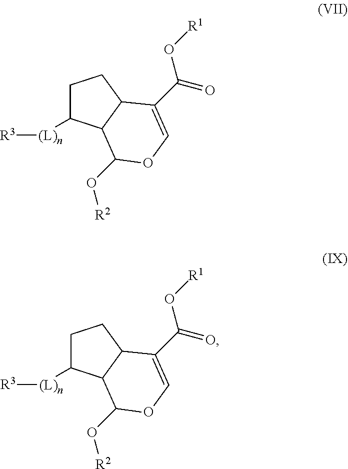

or a pharmaceutically acceptable salt thereof, wherein R¹, R², R³, L, and n are as described herein for Formula (VII) or Formula (IX), the method comprising:
reacting a compound of Formula (XI):

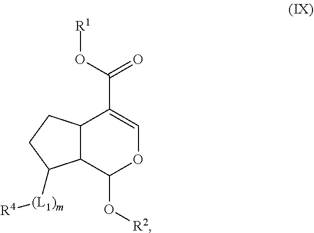

wherein:
R¹ and R² are as described herein for Formula (IV);
R⁴ is a first reactive group; and
each L¹ is independently selected from any of L, and m is selected from 0 and any of n;
with a compound of Formula R³-(L²)ₚ-R⁵, wherein:
R⁵ is a second reactive group;
each L² is independently selected from any of L, and p is selected from 0 and any of n,
to obtain the compound of Formula (VII) or Formula (IX).

In some embodiments, m is 0. In some embodiments, m is selected from 1, 2, 3, 4, 5, or 6. In some embodiments, m is 1. In some embodiments, m is 2.

In some embodiments, p is 0. In some embodiments, p is selected from 1, 2, 3, 4, 5, or 6. In some embodiments, p is 1. In some embodiments, p is 2.

In some embodiments, the sum of m and p is n. In some embodiments, (L¹)ₘ is the same as (L)ₙ. In some embodiments, (L²)ₚ is the same as (L)ₙ.

In some embodiments, the first reactive group is selected from any of R³ as described herein Formula (IV).

In some embodiments, the first reactive group is selected from C(O)H, C(O)OH, OH, NH₂, SH, C(O)Hal, CH₂Hal, Michael acceptor, and an activated ester. In some embodiments, the first reactive group is C(O)H. In some embodiments, the first reactive group is selected from C(O)OH, C(O)Hal, and an activated ester. In some embodiments, the first reactive group is selected from OH and NH₂. Suitable examples of Michael acceptors include an enone and a maleimide.

In some embodiments, the first reactive group is a click-reactive group. Suitable examples of those groups include azide (—N₃), an alkyne, a nitrone, an isocyanide, a cyclopropene and a tetrazine. In some aspects, the alkyne is an aliphatic alkyne or a cyclooctyne. In some aspects, the cyclooctyne is dibenzocyclooctyne (DBCO), difluorobenzocyclooctyne (DIFBO), biarylazacyclooctynone (BARAC), dibenzocyclooctyne (DIBO), difluorinated cyclooctyne (DIFO), monofluorinated cyclooctyne (MOFO), dimethoxyazacyclooctyne (DIMAC) or aryl-less octyne (ALO).

In some embodiments, the second reactive group is selected from C(O)H, C(O)OH, OH, NH₂, SH C(O)Hal, CH₂Hal, a Michael acceptor, and an activated ester. In some embodiments, the second reactive group is C(O)H. In some embodiments, the second reactive group is selected from C(O)OH, C(O)Hal, and an activated ester. In some embodiments, the second reactive group is selected from OH and NH₂. In some embodiments, the second reactive group is a Michael acceptor.

In some embodiments, the second reactive group is a click-reactive group. Suitable examples of those second reactive groups include azide (—N₃), an alkyne, a nitrone, an isocyanide, a cyclopropene and a tetrazine. In some aspects, the alkyne is an aliphatic alkyne or a cyclooctyne. In some aspects, the cyclooctyne is dibenzocyclooctyne (DBCO), difluorobenzocyclooctyne (DIFBO), biarylazacyclooctynone (BARAC), dibenzocyclooctyne (DIBO), difluorinated cyclooctyne (DIFO), monofluorinated cyclooctyne (MOFO), dimethoxyazacyclooctyne (DIMAC) or aryl-less octyne (ALO).

In some embodiments, the Hal is selected from Cl, Br, F, and I. In some embodiments, the activated ester is selected from p-nitrophenol ester and N-hydroxysuccinimide (NHS) ester.

In some embodiments, the first reactive group is complementary of the second reactive group. As used herein, the term “complementary” refers to the ability of the first group to react with the second group to form a stable covalent bond.

In some embodiments, the first reactive group is selected from C(O)OH, C(O)Hal, and an activated ester; and the second reactive group is selected from OH and NH₂.

In some embodiments, the first reactive group is selected from OH and NH₂, and the second reactive group is selected from C(O)OH, C(O)Hal, and an activated ester.

In some embodiments, the first reactive group is CH₂Hal and the second reactive group is selected from OH, SH, and NH₂. In some embodiments, the second reactive group is CH₂Hal and the first reactive group is selected from OH, SH, and NH₂.

In some embodiments, the first reactive group is an azide, and the second reactive group is an alkyne. In some embodiments, the second reactive group is an azide, and the first reactive group is an alkyne.

In some embodiments, the present disclosure provides a method of making a compound of Formula (VIII) or Formula (X):

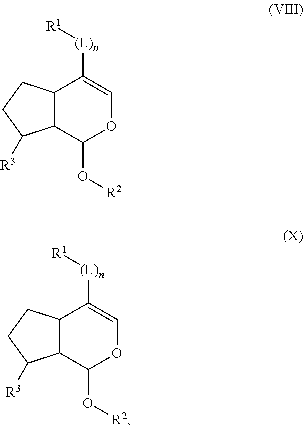

or a pharmaceutically acceptable salt thereof, wherein R¹, R², R³, L, and n are as described herein for Formula (VIII) or Formula (X), the method comprising:
reacting a compound of Formula (XII)

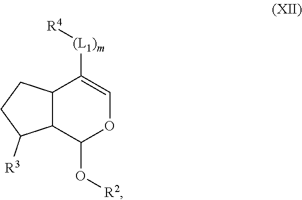

wherein:
R³ and R² are as described herein for Formula (IV);
R⁴ is a first reactive group; and
each L¹ is independently selected from any of L, and m is selected from 0 and any of n;
with a compound of Formula R¹-(L²)ₚ-R⁵, wherein:
R⁵ is a second reactive group;
each L² is independently selected from any of L, and p is selected from 0 and any of n,
to obtain the compound of Formula (VIII) or Formula (X).

In some aspects of these embodiments, each L¹, L², m, p, R⁴, and R⁵ are as described hereinabove for the method of making Formula (VII) or Formula (X).

In some embodiments, the compound of Formula (V) may be used for making compound of Formula (XIII) using the methods and procedures similar to those where the compound of Formula (XI) is used for making compounds of Formula (VII) or Formula (IX). In some embodiments, the compound of Formula (VI) may be used for making compound of Formula (XIV) using the methods and procedures similar to those where the compound of Formula (XII) is used for making compounds of Formula (VIII) or Formula (X).

The aforementioned methods of synthesis may also serve as templates for synthesizing the compounds of formula (I).

In some embodiments, the present disclosure provides a topical composition comprising a compound of Formula (IV), (V), or (VI), or a pharmaceutically acceptable salt thereof, and an excipient suitable for topical administration. For example, any of the compound of Formula (IV), (V), or (VI), or a pharmaceutically acceptable salt thereof, may be formulated in a sunscreen composition as described herein for Formulae (IX)—(XIV). These compounds can also be used for protecting skin of a subject from UV light, comprising applying a topical composition with said compound to the skin of the subject.

### Dye Conjugates, Compositions, and Methods

In one general aspect, the present disclosure also provides colorants, including semi-permanent colorants, that include derivatives of genipin (e.g., hydrogenated genipin). Also provided herein are compositions including such colorants that can be applied to the skin, for examples, as a semi-permanent tattoo. Such compositions can be formulated to be safe for the skin contact and sustain stability or skin transferability for the duration of the product shelf life. Also provided herein are layered adhesive articles and methods for application of the compositions described herein. In some embodiments, the compositions described herein can be suitable for application to skin using a pen-like applicator. Without being bound by any particular theory, the colorant compounds of this disclosure penetrate one or more layers of the skin and are unable to be removed from the skin without physical disruption or natural desquamation of the skin. The compounds may penetrate the stratum corneum and react with other molecules, such as collagen, keratin, or other proteins of the skin. As a result, the colorant molecules are immobilized in the stratum corneum. Generally, when the colorant compound of this disclosure penetrates the stratum corneum, the colorant residence time is determined by the natural skin desquamation process. In some embodiments, the applied compounds cannot be washed off, for example, by water, soap, and/or isopropanol.

### Compounds of Formula (VII)

In some embodiments, the present disclosure provides a compound of Formula (VII):

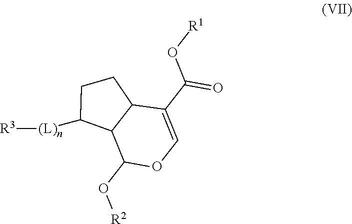

or a pharmaceutically acceptable salt thereof, wherein:
R¹ is selected from H and C₁₋₆ alkyl;
R² is selected from H and an acyl; or
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy; and
each R^{N} is independently selected from H and C₁₋₃ alkyl; and
R³ is a dye moiety.

In some embodiments, R¹ is H.

In some embodiments, R¹ is C₁₋₆ alkyl.

In some embodiments, R² is H. In some embodiments, R² is acyl. In some embodiments, R² is selected from H, formyl, acetyl, propionyl, acrylyl, pivaloyl, and benzoyl.

In some embodiments, at least one L is —C₁₋₃ alkylene-. In some embodiments, at least one L is methylene. In some embodiments, at least one L is ethylene.

In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, at least one L is C(═O). In some embodiments, at least one L is NH. In some embodiments, at least one L is 0. In some embodiments, at least one L is (—C₁₋₃ alkylene-O—)ₓ. In some embodiments, at least one L is (—O—C₁₋₃ alkylene-)ₓ. In some embodiments, at least one L is (—CH₂CH₂—O—)ₓ. In some embodiments, at least one L is (—O—CH₂CH₂—)ₓ. In some embodiments, x is an integer from 1 to 5. In some embodiments, x is 1, 2, or 3. In some embodiments, (L)ₙ comprises at least one moiety —(C═O)NH—. In some embodiments, (L)ₙ comprises at least one moiety —(C═O)O—. In some embodiments, (L)ₙ comprises at least one moiety —CH₂O—.

In some embodiments, the R³ in Formula (VII) is a dye having medium to high molecular weight. Examples of such dyes include azo, diazo, anthraquinone, diphenylamine, nitroarylamine, coumarin, di- or triarylmethane, naphthostyryl, quinophthalone, formazan, quinoline, indigo, indoline, or benzodifuranone-based dye.

These dyes generally represent a full range of rainbow colors of various intensity, including red, orange, yellow, green, blue, indigo, violet, or any combination and/or a shade thereof. A dye having any color within the wavelength from about 390 nm (violet) to about 800 nm (red) is encompassed. Generally, the dye within R³ contains, or can be chemically converted to contain, a reactive functional group which is used to attach the dye to the remainder of the molecule of Formula (VII). Suitable examples of such groups include a hydroxyl group (—OH), an amino group (—NH₂), a carboxylic acid group (—C(═O)OH), a sulfonic acid group (—S(═O)₂OH), and a phosphonate group (—P(═O)(OH)₂). For example, a dye having a carboxylic acid group can be attached to the linker moiety of Formula (Ia-b) by reacting with an amino group of the linker moiety to form an amide bond. In a similar manner, a dye having a reactive hydroxyl group can be attached to the linker moiety by reacting with a carboxylic acid group of the linker moiety to form an ester. A skilled organic chemist would be able to select the appropriate reagents and functional groups for attaching a dye to the linker moiety of Formula (VII), and would be able to implement the appropriate synthetic protocols. A dye within formula (VII) can be any dye generally known in the tattoo industry for being able to color a human skin. In one example, the dye may be any of the dye or colorants approved by FDA or by EMA for human use, such as a food coloring. Suitable examples of such dyes include FD&C Blue No. 1, FD&C Blue No. 2, FD&C Green No. 3, Orange B, FD&C Red No. 40, FD&C Yellow No. 5, FD&C Yellow No. 6, and similar colorants.

In some embodiments, R³ is selected from any one of the following moieties:

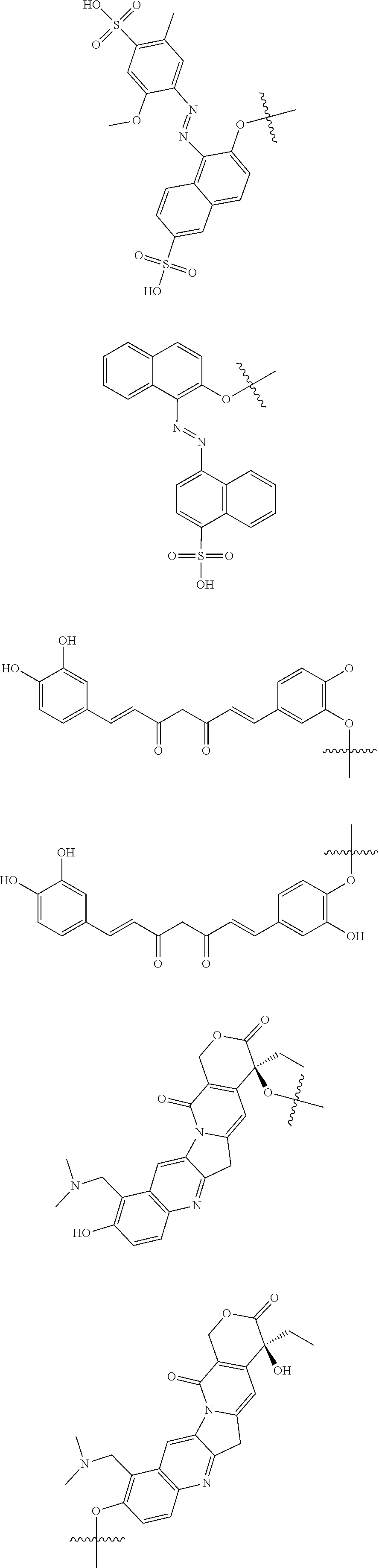

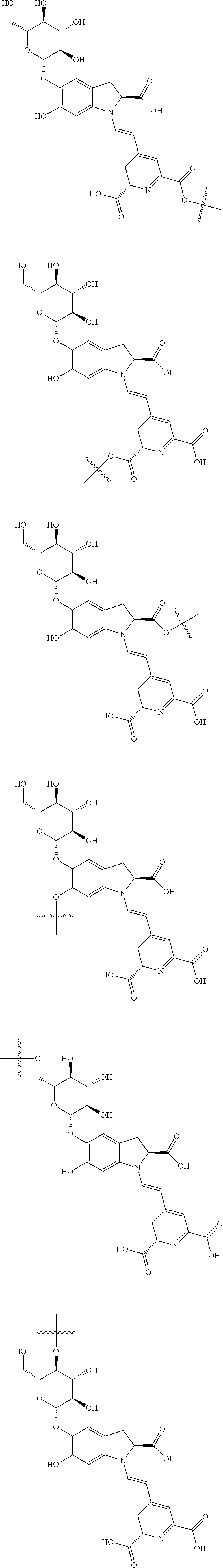

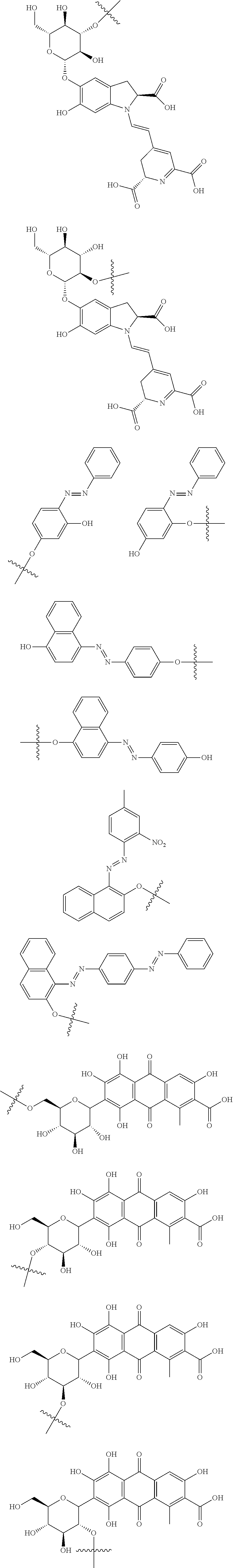

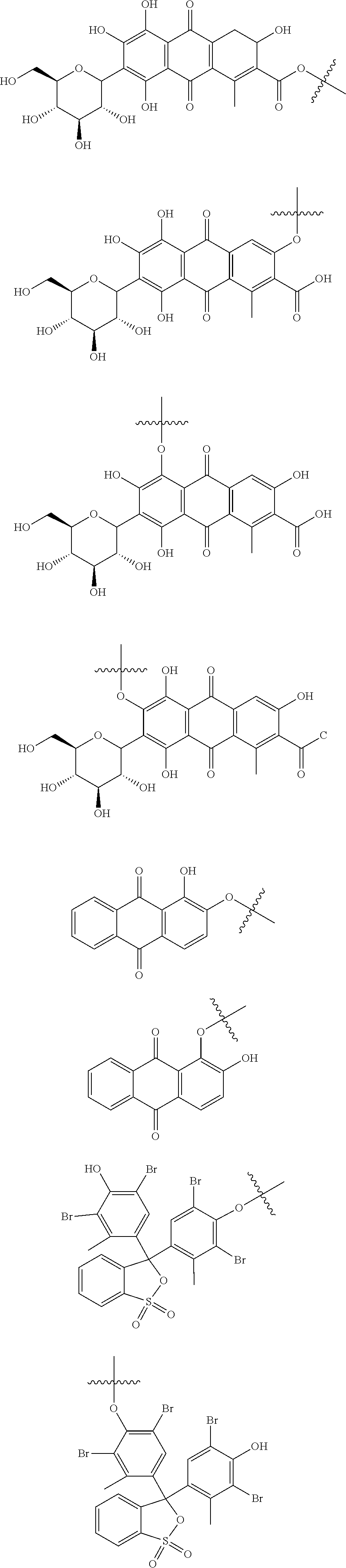

In some embodiments, the compound of Formula (VII) is selected from any one of the following formulae:

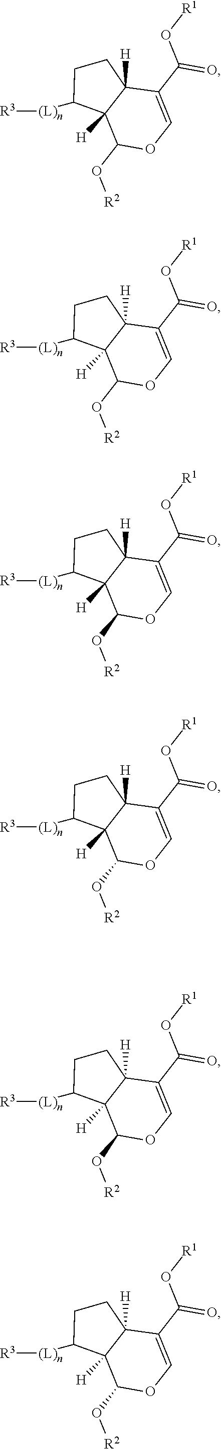

or a pharmaceutically acceptable salt thereof.

In some embodiments, the compound of Formula (VII) is selected from any one of the following compounds:

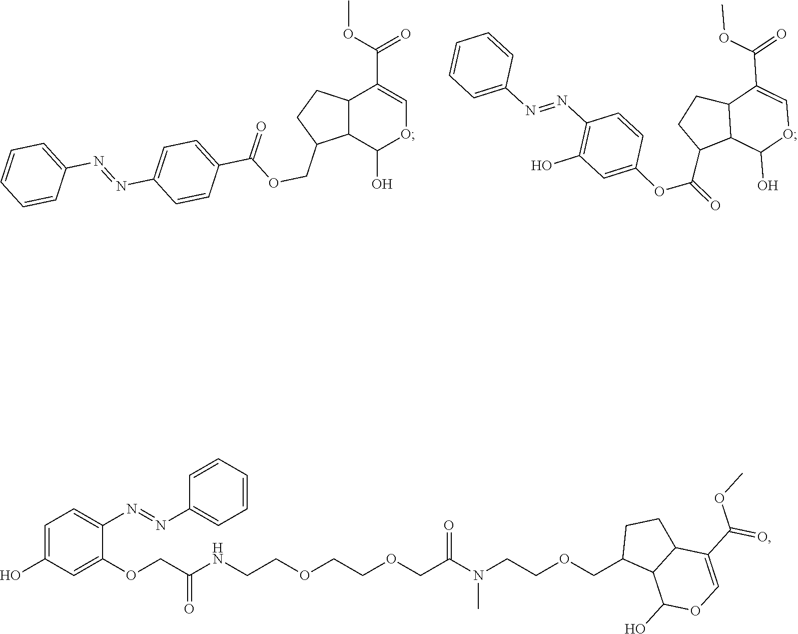 [SMILES: COC(=O)C1=COC(O)C2C(COC(=O)c3ccc(/N=N/c4ccccc4)cc3)CCC12 ; COC(=O)C1=COC(O)C2C(C(=O)Oc3ccc(/N=N/c4ccccc4)c(O)c3)CCC12 ; COC(=O)C1=COC(O)C2C(COCCN(C)C(=O)COCCOCCNC(=O)COc3cc(O)ccc3/N=N/c3ccccc3)CCC12]

or a pharmaceutically acceptable salt thereof.

### Compounds of Formula (VIII)

In some embodiments, the present disclosure provides a compound of Formula (VIII):

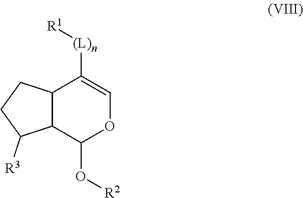

or a pharmaceutically acceptable salt thereof, wherein:
R¹ is a dye moiety;
R² is selected from H, C₁₋₆ alkyl, and an acyl; or
R³ and OR², together with the carbon atoms to which they are attached form a tetrahydrofuran ring, which is optionally substituted with OR^{a1} or oxo;
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is selected from C₁₋₆ alkyl, C₂₋₆ alkenylene, C₂₋₆ alkynylene, C(O)R^{b1}, C(O)NR^{c1}R^{d1}, and C(O)OR^{a2}, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
each R^{a1} is independently selected from H, C₁₋₆ alkyl, C₁₋₄ haloalkyl, C₂₋₆ alkenyl, and C₂₋₆ alkynyl;
each R^{a2} is independently selected from H, C₁₋₆ alkyl, C₁₋₄ haloalkyl, C₂₋₆ alkenyl, and C₂₋₆ alkynyl;
each R^{c1} and R^{d1} is independently selected from H, C₁₋₆ alkyl, C₁₋₄haloalkyl, C₂₋₆ alkenyl, and C₂₋₆ alkynyl; and
each R^{b1} is independently selected from H, C₁₋₆ alkyl, C₁₋₄ haloalkyl, C₂₋₆ alkenyl, and C₂₋₆ alkynyl.

In some embodiments, R³ and OR², together with the carbon atoms to which they are attached form a tetrahydrofuran ring, which is optionally substituted with OR^{a1} or oxo.

In some embodiments, R² is H. In some embodiments, R² is acyl. In some embodiments, R² is selected from H, formyl, acetyl, propionyl, acrylyl, pivaloyl, and benzoyl. In some embodiments, R² is C₁₋₆ alkyl.

In some embodiments, at least one L is —C₁₋₃ alkylene-. In some embodiments, at least one L is methylene. In some embodiments, at least one L is ethylene.

In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, at least one L is C(═O). In some embodiments, at least one L is NH. In some embodiments, at least one L is O. In some embodiments, at least one L is (—C₁₋₃ alkylene-O—)ₓ. In some embodiments, at least one L is (—O—C₁₋₃ alkylene-)ₓ. In some embodiments, at least one L is (—CH₂CH₂—O—)ₓ. In some embodiments, at least one L is (—O—CH₂CH₂—)ₓ. In some embodiments, x is an integer from 1 to 5. In some embodiments, x is 1, 2, or 3. In some embodiments, (L)ₙ comprises at least one moiety —(C═O)NH—. In some embodiments, (L)ₙ comprises at least one moiety —(C═O)O—. In some embodiments, (L)ₙ comprises at least one moiety —CH₂O—.

In some embodiments, R³ is C₁₋₆ alkyl.

In some embodiments, R³ is C₂₋₆ alkenylene.

In some embodiments, R³ is C₂₋₆ alkynylene.

In some embodiments, R³ is C(O)R^{b1}.

In some embodiments, R³ is C(O)NR^{c1}R^{d1}.

In some embodiments, R³ is C(O)OR^{a2}.

In some embodiments, R³ is selected from C(O)R^{b1}, C(O)NR^{c1}R^{d1}, and C(O)OR^{a2}.

In some embodiments, R³ is C₁₋₆ alkyl, optionally substituted with OR^{a1} or NR^{c1}R^{d1}. In some embodiments, R³ is C₁₋₆ alkyl, optionally substituted with a substituent selected from C(O)R^{b1}, C(O)NR^{c1}R^{d1}, and C(O)OR^{a2}.

In some embodiments, R³ is C₁₋₆ alkyl substituted with OR^{a1}.

In some embodiments, R³ is C₁₋₆ alkyl substituted with NR^{c1}R^{d1}.

In some embodiments, R^{a1} is selected from H and C₁₋₆ alkyl. In some embodiments, R^{a1} is H. In some embodiments, R^{a1} is C₁₋₆ alkyl. In some embodiments, R^{a1} is selected from H and acyl.

In some embodiments, R^{a2} is selected from H and C₁₋₆ alkyl. In some embodiments, R^{a2} is H. In some embodiments, R^{a2} is C₁₋₆ alkyl.

In some embodiments, R^{c1} and R^{d1} are each independently selected from H; C₁₋₆ alkyl, and acyl.

In some embodiments, R^{b1} is selected from H and C₁₋₆ alkyl. In some embodiments, R^{b1} is H. In some embodiments, R^{b1} is C₁₋₆ alkyl.

In some embodiments, the R¹ in Formula (VIII) is a dye as described herein for R³ in Formula (VII).

In some embodiments:
R² is selected from H and acyl;
R³ is selected from C₁₋₆ alkyl, C₂₋₆ alkenylene, C₂₋₆ alkynylene, C(O)OR^{a2}, and C(O)R^{b1}, wherein said C₁₋₆ alkyl is optionally substituted with OR^{a1} or NR^{c1}R^{d1}; and
R^{a1}, R^{a2}, R^{b1}, R^{c1} and R^{d1} are each independently selected from is H or C₁₋₆ alkyl.

In some embodiments, R³ is selected from C₁₋₆ alkyl, C(O)OR^{a2}, C(O)R^{b1}, C₁₋₆ alkylene-OR^{a1}, and C₁₋₆ alkylene-NR^{c1}R^{d1}.

In some embodiments, the compound of Formula (VIII) is selected from any one of the following formulae:

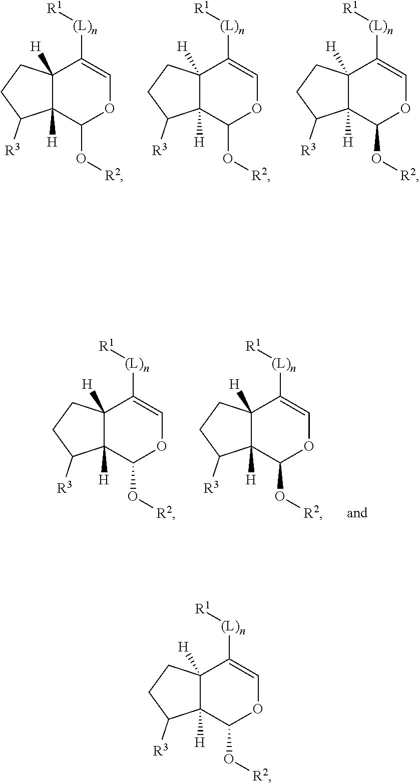

or a pharmaceutically acceptable salt thereof.

In some embodiments, the compound of Formula (VIII) is selected from any one of the following compounds:

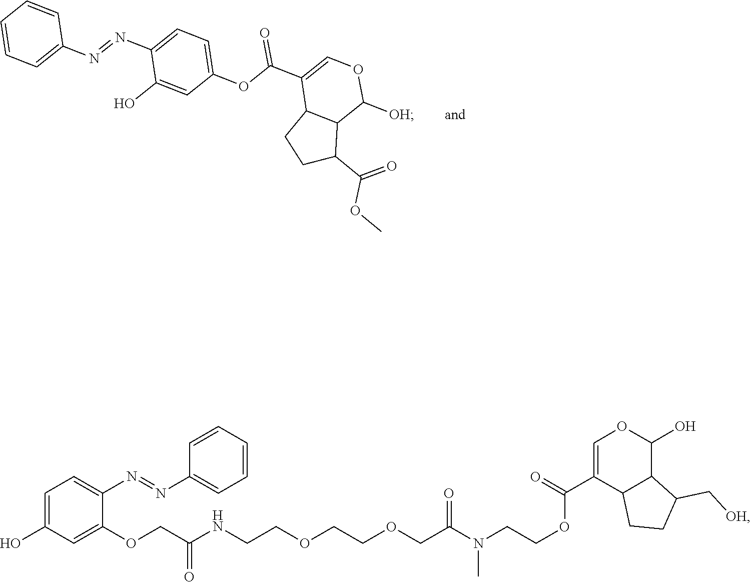 [SMILES: COC(=O)C1CCC2C(C(=O)Oc3ccc(/N=N/c4ccccc4)c(O)c3)=COC(O)C12 ; CN(CCOC(=O)C1=COC(O)C2C(CO)CCC12)C(=O)COCCOCCNC(=O)COc1cc(O)ccc1/N=N/c1ccccc1]

or a pharmaceutically acceptable salt thereof.

In some embodiments, the compound of Formula (VII) or Formula (VIII) is stable (i.e., retains its original chemical structure) after application to the subject's skin for a period of time from about 1 h to about 6 months, from about 1 day to about 3 months, from about 7 days to about 2 months, or from about 1 week to about 4 weeks. In one example, the rate of hydrolysis and/or the rate of oxidation of the compound of Formula (VII) or Formula (VIII) is very slow, allowing the dye compound in Formula (VII) or Formula (VIII) to stay in the subject's skin for the period of time described above. In some embodiments, aqueous solubility of the compound of Formula (VII) or Formula (VIII) is from about 1 g/L to about 100 g/L, from about 5 g/L to about 50 g/L, or from about 10 g/L to about 100 g/L.

### Compound of Formula (VIIa) and (VIIIa)

In some embodiments, the present disclosure provides a compound of Formula (VIIa):

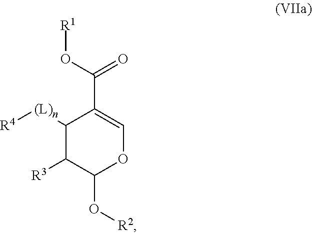

or a pharmaceutically acceptable salt thereof, wherein R¹, R², R³, L, and n are as described herein for Formula (XIII) and R⁴ is a dye moiety as described herein for R³ in Formula (VII).

In some embodiments, the present disclosure provides a compound of Formula (VIIIa):

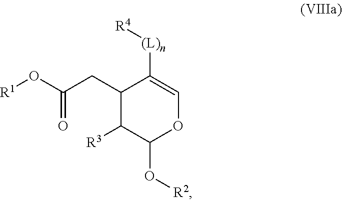

or a pharmaceutically acceptable salt thereof, wherein R¹, R², R³, L, and n are as described herein for Formula (XIV) and R⁴ is a dye moiety as described herein for R¹ in Formula (VIII).

### Methods of Using Compounds of Formula (VII), (VIIa), (VIII), and (VIIIa)

The compounds of Formula (VII)—(VIIIa), or salts thereof, are generally useful as ink compositions, and in particular as tattoo inks. As such the present disclosure provides a tattoo ink composition, comprising a compound of Formula (VII)—(VIIIa), or a pharmaceutically acceptable salt thereof, and a suitable (e.g., acceptable) carrier. Suitable examples of the acceptable carriers are those generally known as useful in tattooing. These compositions can be dry solid compositions (such as powders or dusts), which a tattooer can mix with a liquid carrier to form a liquid composition useful for application to a subject (e.g., can be applied to human skin). The compositions within the present claims can also be pre-dispersed ink compositions, comprising the compound of this disclosure and the liquid carrier. These compositions can be solutions (e.g., free of any undissolved solid particles), dispersions (e.g., containing a liquid phase and a solid precipitant phase), or emulsions.

Generally, the compositions contain water and/or organic solvents as suitable carriers and excipients. In one example, the liquid composition can be sterile and/or prepared from a sterile aqueous solution for infusion. The composition may contain from about 0.1% to about 99% w/w of the compound of Formula (VII)—(VIIIa), or a salt thereof. The composition may contain from about 0.1% to about 99% w/w of the suitable excipient (e.g., any one of the inactive ingredients described herein). Non-limiting examples of solvents useful in the compositions of this disclosure include water, methanol, ethanol, isopropanol, and diethylene glycol monoethyl ether. In some embodiments, the solvent is a volatile solvent, e.g., the solvent can evaporate or vaporize at room temperature. Non-limiting examples of volatile solvents include water, methanol, ethanol, and isopropanol. In some embodiments, the solvent is selected to have a low toxicity profile when used as described herein. In some embodiments, the solvent can include a combination of water and ethanol. The composition may also include a solubilizer, such as a polyethylene glycol, an alkyl glycol, an alkylene glycol ether, or a combination thereof. The composition may also include a surface-active agents, such as an alkylbenzene sulfonate, an alkyl sulfate, an alkyl ether sulfate, a soap, an ethoxylate, an alkyl alcohol, a lignosulfonate, or a triglyceride. The composition may also include a solid matrix. Suitable examples of a matrix component include a sugar, a sugar alcohol (e.g., sorbitol, mannitol, xylitol, isomalt, hydrogenated starch hydrolysates), a polymer, or a combination of two or more thereof. The composition may also include a skin penetration enhancer. “Skin penetration enhancer” as used herein refers to a substance that penetrates (penetrant) into skin to reversibly decrease the barrier resistance. In some embodiments, a skin penetration enhancer can also enhance the solubility of the penetrant to increase loading, which may, for example, enhance the flux of the penetrant across the skin. Non-limiting examples of a skin penetration enhancer include an alcohol, an amide, an ester, an ether alcohol, a fatty acid, a glycol, a pyrrolidone, a sulphoxide, a surfactant, and a terpene. The composition may also include a preservative, a thickening agent, a film-forming agent and/or a humectant. Non-limiting examples of thickening agents include starches, gums (e.g., natural and synthetic gums), cellulosics, and arabinogalactan. Non-limiting examples of humectants include polyhydric alcohols, for example, polyalkylene glycols (e.g., alkylene polyols and their derivatives), alpha hydroxy acids, sugars, Aloe vera gel, vegetable oil, lithium chloride, allantoin, urea, and dicyanamide. Non-limiting examples of film-forming agents include volatile silicone resins, polyvinylpyrrolidone, acrylates, acrylamides, copolymers, and isododecane resins.

The compositions can be applied to the skin of the subject using inkjet printing directly onto a skin transfer substrate. This enables production of make-to-order custom designed semi-permanent tattoos using the compounds of Formula (VII)—(VIIIa), or a salt thereof. The composition in this case is applied to the transfer substrate using printer nozzles. The composition may also by applied to the skin of the subject using a marker. In some embodiments, the ink compositions described herein can be suitable for application to the skin using a pen-like applicator. A skilled tattooer would be able to select and implement appropriate methods of application.

In some embodiments, the compositions within the instant claims may include genipin or a genipin derivative, or a salt thereof. Suitable examples of genipin derivatives useful in the ink compositions of this disclosure include those described in PCT/US2021/045767, which is incorporated herein by reference in its entirety. The composition may include, for example, form about 0.1 wt. % to about 20 wt. % of the genipin or a derivative thereof.

In some embodiments, the compositions within the instant claims may include an additional, conventional colorant, or combination thereof. The amount of the additional colorant may range from about 0.1 wt. % to about 15 wt. %. Suitable examples of those colorants include iron oxide black (Fe₃O₄), iron oxide (FeO), carbon, logwood, ochre, cinnabar (HgS), cadmium red (CdSe), iron (xIII) oxide (Fe₂O₃), naphthol-AS pigment, disazodiarylide, disazopyrazolone, cadmium seleno-sulfide, cadmium yellow (CdS, CdZnS), curcuma yellow, chrome yellow (PbCrO₄), disazodiarylide, chromium oxide (Cr₂O₃), malachite [Cu₂(CO₃)(OH)₂], a ferrocyanide, a ferricyanide, lead chromate, monoazo pigment, Cu/Al phthalocyanine, Cu phthalocyanine, azure blue, cobalt blue, Cu-phthalocyanine, manganese violet (manganese ammonium pyrophosphate), an aluminum salt, quinacridone, dioxazine/carbazole, lead white (lead carbonate), titanium dioxide (TO₂), barium sulfate (BaSO₄), zinc oxide, anthraquinone dyes and derivatives, annatto, caramel, ß-carotene, bismuth citrate, disodium EDTA copper, potassium sodium copper chlorophyllin, dihydroxyacetone, bismuth oxychloride, guaiazulene, henna, ferric ammonium ferrocyanide, ferric ferrocyanide, chromium hydroxide green, chromium oxide greens, guanine, lead acetate, pyrophillite, mica, silver, titanium dioxide, aluminum powder, bronze powder, copper powder, ultramarines, manganese violet, zinc oxide, luminescent zinc sulfide, D&C Black Nos. 2 and 3, FD&C Blue No. 1 (e.g., acid blue 9), D&C Blue No. 4, D&C Brown No. 1, FD&C Green No. 3, D&C Green Nos. 5, 6, and 8, D&C Orange Nos. 4, 5, 10 and 11, FD&C Red Nos. 4, D&C Red Nos. 6, 7, 17, 21, 22, 27, 28, 30, 32, 33, 34, 36 and 40, D&C Violet No. 2, Ext. D&C Violet No. 2, FD&C Yellow Nos. 5 and 6, D&C Yellow Nos. 7, 8, 10 and 11, and Ext. D&C Yellow No. 7. In some embodiments, the conventional colorant does not contain an amine group.

### Sunscreen Conjugates, Compositions and Methods

In one general aspect, the present disclosure provides UV-sunlight absorbing compounds that include derivatives of genipin and oleuropein. Also provided herein are compositions including such UV-sunlight absorbing compounds that can be applied to the skin, for example, as a sunscreen for protecting skin from damaging UV radiation. Such compositions can be formulated to be safe for skin contact and sustain stability or skin transferability for the duration of the product shelf life. Without being bound by any particular theory, the UV-sunlight absorbing compounds of this disclosure penetrate one or more layers of the skin and are unable to be removed from the skin without physical disruption or natural desquamation of the skin. The compound may penetrate the stratum corneum and react with other molecules, such as collagen, keratin, or other proteins of the skin. As a result, the sunscreen molecule is immobilized in the stratum corneum, providing long-term protection from sunlight.

Generally, when the sunscreen compound of this disclosure penetrates the stratum corneum, the sunscreen residence time is determined by the natural skin desquamation process. In some embodiments, the applied compound cannot be washed off, for example, by water, soap, and/or isopropanol.

### Compounds of Formula (IX):

In some embodiments, the present disclosure provides a compound of Formula (IX):

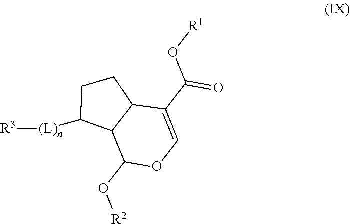

or a pharmaceutically acceptable salt thereof, wherein:
R¹ is selected from H and C₁₋₆ alkyl;
R² is selected from H and an acyl;
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is a UV-absorbing moiety.

In some embodiments, R¹ is H.

In some embodiments, R¹ is C₁₋₆ alkyl.

In some embodiments, R² is H. In some embodiments, R² is acyl. In some embodiments, R² is selected from H, formyl, acetyl, propionyl, acrylyl, pivaloyl, and benzoyl.

In some embodiments, at least one L is —C₁₋₃ alkylene-. In some embodiments, at least one L is methylene. In some embodiments, at least one L is ethylene.

In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, at least one L is C(═O). In some embodiments, at least one L is NH. In some embodiments, at least one L is O. In some embodiments, at least one L is (—C₁₋₃ alkylene-O—)ₓ. In some embodiments, at least one L is (—O—C₁₋₃ alkylene-)ₓ. In some embodiments, at least one L is (—CH₂CH₂—O—)ₓ. In some embodiments, at least one L is (—O—CH₂CH₂—)ₓ. In some embodiments, x is an integer from 1 to 5. In some embodiments, x is 1, 2, or 3. In some embodiments, (L)ₙ comprises at least one moiety —(C═O)NH—. In some embodiments, (L)ₙ comprises at least one moiety —(C═O)O—. In some embodiments, (L)ₙ comprises at least one moiety —CH₂O—.

In some embodiments, the R³ in Formula (IX) is UV-absorbing moiety such as an aryl-based or heteroaryl-based moiety having medium to high molecular weight. Examples of such moieties include phenyl, phenol, benzophenone, styrene, aminophenyl, aminophenol, indole, bendamidazole, benzotriazol, or triazine-based sunscreen moieties. These UV-absorbing molecules generally may absorb UV light of any wavelength in the UV region, such as from about 10 nm to about 400 nm, or a range thereof, in particular the range of 280 to 379 nm. Generally, the UV-absorbing moiety within R³ contains, or can be chemically converted to contain, a reactive functional group which is used to attach the UV-absorbing moiety to the remainder of the molecule of Formula (IX). Suitable examples of such groups include a hydroxyl group (—OH), an amino group (—NH₂), a carboxylic acid group (—C(═O)OH), a sulfonic acid group (—S(═O)₂OH), and a phosphonate group (—P(═O)(OH)₂). For example, a UV-absorber having a carboxylic acid group can be attached to the linker moiety of Formula (IX) by reacting with an amino group of the linker moiety to form an amide bond. In a similar manner, a UV-absorber having a reactive hydroxyl group can be attached to the linker moiety by reacting with a carboxylic acid group of the linker moiety to form an ester. A skilled organic chemist would be able to select the appropriate reagents and functional groups for attaching a UV-absorber to the linker moiety of Formula (VII), and would be able to implement the appropriate synthetic protocols. A UV-absorber within formula (IX) can be any UV-absorber generally known in the cosmetics industry as a light-absorbing ingredient of a sunscreen product. In one example, the UV-absorber may be any of the UV-absorber approved by FDA or by EMA for use in sunscreen compositions. Suitable examples of such compounds include salicylic acid, avobenzone, octisalate, oxybenzone, homosalate, octocrylene, aminobenzoic acid, padimate O, phenylbenzimidazole sulfonic acid, cinoxate, dioxybenzone, menthyl anthranilate, octyl methoxycinnamate, dulisobenzone, tolamine salicylate, ecamsule, and similar molecules.

In some embodiments, R³ is selected from any one of the following moieties:

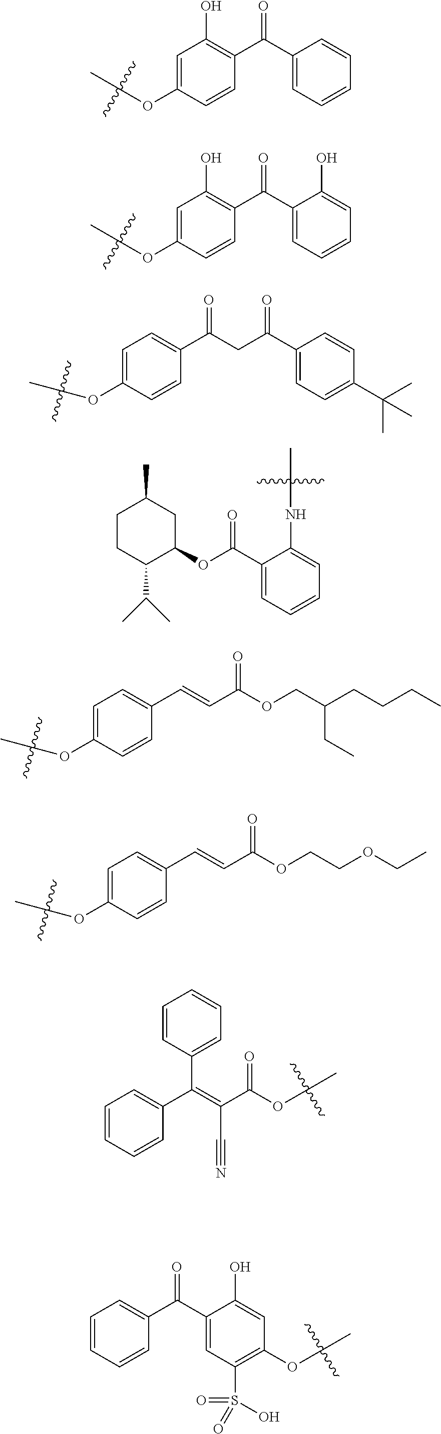

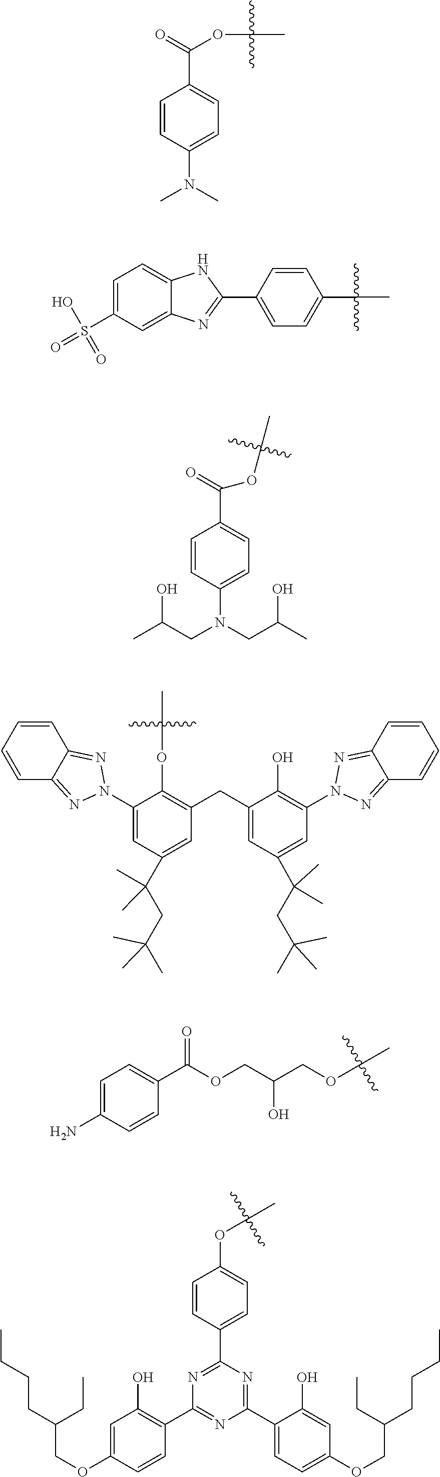 [SMILES: *OC(=O)c1ccc(N(C)C)cc1 ; *c1ccc(-c2nc3cc(S(=O)(=O)O)ccc3[nH]2)cc1 ; *OC(=O)c1ccc(N(CC(C)O)CC(C)O)cc1 ; *Oc1c(Cc2cc(C(C)(C)CC(C)(C)C)cc(-n3nc4ccccc4n3)c2O)cc(C(C)(C)CC(C)(C)C)cc1-n1nc2ccccc2n1 ; *OCC(O)COC(=O)c1ccc(N)cc1 ; *Oc1ccc(-c2nc(-c3ccc(OCC(CC)CCCC)cc3O)nc(-c3ccc(OCC(CC)CCCC)cc3O)n2)cc1]

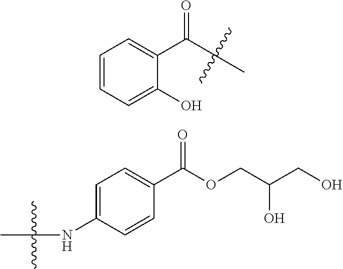

In some embodiments, the compound of Formula (IX) is selected from any one of the following formulae:

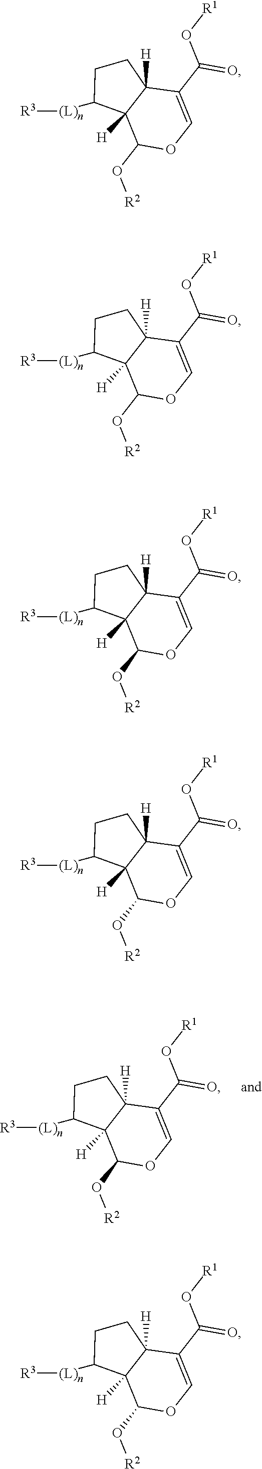

or a pharmaceutically acceptable salt thereof.

In some embodiments, the compound of Formula (IX) is selected from any one of the following compounds:

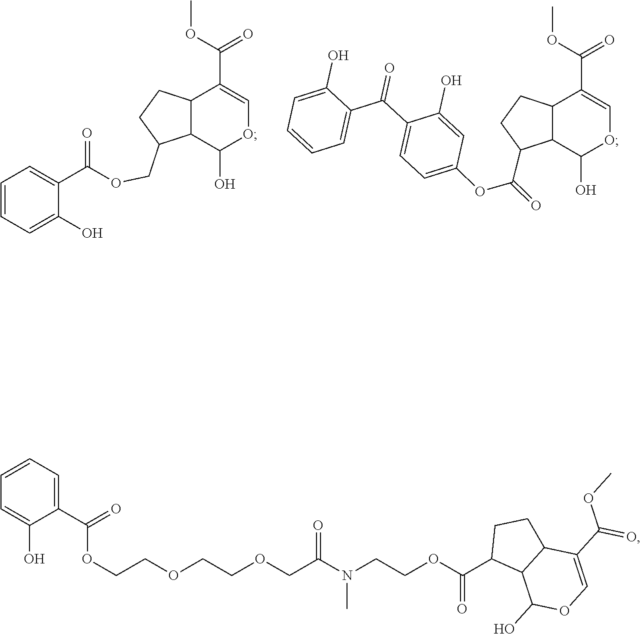

or a pharmaceutically acceptable salt thereof.

### Compounds of Formula (X):

In some embodiments, the present disclosure provides a compound of Formula (X):

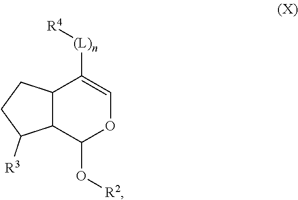

or a pharmaceutically acceptable salt thereof, wherein:
R¹ is a UV-absorbing moiety;
R² is selected from H and an acyl; or
R³ and OR², together with the carbon atoms to which they are attached form a tetrahydrofuran ring, which is optionally substituted with OR^{a1} or oxo;
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is selected from C₁₋₆ alkyl, C₂₋₆ alkenylene, C₂₋₆ alkynylene, C(O)R^{b1}, C(O)NR^{c1}R^{d1}, and C(O)OR^{a1}, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b}, C(O)NR^{c1}R_{d1}, and C(O)OR^{a1};
each R^{a1}, R^{b1}, R^{c1}, and R^{d1} is independently selected from H, C₁₋₆ alkyl, C₁₋₄ haloalkyl, C₂₋₆ alkenyl, and C₂₋₆ alkynyl.

In some embodiments, R² is H. In some embodiments, R² is acyl. In some embodiments, R² is selected from H, formyl, acetyl, propionyl, acrylyl, pivaloyl, and benzoyl.

In some embodiments, R³ and OR², together with the carbon atoms to which they are attached form a tetrahydrofuran ring, which is optionally substituted with OR^{a1} or oxo.

In some embodiments, at least one L is —C₁₋₃ alkylene-. In some embodiments, at least one L is methylene. In some embodiments, at least one L is ethylene.

In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, at least one L is C(═O). In some embodiments, at least one L is NH. In some embodiments, at least one L is O. In some embodiments, at least one L is (—C₁₋₃ alkylene-O—)ₓ. In some embodiments, at least one L is (—O—C₁₋₃ alkylene-)ₓ. In some embodiments, at least one L is (—CH₂CH₂—O—)ₓ. In some embodiments, at least one L is (—O—CH₂CH₂—)ₓ. In some embodiments, x is an integer from 1 to 5. In some embodiments, x is 1, 2, or 3. In some embodiments, (L)ₙ comprises at least one moiety —(C═O)NH—. In some embodiments, (L)ₙ comprises at least one moiety —(C═O)O—. In some embodiments, (L)ₙ comprises at least one moiety —CH₂O—.

In some embodiments, R³ is selected from C₁₋₆ alkyl, C₂₋₆ alkenylene, C₂₋₆ alkynylene, C(O)OR^{a1}, and C(O)R^{b1}, wherein said C₁₋₆ alkyl is optionally substituted with OR^{a1} or NR^{c1}R^{d1};

In some embodiments, R³ is C₁₋₆ alkyl.

In some embodiments, R³ is C₂₋₆ alkenylene.

In some embodiments, R³ is C₂₋₆ alkynylene.

In some embodiments, R³ is C(O)R^{b1}.

In some embodiments, R³ is C(O)NR^{c1}R^{d1}.

In some embodiments, R³ is C(O)OR^{a1}.

In some embodiments, R³ is selected from C(O)R^{b1}, C(O)NR^{c1}R^{d1}, and C(O)OR^{a1}.

In some embodiments, R³ is C₁₋₆ alkyl, optionally substituted with OR^{a1} or NR^{c1}R^{d1}. In some embodiments, R³ is C₁₋₆ alkyl, optionally substituted with a substituent selected from C(O)R^{b1}, C(O)NR^{c1}R^{d1}, and C(O)OR^{a2}.

In some embodiments, R³ is C₁₋₆ alkyl substituted with OR^{a1}.

In some embodiments, R³ is C₁₋₆ alkyl substituted with NR^{c1}R^{d1}.

In some embodiments, R¹ is H. In some embodiments, R¹ is C₁₋₆ alkyl.

In some embodiments, R^{c1} and R^{d1} are each independently selected from H and C₁₋₆ alkyl. In some embodiments, R^{b1} is H. In some embodiments, R^{b1} is C₁₋₆ alkyl.

In some embodiments, the R¹ in Formula (X) is a UV-absorbing moiety as described herein for R³ in Formula (IX).

In some embodiments, the compound of Formula (X) is selected from any one of the following formulae:

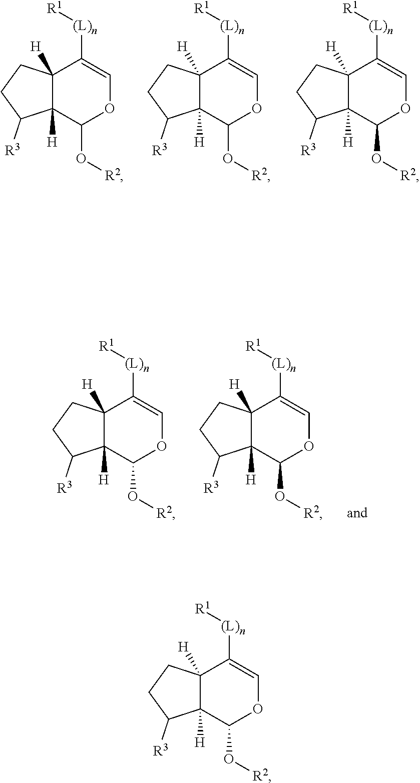

or a pharmaceutically acceptable salt thereof.

In some embodiments, the compound of Formula (X) is selected from any one of the following compounds:

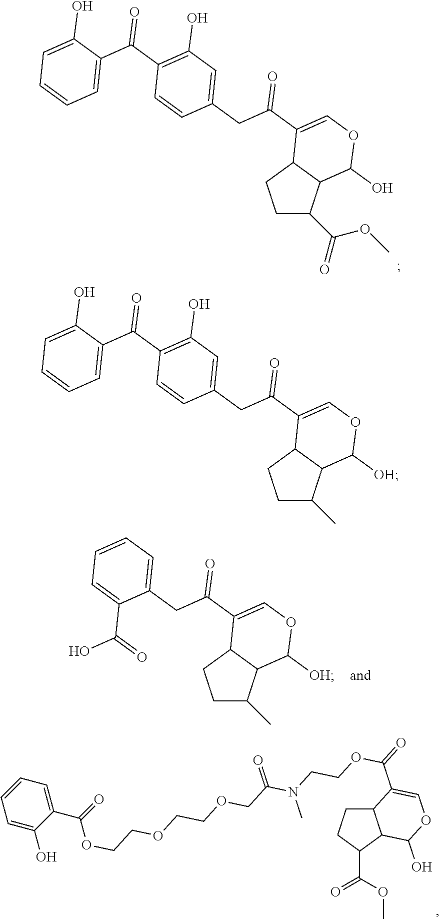

or a pharmaceutically acceptable salt thereof.

### Compounds of Formula (XIII):

In some embodiments, the present disclosure provides a compound of Formula (XIII):

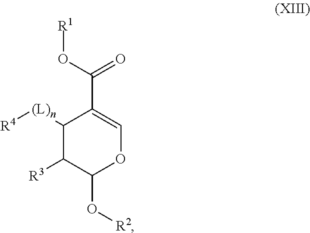

or a pharmaceutically acceptable salt thereof, wherein:
R¹ is selected from H and C₁₋₆ alkyl;
R² is selected from H and an acyl; or
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylen wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁-3 alkyl;
R⁴ is a UV-absorbing moiety;
R³ is selected from C₁₋₆ alkyl, C₂₋₆ alkenylene, C₂₋₆ alkynylene, C(O)R^{b1}, C(O)NR^{c1}R^{d1}, and C(O)OR^{a1};
R^{a1}, R^{b1}, R^{c1} and R^{d1} are each independently selected from H and C₁₋₆ alkyl.

In some embodiments, R¹ is H.

In some embodiments, R¹ is C₁₋₆ alkyl.

In some embodiments, R² is H. In some embodiments, R² is acyl. In some embodiments, R² is selected from H, formyl, acetyl, propionyl, acrylyl, pivaloyl, and benzoyl.

In some embodiments, at least one L is —C₁₋₃ alkylene-. In some embodiments, at least one L is methylene. In some embodiments, at least one L is ethylene.

In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, at least one L is C(═O). In some embodiments, at least one L is NH. In some embodiments, at least one L is O. In some embodiments, at least one L is (—C₁₋₃ alkylene-O—)ₓ. In some embodiments, at least one L is (—O—C₁₋₃ alkylene-)ₓ. In some embodiments, at least one L is (—CH₂CH₂—O—)ₓ. In some embodiments, at least one L is (—O—CH₂CH₂—)ₓ. In some embodiments, x is an integer from 1 to 5. In some embodiments, x is 1, 2, or 3. In some embodiments, (L)ₙ comprises at least one moiety —(C═O)NH—. In some embodiments, (L)ₙ comprises at least one moiety —(C═O)O—. In some embodiments, (L)ₙ comprises at least one moiety —CH₂O—.

In some embodiments, the R⁴ in Formula (XIII) is UV-absorbing moiety as described for R³ in Formula (IX).

In some embodiments, R³ is C₁₋₆ alkyl.

In some embodiments, R³ is C₂₋₆ alkenylene.

In some embodiments, R³ is C₂₋₆ alkynylene.

In some embodiments, R³ is C(O)Rel.

In some embodiments, R³ is C(O)NR^{c1}R^{d1}.

In some embodiments, R³ is C(O)OR^{a1}.

In some embodiments, R³ is selected from C₁₋₆ alkyl, C₂₋₆ alkenylene, and C(O)R^{b1}.

In some embodiments, R^{a1} is H. In some embodiments, R^{a1} is C₁₋₆ alkyl. In some embodiments, R^{b1} is H. In some embodiments, R^{b1} is C₁₋₆ alkyl. In some embodiments, R^{c1} is H. In some embodiments, R^{d1} is C₁₋₆ alkyl. In some embodiments, R^{d1} is H. In some embodiments, R^{d1} is C₁₋₆ alkyl.

In some embodiments, the compound of Formula (XIII) is selected from any one of the following formulae:

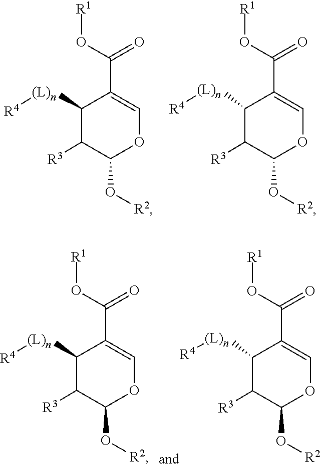

or a pharmaceutically acceptable salt thereof.

In some embodiments, the compound of Formula (XIII) is selected from any one of the following compounds:

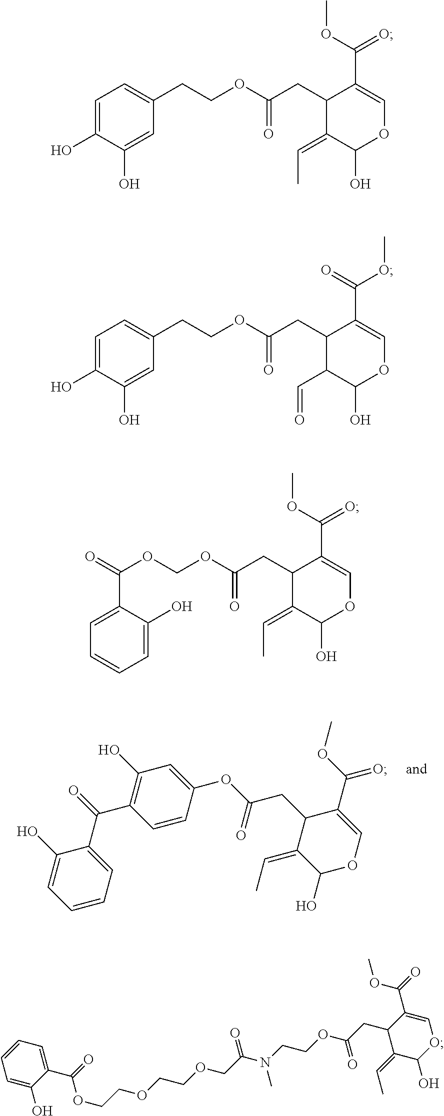 [SMILES: C/C=C1\C(O)OC=C(C(=O)OC)C1CC(=O)OCCc1ccc(O)c(O)c1 ; COC(=O)C1=COC(O)C(C=O)C1CC(=O)OCCc1ccc(O)c(O)c1 ; C/C=C1\C(O)OC=C(C(=O)OC)C1CC(=O)OCOC(=O)c1ccccc1O ; C/C=C1\C(O)OC=C(C(=O)OC)C1CC(=O)Oc1ccc(C(=O)c2ccccc2O)c(O)c1 ; C/C=C1\C(O)OC=C(C(=O)OC)C1CC(=O)OCCN(C)C(=O)COCCOCCOC(=O)c1ccccc1O]

or a pharmaceutically acceptable salt thereof.

### Compound of Formula (XIV):

In some embodiments, the present disclosure provides a compound of Formula (XIV):

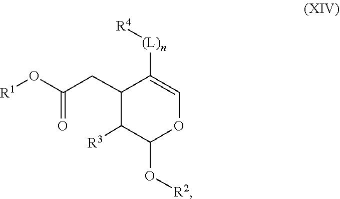

or a pharmaceutically acceptable salt thereof, wherein:
R¹ is selected from H and C₁₋₆ alkyl;
R² is selected from H and an acyl; or
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylen wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁-3 alkyl;
R⁴ is a UV-absorbing moiety;
R³ is selected from C₁₋₆ alkyl, C₂₋₆ alkenylene, C₂₋₆ alkynylene, C(O)R^{b1}, C(O)NR^{c1}R^{d1}, and C(O)OR^{a1}; and
R^{a1}, R^{b1}, R^{c1} and R^{d1} are each independently selected from H and C₁₋₆ alkyl.

In some embodiments, R¹ is H. In some embodiments, R¹ is C₁₋₆ alkyl.

In some embodiments, R² is H. In some embodiments, R² is acyl. In some embodiments, R² is selected from H, formyl, acetyl, propionyl, acrylyl, pivaloyl, and benzoyl.

In some embodiments, at least one L is —C₁₋₃ alkylene-. In some embodiments, at least one L is methylene. In some embodiments, at least one L is ethylene.

In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, at least one L is C(═O). In some embodiments, at least one L is NH. In some embodiments, at least one L is O. In some embodiments, at least one L is (—C₁₋₃ alkylene-O—)ₓ. In some embodiments, at least one L is (—O—C₁₋₃ alkylene-)ₓ. In some embodiments, at least one L is (—CH₂CH₂—O—)ₓ. In some embodiments, at least one L is (—O—CH₂CH₂—)ₓ. In some embodiments, x is an integer from 1 to 5. In some embodiments, x is 1, 2, or 3. In some embodiments, (L)ₙ comprises at least one moiety —(C═O)NH—. In some embodiments, (L)ₙ comprises at least one moiety —(C═O)O—. In some embodiments, (L)ₙ comprises at least one moiety —CH₂O—.

In some embodiments, the R⁴ in Formula (XIV) is UV-absorbing moiety as described for R³ in Formula (IX).

In some embodiments, R³ is C₁₋₆ alkyl.

In some embodiments, R³ is C₂₋₆ alkenylene.

In some embodiments, R³ is C₂₋₆ alkynylene.

In some embodiments, R³ is C(O)R^{b1}.

In some embodiments, R³ is C(O)NR^{c1}R^{d1}.

In some embodiments, R³ is C(O)OR^{a1}.

In some embodiments, R³ is selected from C₁₋₆ alkyl, C₂₋₆ alkenylene, and C(O)R^{b1}.

In some embodiments, R^{a1} is H. In some embodiments, R^{a1} is C₁₋₆ alkyl. In some embodiments, R^{b1} is H. In some embodiments, R^{b1} is C₁₋₆ alkyl. In some embodiments, R^{c1} is H. In some embodiments, R^{c1} is C₁₋₆ alkyl. In some embodiments, R^{d1} is H. In some embodiments, R^{d1} is C₁₋₆ alkyl.

In some embodiments, the compound of Formula (XIV) is selected from any one of the following formulae:

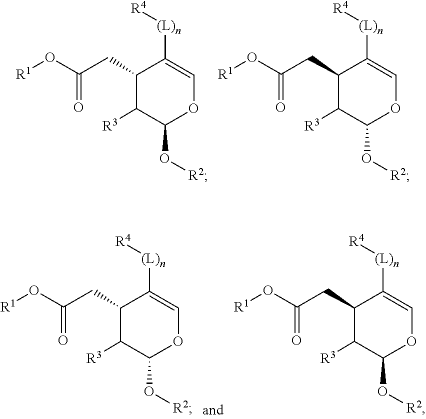

or a pharmaceutically acceptable salt thereof.

In some embodiments, the compound of Formula (XIV) is selected from any one of the following compounds:

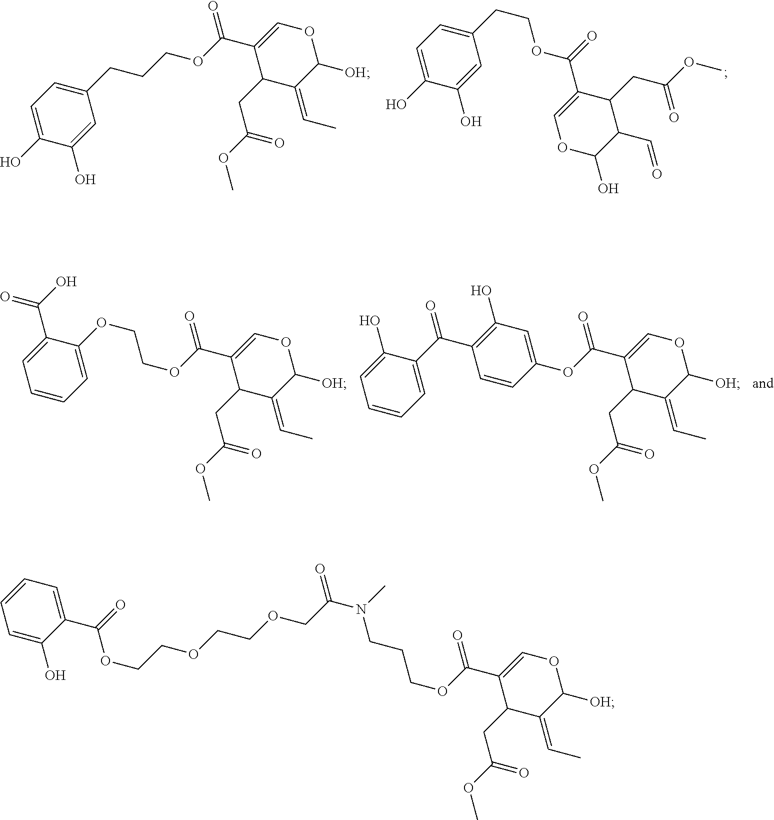

or a pharmaceutically acceptable salt thereof.

In some embodiments, the compound of Formula (IX), Formula (X), Formula (XIII), or Formula (XIV) is stable (i.e., retains its original chemical structure) after application to the subject's skin for a period of time from about 30 min to about 2 hours, from about 1 hour to about 2 hours, or from about 1 hour to about 3 hours, from about 1 h to about 1 year, from about 1 day to about 6 months, from about 1 day to about 3 months, or from about 1 month to about 6 months. In some embodiments, the compound remains stable and bound to the subject's skin for a about 1 week, about 2 weeks, about 3 weeks, or about 4 weeks.

### Topical Compositions and Methods of Using the Compounds

The present disclosure also provides compositions comprising a compound of any one of the formulae disclosed herein, or a pharmaceutically acceptable salt thereof, for use in a tropical application. Topical administration in this sense refers to any local (i.e. not systemic) administration, whether through ointments, gels, creams, lotions, or other similar formulations, of the compounds or compositions of the present disclosure, including administration directly to the external epidermis or dermis a subject, but excluding oral, rectal, intrapulmonary and intranasal administration. The present disclosure further pertains to the compounds or compositions of the present disclosure for use as a cosmetic. In the present application, a cosmetic or a cosmetic use means that the composition is suitable for external use (i.e. extracorporal use, e.g. not ingested) and is in particular suitable for application to the skin or hair.

Cosmetic uses include the application of the compounds or compositions of the present disclosure, in particular those comprising a dye moiety, for decorative purposes, for instance as semi-permanent tattoo or semi-permanent make-up, lipstick, eye shadow, rouge, concealer of blemishes and/or scars, skin toner or a decorative skin whitener, hair colorant.

Further cosmetic uses include the application of the compounds or compositions of the present disclosure, in particular those comprising a UVa and/or a UVB absorbing moiety, as a sunscreen on the skin of a human or mammalian subject.

Still further cosmetic uses include the application of the compounds or compositions of the present disclosure as skin brighteners which e.g. provide a healthy glow to the skin. Particularly suitable for this purpose are compounds or compositions of the present disclosure comprising a UVa and/or a UVB absorbing moiety which is also a fluorescent moiety, i.e. which absorbs light in the UVA and/or UVB spectrum (as defined elsewhere herein) and reemits light in the visible spectrum (as also defined elsewhere herein). Such brightening moieties are well-known in the art and include stilbene moieties, e.g. moieties of 4,4′-diamino-2,2′-stilbenedisulfonic acid, and benzoxazolyl moieties such as 4′-bis(benzoxazolyl)-cis-stilbene and 2,5-bis(benzoxazol-2-yl)thiophene.

Generally, the aforementioned compositions can be formulated in any form known in the art for cosmetic (topical) administration. Hence, the composition can be applied in any topical form, such as in the form of aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic/sunscreen and skin care formulation. The composition can also be water-resistant (e.g., water proof).

The compositions of this disclosure may contain any one of the compounds described herein in the range of 0.005 wt.-% to 99 wt.-% with the balance made up from the suitable excipients. The contemplated compositions may contain 0.001 wt.-%-99 wt.-% of any one of the compounds provided herein, in one embodiment 0.1-95 wt.-%, in another embodiment 75-85 wt.-%, in a further embodiment 20-80 wt.-%, wherein the balance may be made up of any excipient described herein, or any combination of these excipients.

The (topical) compositions can be in an emulsion, cream, or lotion form. Topical administration of the pharmaceutical compositions of the present application is especially useful when the desired treatment involves areas of skin readily accessible by topical application (e.g., arms, chest, legs, neck, face, etc.). In some embodiments, the topical composition comprises a combination of any one of the compounds disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, additional conventional sunscreen actives (non-limiting examples of such sunscreens include titanium oxide and zinc oxide), surfactants/detergent cleansing agents, penetration enhancers, and thickeners. In particular, the composition may contain any acceptable excipient that is approved by FDA or EMA for such use. Examples of acceptable inactive excipients include retinyl palmitate, parabens, PEGs, fragrance, BHT, phthalates, phenoxyethanol, coconut oil, aloe vera, sunflower seed oil, calendula, almond oil, starch, gelatin, hemicellulose, arabinogalactan, agar, glycerol, lactic acid, propylene glycol, polyethylene glycol, polyvinylpyrrolidone, acrylates, acrylamides, copolymers of the foregoing, an amino acid such as arginine, alanine, or asparagine, ascorbic acid, and citric acid.

Besides their cosmetical use, the compounds and topical compositions are also suitable for use in medical indications, for instance for preventing or ameliorating cancers of the skin such as melanoma. The compounds and topical compositions are also useful in preventing sunburn in farm animals which are at least periodically kept outdoors and, thus, are exposed to sunlight. This in particular applies to compounds and topical compositions of the present disclosure in which a moiety as defined for the respective embodiment is a UVA and/or UVB absorbing moiety or in compounds in which a dedicated dye moiety is absent. The compounds and topical compositions of the present disclosure in which a moiety as defined for the respective embodiment is a dye moiety are particularly useful in a (non-therapeutic) method of semi-permanently tattooing skin of a subject, e.g. a human or a farm animal.

### Other Uses

The compounds of the present disclosure are also useful in formulations other than topical compositions. For instance, the compounds can be provided in an injectable composition, in particular an injectable composition which allows application of the compounds in deeper layers of the skin, as is e.g. done in conventional tattooing. Accordingly, another aspect, there is also provided an injectable composition comprising any of the compounds disclosed herein, in particular a compound comprising a dye moiety.

### Definitions

As used herein, the term “about” means “approximately” (e.g., plus or minus approximately 10% of the indicated value). For example, “about 20” means or includes amounts from 18 to and including 22.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the term “carboxy” refers to a —C(O)OH group.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons.

Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “amino” refers to a group of formula —NH₂.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, a “salt” or “pharmaceutically acceptable salt” of a compound of any one of the formulae disclosed herein is formed between an acid and a basic group of the compound, such as an amino functional group, or a base and an acidic group of the compound, such as a carboxyl functional group. According to another embodiment, the compound is a pharmaceutically acceptable acid addition salt. In some embodiments, acids commonly employed to form pharmaceutically acceptable salts of the compounds of any one of the formulae include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, funarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In some embodiments, bases commonly employed to form pharmaceutically acceptable salts of the compounds of any one of the formulae disclosed herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds of any one of the formulae disclosed herein, or salts thereof, are substantially isolated.

As used herein, the terms “individual” or “subject” are used interchangeably, and refer to any animal, including mammals, preferably mice, rats, other rodents, rabbits, dogs, cats, swine, cattle, sheep, horses, or primates, and most preferably humans. In one embodiment, the term “subject” refers to a species of plant kingdom. For example, the term “subject” may refer to plants, such as flowering plants, or fruit (e.g., grapes). In this aspect, the term encompasses the leaves as well as the fruit itself.

The terms “protecting group” and “protective group” refer to a moiety that reversibly chemically modifies a functional group in order to obtain chemoselectivity or in order to reduce degradation in one or more subsequent chemical reactions. Suitable protecting groups are well known in the art (see, e.g., Greene and Wuts, Protective Groups in Organic Synthesis. 3rd Ed., John Wiley & Sons, New York, N.Y., 1999, which is incorporated herein by reference in its entirety).

As used herein, a “colorant” is used interchangeably with “dye” and “pigment,” and refers to a substance that changes the color of reflected or transmitted light as the result of wavelength-selective absorption.

As used herein, a “temporary colorant” refers to a colorant that sits on top of the skin or, if it penetrates the skin, can diffuse out of the skin or can be washed off by, for example, water, soap, and/or isopropanol.

As used herein, “color” refers to wavelengths of electromagnetic radiation visible to the human eye.

### EXAMPLES

All starting materials/reagents/solvents were obtained from Sigma Aldrich, Thermo Fisher, VWR, TCI Chemicals, or Oakwood chemicals and used without purification. Genipin was supplied by Herb-Sun Biotechnology.

### Example 1—Preparation of Genipin Derivative Compound methyl 1-hydroxy-7-methyl-1,4a,5,6,7,7a-hexahydrcyclopenta[c]pyran-4-carboxylate (Compound 1)

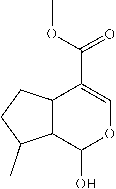

Genipin (methyl 1-hydroxy-7-(hydroxymethyl)-1,4a,5,7a-tetrahydrocyclopenta[c]pyran-4-carboxylate) (1 equivalent) was added to a Schlenk flask along with 10% Pd/C (10 wt. %). The flask was cycled between vacuum and nitrogen gas three times. In a separate flask, nitrogen was bubbled into methanol for 15 minutes. Once complete, methanol was added slowly (0.05 M) to the Schlenk flask under flow of nitrogen. The Schlenk flask was put under vacuum and backfilled with H₂ via balloon. The reaction was warmed to 50° C. and monitored by TLC until complete conversion of starting material. The crude reaction mixture was filtered through a pad of celite and flushed with dichloromethane. The filtered solution was concentrated and isolated by flash column chromatography as a mixture of diastereomers. ¹H NMR (300 MHz, CDCl₃) δ 7.42 (d, J=1.3 Hz, 1H), 4.91 (t, J=6.3 Hz, 1H), 3.71 (s, 3H), 2.89 (m, 1H), 2.35-2.14 (m, 1H), 2.11-1.94 (m, 1H), 1.94-1.78 (m, 1H), 1.64 (m, 1H), 1.35-1.15 (m, 2H), 1.11 (d, J=6.7 Hz, 3H). HRMS (DART+): calculated for C₁₁H₁₇O₄ [M+H+]: 213.1121 m/z, found: 213.1124 m/z.

### Example 2—Preparation of a Hydrogenated Genipin Derivative Compound methyl 1-hydroxy-7-(hydroxymethyl)-1,4a,5,6,7,7a-hexahydrocyclopenta[c]pyran-4-carboxylate (Compound 2)

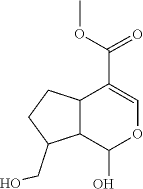 [SMILES: COC(=O)C1=COC(O)C2C(CO)CCC12]

### Step 1—Synthesis of methyl 1-((tert-butyldimethylsilyl)oxy)-7-(((tert-butyldimethylsilyl)oxy)methyl)-1,4a,5,7a-tetrahydrocyclopenta[c]pyran-4-carboxylate

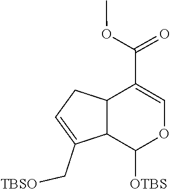 [SMILES: COC(=O)C1=COC(O[Si](C)(C)C(C)(C)C)C2C(CO[Si](C)(C)C(C)(C)C)=CCC12]

Genipin (1 equivalent) was added to an oven-dried round bottom flask along with tert-butyldimethylsilyl chloride (2.4 equivalents) and imidazole (5 equivalents). The reagents were then dissolved in dimethylformamide (0.3 M) and stirred at room temperature for 4 days. After completion, the crude reaction mixture was added to a separatory funnel with ethyl acetate and brine. Organic phase was washed with brine three times. The combined aqueous layer was extracted with ethyl acetate two times. The combined organic phase was dried with magnesium sulfate and concentrated using a rotary evaporator. The title compound was isolated via flash column chromatography using a gradient of ethyl acetate and hexane.

### Step 2—Synthesis of methyl 1-((tert-butyldimethylsilyl)oxy)-7-(((tert-butyldimethylsilyl)oxy)methyl)-1,4a,5,6,7,7a-hexahydrocyclopenta[c]pyran-4-carboxylate

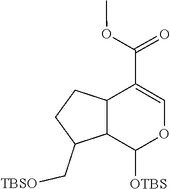 [SMILES: COC(=O)C1=COC(O[Si](C)(C)C(C)(C)C)C2C(CO[Si](C)(C)C(C)(C)C)CCC12]

The product of step 1 (1 equivalent) was added to a Schlenk flask along with 10% Pd/C (10 wt. %). The flask was cycled between vacuum and nitrogen gas three times. In a separate flask, nitrogen was bubbled into methanol for 15 minutes. Once complete, methanol was added slowly (0.05 M) to the Schlenk flask under flow of nitrogen. The Schlenk flask was put under vacuum and backfilled with H₂ via balloon.

The reaction was warmed to 50° C. and monitored by TLC until complete conversion of starting material. The crude reaction mixture was filtered through a pad of celite and flushed with dichloromethane. The filtered solution was concentrated and isolated by flash column chromatography as a mixture of diastereomers.

### Step 3—Synthesis of methyl 1-hydroxy-7-(hydroxymethyl)-1,4a,5,6,7,7a-hexahydrocyclopenta[c]pyran-4-carboxylate

The product of step 2 (1 equivalent) was added to an oven-dried flask and dissolved in THF (0.04 M). To this was added TBAF (1 equivalent from a 1 M solution in THF), and the reaction was stirred for 1 hour. The crude reaction mixture was concentrated, and the desired compound was isolated via flash column chromatography. ¹H NMR (400 MHz, MeOD) δ 7.48 (s, 1H), 3.70 (s, 3H), 3.64-3.56 (m, 1H), 3.51 (m, 1H), 2.80-2.70 (m, 1H), 2.27-2.07 (m, 2H), 2.00-1.64 (m, 3H), 1.50-1.19 (m, 3H). HRMS (DART+): calculated for C₁₁H₁₇O₅ [M+H+]: 229.1070 m/z, found: 229.1066 m/z.

### Example 3—Preparation of Genipin Derivative Compound methyl 7-formyl-1-hydroxy-1,4a,5,6,7,7a-hexahydrocyclopenta[c]pyran-4-carboxylate (compound 6) and genipin derivative compound methyl 3-hydroxy-1,2a,2a1,3,4a,7a-hexahydro-2H-4,5-dioxacyclopenta[cd]indene-7-carboxylate (Compound 7)

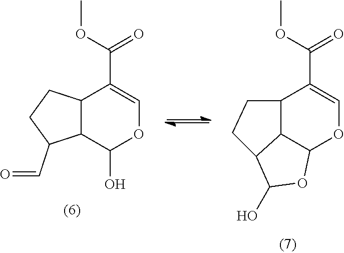 [SMILES: COC(=O)C1=COC(O)C2C(C=O)CCC12 ; COC(=O)C1=COC2OC(O)C3CCC1C23]

### Step 1—methyl 7-(hydroxymethyl)-1-methoxy-1,4a,5,7a-tetrahydrocyclopenta[c]pyran-4-carboxylate

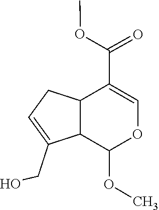 [SMILES: COC(=O)C1=COC(OC)C2C(CO)=CCC12]

Genipin (1 equivalent) was added to a round bottom flask and dissolved with methanol (0.05 M). Para-toluenesulfonic acid (0.3 equivalent) was added to the stirring solution of genipin and allowed to react at room temperature until complete conversion of starting material. Once complete, the crude material was concentrated using rotary evaporation and filtered through a plug of silica. The crude material was flushed with dichloromethane and concentrated. The title compound was isolated via flash column chromatography.

### Step 2—methyl 7-formyl-J-methoxy-1,4a,5,7a-tetrahydrocyclopenta[c]pyran-4-carboxylate

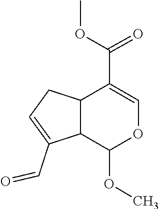 [SMILES: COC(=O)C1=COC(OC)C2C(C=O)=CCC12]

To a stirring solution o e product o step 1 (1 equivalent) in dichloromethane, was added Dess-Martin periodinane (DMP) (1.2 equivalent). The reaction was allowed to stir for 24 hours. Once complete, a saturated solution of NaHCO₃ and Na₂S₂O₃ were added sequentially to the reaction mixture and stirred for 30 minutes. The biphasic mixture was added to a separatory funnel and extracted with dichloromethane/water (3×) followed by brine. The combined organic layer was dried with MgSO₄ and concentrated under reduced pressure. The crude material was dry loaded onto silica and purified by flash chromatography (hexane:ethyl acetate) to give the title compound.

### Step 3—methyl 7-formyl-1-methoxy-1,4a,5,6,7,7a-hexahydrocyclopenta[c]pyran-4-carboxylate

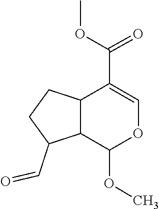 [SMILES: COC(=O)C1=COC(OC)C2C(C=O)CCC12]

Product of step 2 (1 equivalent) was added to a Schlenk flask along with 10% Pd/C (10 wt. %). The flask was cycled between vacuum and nitrogen gas three times. In a separate flask, nitrogen was bubbled into methanol for 15 minutes. Once complete, methanol was added slowly (0.05 M) to the Schlenk flask under flow of nitrogen. The Schlenk flask was put under vacuum and backfilled with H₂ via balloon. The reaction was warmed to 50° C. and monitored by TLC until complete conversion of starting material. The crude reaction mixture was filtered through a pad of celite and flushed with dichloromethane. The filtered solution was concentrated and isolated by flash column chromatography as a mixture of diastereomers (the title compound).

### Step 4—Synthesis of Compound 6 and 7

Product of step 3 was dissolved in acetic acid/1 M HCl/THF in a 3:2:5 ratio at an overall concentration of 0.07 M. The solution was stirred overnight at 60° C. Once complete, the solution was poured into a separatory funnel with water and ethyl acetate. The combined ethyl acetate extract was dried with sodium sulfate and concentrated under reduced pressure. The desired compound (as a 15:85 mixture of aldehyde compound 6/acetal compound 7) was isolated via flash column chromatography (hexane:ethyl acetate). ¹H NMR (400 MHz, CDCl₃) δ 9.79 (s, 0.05H), 9.97 (s, 0.11H) 7.56-7.44 (m, 1H), 6.07 (d, J=5.9 Hz, 0.05H), 5.90-5.82 (m, 0.55H), 5.74 (d, J=5.0 Hz, 0.11H), 5.36 (d, J=4.9 Hz, 0.05H), 5.11 (d, J=1.6 Hz, 0.55H), 5.03 (d, J=1.5 Hz, 0.11H), 4.91 (d, J=7.5 Hz, 0.15H), 3.74 (m, 3H), 3.61-3.55 (m, 0.05H), 3.38 (t, J=7.4 Hz, 0.05H), 3.02 (m, 0.75H), 2.94-2.78 (m, 1H), 2.76-2.58 (m, 1.4H), 2.56-2.43 (m, 0.20H), 2.39-2.22 (m, 1H), 2.06 (m, 0.25H), 1.93-1.80 (m, 1H), 1.72 (m, 1H), 1.44 (m, 0.11H), 1.15 (m, 0.8H). HRMS (DART+): calculated for C₁₁H₁₅O₅ [M+H+]: 227.0914 m/z, found: 227.0913 m/z.

### Example 4—Synthesis of 1-hydroxy-4-(methoxycarbonyl)-1,4a,5,6,7,7a-hexahydrocyclopenta[c]pyran-7-carboxylic acid (compound 8) and methyl 3-oxo-1,2a,2a,3,4a,7a-hexahydro-2H-4,5-dioxacyclopenta[cd]indene-7-carboxylate (Compound 9)

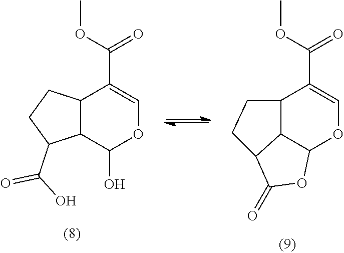 [SMILES: COC(=O)C1=COC(O)C2C(C(=O)O)CCC12 ; COC(=O)C1=COC2OC(=O)C3CCC1C23]

### Step 1—Synthesis of 1-methoxy-4-(methoxycarbonyl)-1,4a,5,6,7,7a-hexahydrocyclopenta[c]pyran-7-carboxylicacid

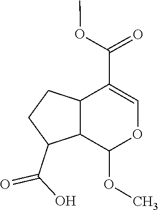 [SMILES: COC(=O)C1=COC(OC)C2C(C(=O)O)CCC12]

The product of step 3 of example 3 was dissolved in dimethylformamide (0.2 M) and stirred with 4 equivalents of potassium peroxymonosulfate for 48 hours. The crude material was added to a separatory funnel with ethyl acetate and brine. The combined ethyl acetate was dried with sodium sulfate and concentrated before isolation via flash column chromatography to give the title compound.

### Step 2—Synthesis of Compound 8 and compound 9

The product of step 1 was dissolved in acetic acid/1 M HCl/MHF in a 3:2:5 ratio at an overall concentration of 0.07 M. The solution was stirred overnight at 60° C. Once complete, the solution was poured into a separatory funnel with water and ethyl acetate. The combined ethyl acetate extract was dried with sodium sulfate and concentrated. The title compound was isolated via flash column chromatography (hexane:ethyl acetate) to give the desired compound below. ¹H NMR (400 MHz, CDCl₃) δ 7.47 (d, J=1.2 Hz, 1H), 6.05 (d, J=5.9 Hz, 1H), 3.75 (s, 3H), 3.41-3.32 (m, 1H), 2.97 (ddd, J=10.6, 7.7, 5.9 Hz, 1H), 2.93-2.81 (m, 1H), 2.54-2.43 (m, 1H), 2.26 (dd, J=12.9, 6.0 Hz, 1H), 1.76 (tdd, J=13.2, 7.1, 6.2 Hz, 1H), 1.10 (tdd, J=13.2, 11.4, 6.0 Hz, 1H). ¹³C NMR (101 MHz, CDCl₃) δ 175.9, 167.1, 148.5, 110.0, 96.9, 51.7, 48.8, 37.5, 33.4, 31.7, 29.0. HRMS (DART+): calculated for C₁₁H₁₃O₅ [M+H+]: 225.0758 m/z, found: 225.0751 m/z.

### Example 5—Synthesis of methyl (E)-1-hydroxy-7-(((4-(phenyldiazenyl)benzoyl)oxy)methyl)-1,4a,5,6,7,7a-hexahydrocyclopenta[c]pyran-4-carboxylate

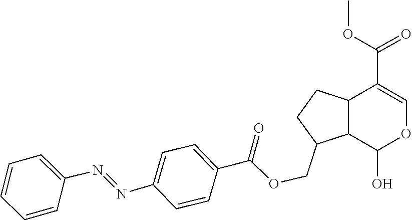 [SMILES: COC(=O)C1=COC(O)C2C(COC(=O)c3ccc(/N=N/c4ccccc4)cc3)CCC12]

### Step 1—Synthesis of methyl 7-(hydroxymethyl)-1-methoxy-1,4a,5,6,7,7a-hexahydrocyclopenta[c]pyran-4-carboxylate

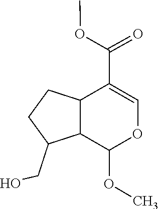 [SMILES: COC(=O)C1=COC(OC)C2C(CO)CCC12]

The compound obtained in step 3 of example 3 (1 equivalent) was dissolved in methanol (0.27 M) and stirred at 0° C. To this, was added sodium borohydride (1.5 equivalents). The reaction was stirred for 30 minutes as it warmed to room temperature. Once complete, the crude reaction mixture was diluted with saturated NH₄Cl and extracted with ethyl acetate (3×) in a separatory funnel. The combined organic phase was dried with sodium sulfate and concentrated under reduced pressure. Title compound was isolated via flash column chromatography.

### Step 2—synthesis of methyl (E)-1-methoxy-7-(((4-(phenyldiazenyl)benzoyl)oxy)methyl)-1,4a,5,6,7,7a-hexahydrocyclopenta[c]pyran-4-carboxylate

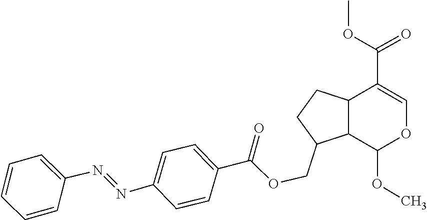 [SMILES: COC(=O)C1=COC(OC)C2C(COC(=O)c3ccc(/N=N/c4ccccc4)cc3)CCC12]

The compound obtained in step 1 (1 equivalent) was dissolved in dichloromethane/pyridine (1:1 v/v) (0.2 M) and cooled to 0° C. 4-(phenylazo)benzoyl chloride (1.2 equivalents) was added dropwise at 0° C. and allowed to react overnight while warming to room temperature. The crude reaction mixture was quenched with saturated sodium bicarbonate and extracted in a separatory funnel with dichloromethane and water. The combined DCM layer was dried with sodium sulfate, concentrated under reduced pressure and isolated via flash column chromatography to give the title compound.

### Step 3—Synthesis of the Title Compound of this Example

The product of step 2 was dissolved in acetic acid/1M HCl/MHF in a 3:2:5 ratio at an overall concentration of 0.07 M. The solution was stirred overnight at 60° C. Once complete, the solution was poured into a separatory funnel with water and ethyl acetate. The combined ethyl acetate extract was dried with sodium sulfate and concentrated under reduced pressure. The title compound was isolated via flash column chromatography (hexane:ethyl acetate) to give the desired compound. ¹H NMR (300 MHz, CDCl₃) δ 8.22-8.15 (m, 2H), 7.99-7.91 (m, 4H), 7.59-7.50 (m, 3H), 7.45 (m, 1H), 5.66 (t, J=3.7 Hz, 0.3H), 5.19-5.09 (m, 0.65H), 4.69 (dd, J=11.2, 7.3 Hz, 0.65H), 4.56 (d, J=8.0 Hz, 0.6H), 4.46 (dd, J=11.3, 7.4 Hz, 0.6H), 3.74 (s, 3H), 3.69 (m, 1H), 3.63-3.55 (m, 2H), 3.08 (m, 0.3H), 2.98 (m, 0.7H), 2.79 (m, 1H), 2.45-2.18 (m, 2H), 2.12-2.03 (m, 0.6H), 1.95-1.81 (m, 2H), 1.79-1.59 (m, 3H), 1.55-1.41 (m, 0.6H). HRMS (DART+): calculated for C₂₄H₂₅N₂O₆ [M+H+]: 437.1707 m/z, found: 437.1714 m/z.

### Example 6—Color Properties of Hydrogenated Genipin

A hydrogenated genipin derivative (compound 1) was conjugated with lysine to obtain compound 1-lysine conjugate:

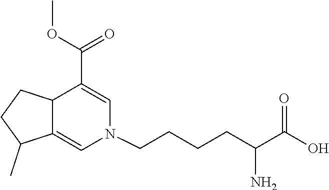 [SMILES: COC(=O)C1=CN(CCCCC(N)C(=O)O)C=C2C(C)CCC12]

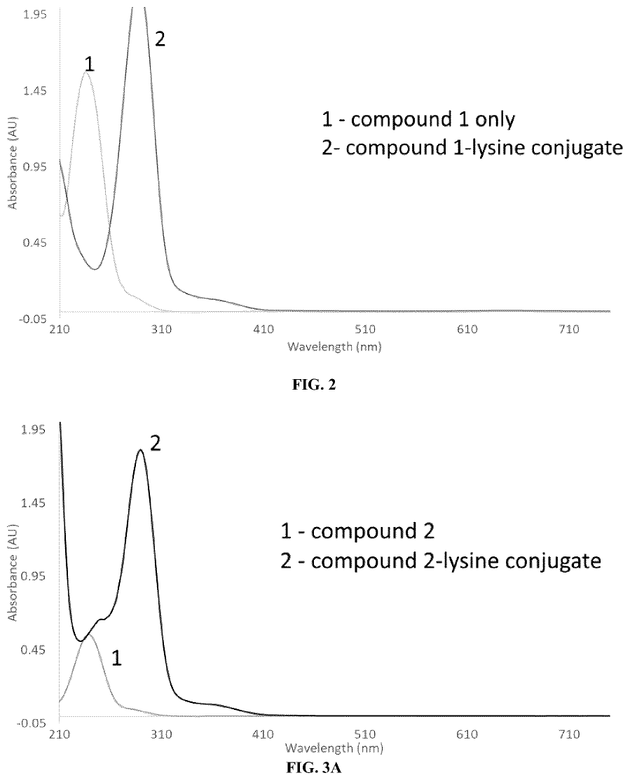

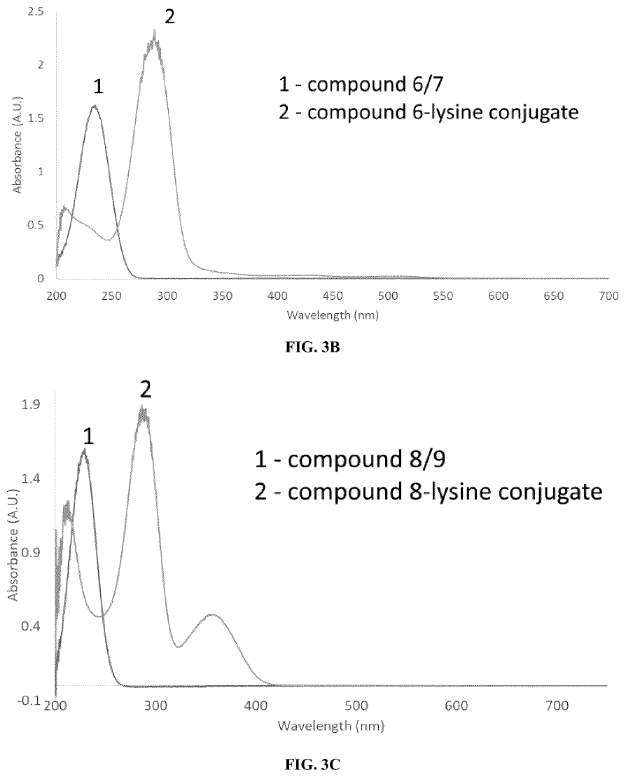

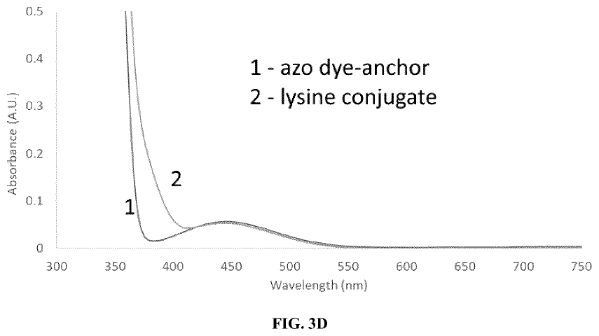

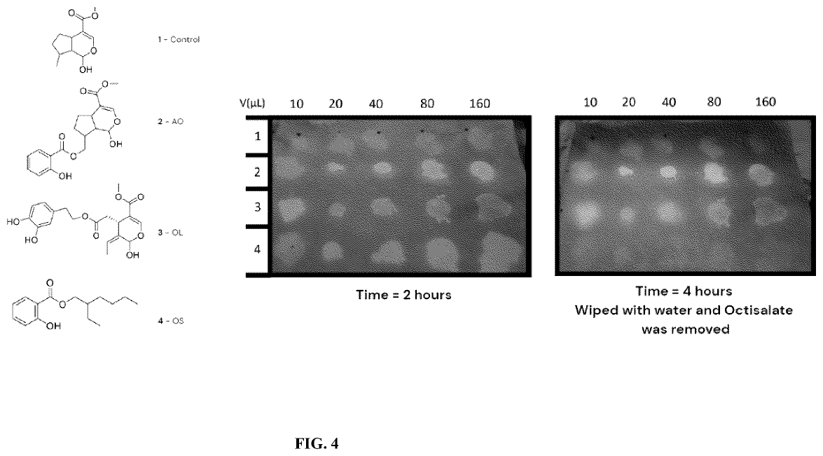

As shown in FIG. 2, reacting the anchor compound 1 with the amino acid to mimic skin binding results in a small shift in the compound's strong UV-absorption (absorbance at wavelengths below 410 nm), but no visible color (no absorbance at wavelengths above 410 nm). FIG. 3A shows that reacting the anchor compound 2 with lysine to mimic skin binding results in strong UV-light absorption and no visible color. FIG. 3B shows that reacting the anchor compound 6/7 with lysine to mimic skin binding results in UV-light absorption and no visible color. FIG. 3C shows that reacting the anchor compound 8/9 with lysine to mimic skin binding results in UV-light absorption and no visible color.

The azo-dye compound of example 5 was conjugated with lysine to obtain a conjugate:

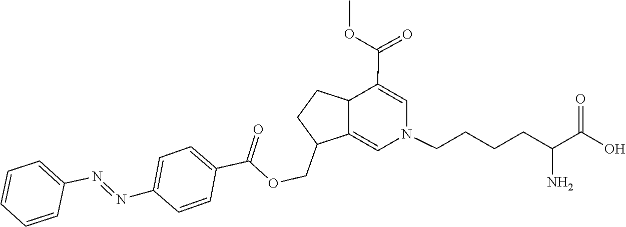 [SMILES: COC(=O)C1=CN(CCCCC(N)C(=O)O)C=C2C(COC(=O)c3ccc(/N=N/c4ccccc4)cc3)CCC12]

As shown in FIG. 3D, reacting the dye conjugate of the anchor compound with the amino acid to mimic skin binding results does not alter absorbance properties of the due compound prior to conjugation with lysine.

### Example 7—methyl 1-hydroxy-7-(((2-hydroxybenzoyl)oxy)methyl)-1,4a,5,6,7,7a-hexahydrocyclopenta[c]pyran-4-carboxylate

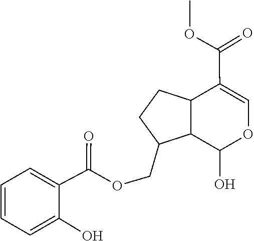 [SMILES: COC(=O)C1=COC(O)C2C(COC(=O)c3ccccc3O)CCC12]

### Step 1—Synthesis of methyl 7-(hydroxymethyl)-1-methoxy-1,4a,5,6,7,7a-hexahydrocyclopenta[c]pyran-4-carboxylate

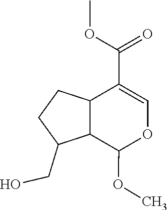 [SMILES: COC(=O)C1=COC(OC)C2C(CO)CCC12]

The compound obtained in step 3 of example 3 (1 equivalent) was dissolved in methanol (0.27 M) and stirred at 0° C. To this, was added sodium borohydride (1.5 equivalents). The reaction was stirred for 30 minutes as it warmed to room temperature. Once complete, the crude reaction mixture was diluted with saturated NH₄Cl and extracted with ethyl acetate (3×) in a separatory funnel. The combined organic phase was dried with sodium sulfate and concentrated under reduced pressure. Title compound was isolated via flash column chromatography.

### Step 2—Synthesis of methyl 7-(((2-acetoxybenzoyl)oxy)methyl)-1-methoxy-1,4a,5,6,7,7a-hexahydrocyclopenta[c]pyran-4-carboxylate

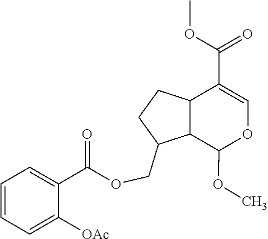 [SMILES: COC(=O)C1=COC(OC)C2C(COC(=O)c3ccccc3OC(C)=O)CCC12]

The product of step 1 (1 equivalent) was dissolved in dichloromethane/pyridine (1:1 v/v) (0.2 M) and cooled to 0° C. O-acetylsalicyloyl chloride (1.2 equivalents) was added dropwise at 0° C. and allowed to react overnight while warming to room temperature. The crude reaction mixture was quenched with saturated sodium bicarbonate and extracted in a separatory funnel with dichloromethane and water. The combined DCM layer was dried with sodium sulfate, concentrated under reduced pressure and isolated via flash column chromatography to give the title compound.

### Step 3—Synthesis of the Title Compound of this Example

The product of step 2 was dissolved in acetic acid/1 M HCl/MHF in a 3:2:5 ratio at an overall concentration of 0.07 M. The solution was stirred overnight at 60 15° C. Once complete, the solution was poured into a separatory funnel with water and ethyl acetate. The combined ethyl acetate extract was dried with sodium sulfate and concentrated under reduced pressure. The title compound was isolated via flash column chromatography (hexane:ethyl acetate) to give the desired compound. ¹H NMR (400 MHz, CDCl₃) δ 10.79 (s, 0.25H), 10.77 (s, 0.6H), 7.84 (m, 1H), 7.49-7.45 (m, 2H), 7.01 (m, 1H), 6.91 (m, 1H), 5.65 (t, J=3.6 Hz, 0.25H), 5.15 (dd, J=8.5, 6.5 Hz, 0.7H), 4.67 (dd, J=11.1, 7.2 Hz, 0.7H), 4.58 (d, J=8.2 Hz, 0.5H), 4.48 (dd, J=11.2, 7.6 Hz, 0.7H), 3.76 (m, 3H), 3.34 (m, 0.7H), 3.00 (m, 1H), 2.95-2.68 (m, 1H), 2.46-2.30 (m, 1H), 2.26 (m, 0.7H), 2.18-1.89 (m, 1H), 1.83-1.43 (m, 2H). HRMS (DART+): calculated for C₁₅H₂₄NO₇ [M+NH+]: 366.1547 m/z, found: 366.1550 m/z.

### Example 8—Oleuropein Derivatives

The following compound (OL) was prepared by de-glycosylating naturally occurring compound oleuropein:

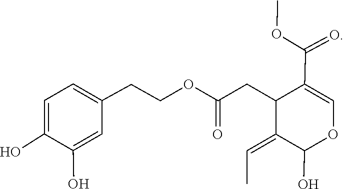 [SMILES: C/C=C1\C(O)OC=C(C(=O)OC)C1CC(=O)OCCc1ccc(O)c(O)c1]

The following compound was prepared as a mixture of isomers by rearrangement of the oleuropein aglycone in solution:

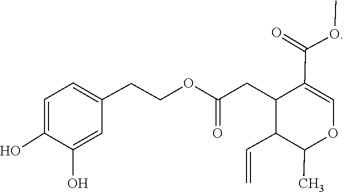 [SMILES: C=CC1C(C)OC=C(C(=O)OC)C1CC(=O)OCCc1ccc(O)c(O)c1]

### Example 9—Conjugation with Lysine

The compound of Example 7 was conjugated with lysine to obtain the lysine conjugate:

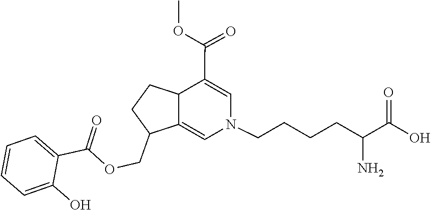 [SMILES: COC(=O)C1=CN(CCCCC(N)C(=O)O)C=C2C(COC(=O)c3ccccc3O)CCC12]

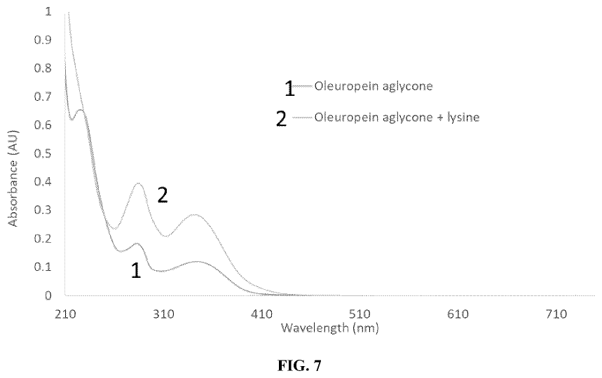

As shown in FIG. 6, reacting the compound of Example 7 (i.e. a hydrogenated genipin salicylate, called “genipin salicylate” in FIG. 6) with the amino acid lysine (called “amine” in FIG. 6) to mimic skin binding results in small shift in UV absorbance peak (no visible color is produced). Moreover, as also evident from the comparator “salicylic acid”, salicylic acid as such is a UV blocking agent and this activity is retained when attached to a hydrogenated genipin anchor molecule and when bound to a skin mimic.

The OL compound of example 8 was conjugated with lysine to obtain OL-lysine conjugate:

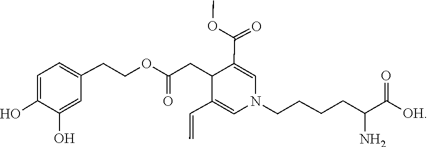 [SMILES: C=CC1=CN(CCCCC(N)C(=O)O)C=C(C(=O)OC)C1CC(=O)OCCc1ccc(O)c(O)c1]

As shown in FIG. 7, reacting the OL compound of example 8 with the amino acid to mimic skin binding results in small shift in UV absorbance peak and almost no visible color is produced.

### Example 10—Sunscreen Application

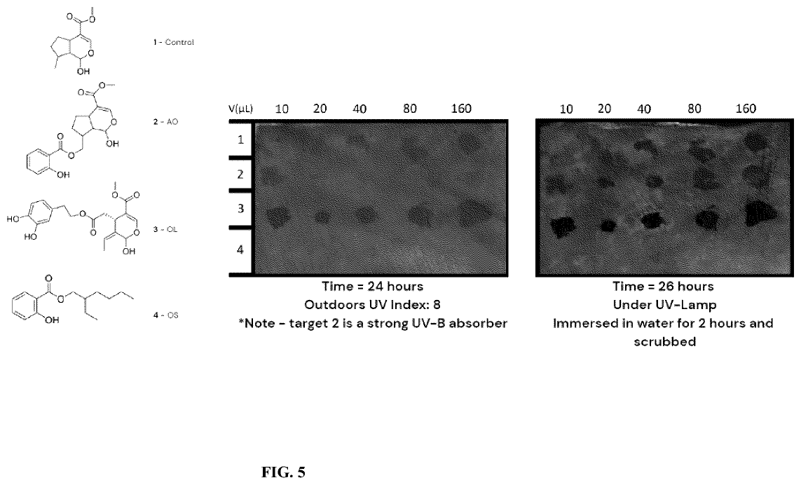

Compound 1 (1-control), compound of example 5 (2-AO), compound of example 6 (3-OL), and the conventional sunscreen agent octisalate (4-OS) as comparator were simultaneously tested. Briefly, 50 mM solutions of the compounds were made and various volumes were dropped onto the skin substrate (a porcine skin explant) to mimic application of the sunscreen. 2 hours after application a photo was taken (FIG. 4, left-hand side). Two hours later (i.e. 4 hours total), the skin was wiped with a wet tissue and another photo was taken (FIG. 4, right hand side). These photographs were taken under UV-light exposure (254 nm) with a normal camera. The glowing on the right-hand photograph of FIG. 4 indicates that the compounds according to the present disclosure are absorbing the light, even after the substrate was wiped with water whereas the non-binding sunscreen octisalate (OS) was easily removable from the skin.

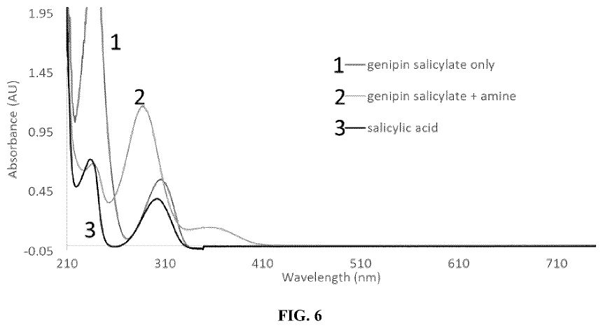

In a continuation of the first experiment, the skin sample was allowed to sit overnight, and another photo was taken outdoors in sunlight (UV index of 8) at 24 hours total contact time with a UV camera (equipped with a UV-B filter). The sample was then brought indoors, where it was submerged in water for 2 hours, and then scrubbed vigorously with a tissue. Then at 26 hours total contact time another photo was taken, this time under UV-lamp exposure (254 nm) with a UV camera (with a UV-B filter). The results are shown in FIG. 5. Dark color indicates the sunscreen is absorbing UV-light, even after the substrate was immersed in water for 2 hours and vigorously scrubbed. The non-binding sunscreen octisalate (OS) is no longer present.

### OTHER EMBODIMENTS

It is to be understood that while the present application has been described in conjunction with the detailed description thereof, the description is intended to illustrate and not limit the scope of the present application, which is defined by the scope of the appended claims. Other aspects, advantages, and modifications are within the scope of the following claims.

The present disclosure also relates to the following embodiments which are supplementary to and freely combinable with the above specification:

1. A topical composition comprising a compound of formula (I),

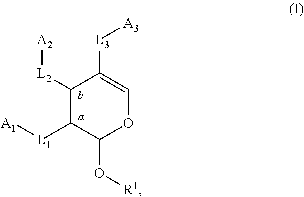

or a tautomer and/or a pharmaceutically acceptable salt thereof, wherein A₁, A₂ and A₃ independently from each other represent
a) a color-imparting moiety,
b) a UVA and/or UVB absorbing moiety, or
c) a C₁-C₃₀ moiety, H, hydroxyl, amino, or a halogen;
wherein R¹ represents hydrogen or a protective group hydrolysable under physiological conditions after application of the topical composition onto skin,
wherein L₁, L₂ and L₃ independently from each other represent a linker group or are absent, and
wherein L₁-A₁ and L₂-A₂ do not represent a moiety comprising an unsaturated C—C or C—N bond in alpha-position to the carbon atom marked “a” and “b”, respectively;
and an excipient suitable for topical administration.

2 The topical composition according to embodiment 1, wherein at least one of A₁, A₂ and A₃ is a moiety from above group a) or b).

3. The topical composition according to embodiment 1 or embodiment 2, wherein the color-imparting moiety has at least one absorption peak within the wavelength range of 380 to 790 nm.

4. The topical composition according to any preceding embodiment, wherein the UVA and/or UVB absorbing moiety has at least one absorption peak within the wavelength range of 280 to 379 nm.

5. The topical composition according to any preceding embodiment, wherein one or more of A₁, A₂ and A₃ represent a color-imparting moiety, wherein said moiety is:
a) an optionally substituted (hetero)aromatic moiety comprising, in combination with its optional substituents, 4 to 40 carbon atoms, or
b) an optionally substituted conjugated moiety comprising, in combination with its optional substituents, 6 to 40 carbon atoms and at least 3 conjugated C—C double bonds.

6. The topical composition according to any preceding embodiment, wherein one or more of A₁, A₂ and A₃ represents a UVA and/or UVB absorbing moiety, wherein said moiety is:
a) an optionally substituted (hetero)aromatic moiety comprising, in combination with its optional substituents, 4 to 36 carbon atoms, or
b) an optionally substituted conjugated moiety comprising, in combination with its optional substituents, 4 to 36 carbon atoms, and at least 2 conjugated C—C double bonds.

7. The topical composition according to any preceding embodiment, wherein one or more of A₁, A₂ and A₃ comprises a chromophore (dye moiety) selected from an azo group; a diazo group; a diphenylamine group; a nitroarylamine group; an azine group; an oxazine group; an acridine group; an indoline group; a sulfur dye group, in particular a thiazine group, a thiazole group, a thiazone group, a thianthrene group, or a phenothiazonethioanthrone group; a quinoid or quinone group; an anthraquinoid or anthraquinone group; a xanthene group; a naphthostyryl group; a diaryl methyl or triarylmethyl group; a benzodifuranone-based group; a formazan group; a phthalocyanine group; or a metal complex.

8. The topical composition according to any preceding embodiment, wherein one or more of A₁, A₂ and A₃ comprises a chromophore (dye moiety) selected from the following group of moieties:

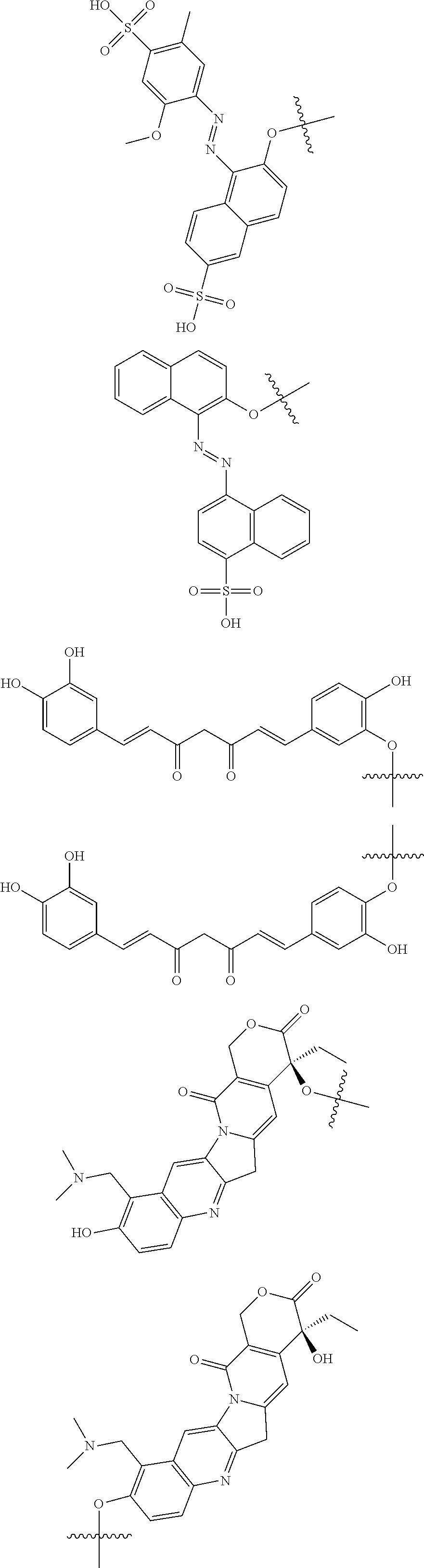

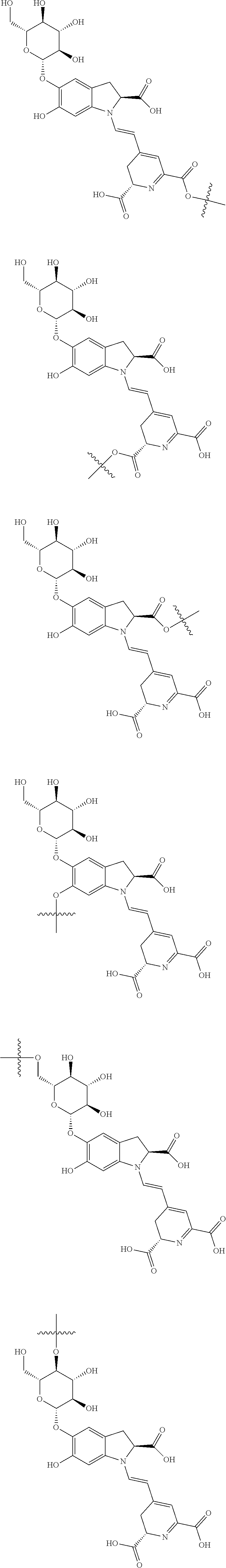

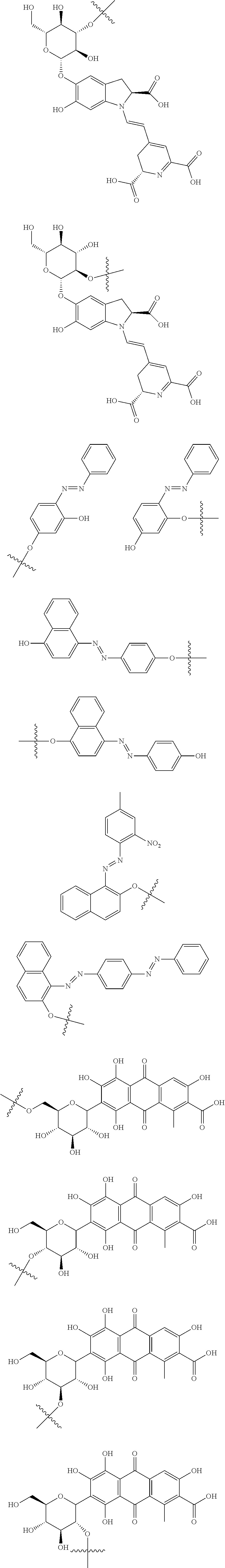

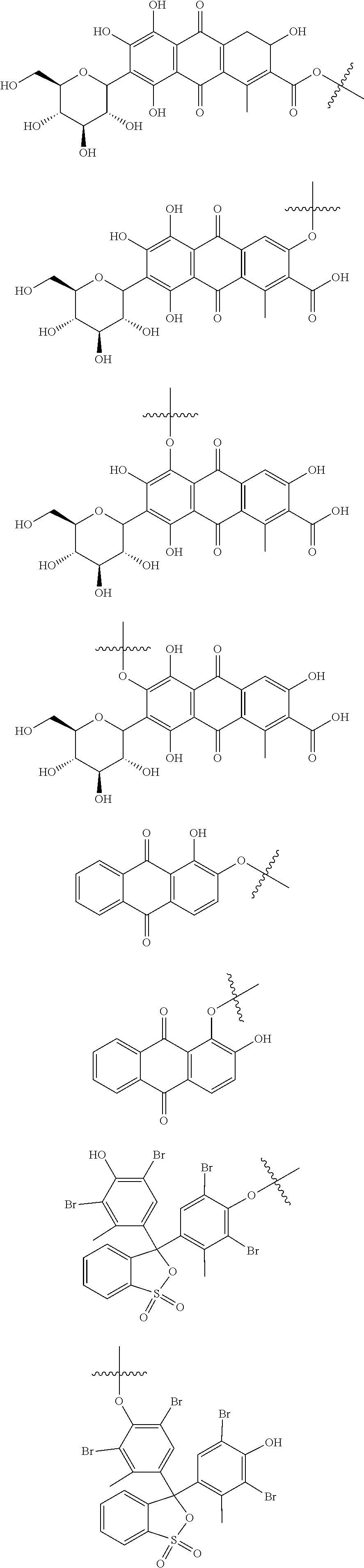

9. The topical composition according to any preceding embodiment, wherein one or more of A₁, A₂ and A₃ comprises a UVA and/or UVB absorbing moiety and being selected/derived from benzophenone group, a benzotriazole group, a benzone group, salicylic acid or a salicylic acid derivative, a benzocaine group, an esculin or an esculin derivative, a ferulic acid or a ferulic acid derivative, octinoxate or an octinoxate derivative, or octocrylene or an octocrylene derivative.

10. The topical composition according to any preceding embodiment, wherein one or more of A₁, A₂ and A₃ represents a UVA and/or UVB absorbing moiety, wherein said moiety is selected from the following group of moieties:

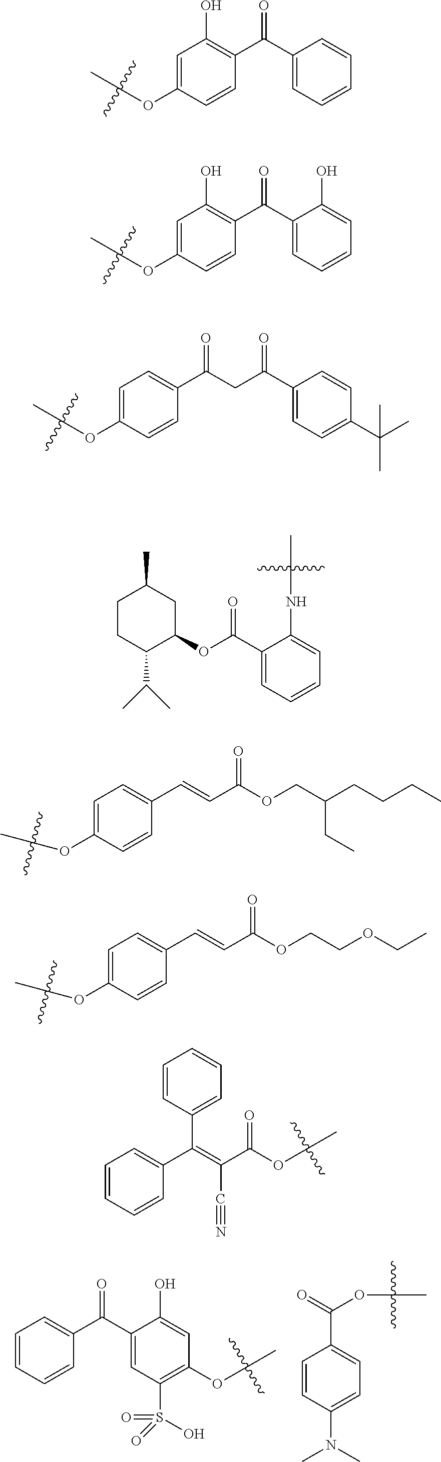

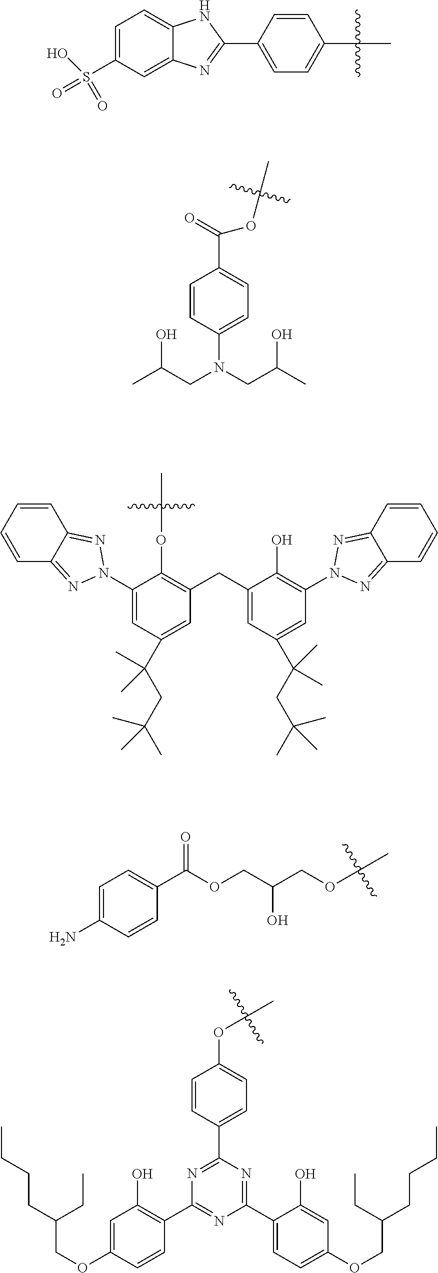 [SMILES: *c1ccc(-c2nc3cc(S(=O)(=O)O)ccc3[nH]2)cc1 ; *OC(=O)c1ccc(N(CC(C)O)CC(C)O)cc1 ; *Oc1c(Cc2cc(C(C)(C)CC(C)(C)C)cc(-n3nc4ccccc4n3)c2O)cc(C(C)(C)CC(C)(C)C)cc1-n1nc2ccccc2n1 ; *OCC(O)COC(=O)c1ccc(N)cc1 ; *Oc1ccc(-c2nc(-c3ccc(OCC(CC)CCCC)cc3O)nc(-c3ccc(OCC(CC)CCCC)cc3O)n2)cc1]

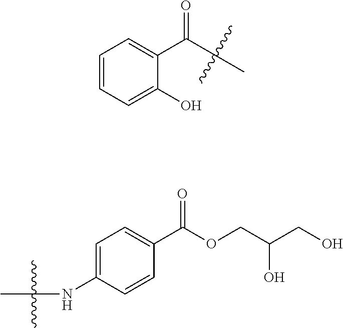 [SMILES: *C(=O)c1ccccc1O ; *Nc1ccc(C(=O)OCC(O)CO)cc1]

11. The topical composition according to any preceding embodiment, wherein L₁ and L₂, are present and independently from each other represent optionally substituted hydrocarbon moieties each comprising, in combination with their optional substituents, 1 to 8 carbon atoms; or wherein L₁ and L₂ form a 5-, 6-, 7- or 8-membered ring, in particular a cyclopentyl, a cyclohexyl, a pyrrolidinyl, a piperidinyl, a tetrahydrofuranyl or a tetrahydropyranyl.

12. The topical composition according to any preceding embodiment, wherein L₁ and L₂ form an optionally substituted 5- or 6-membered ring, in particular a cyclopentyl or cyclohexyl, to which A₁ and A₂ are attached, optionally via a group selected from: —O—, —S—, —C(O)—, —CO₂—, —O—C(O)—, —NH—C(O)—, —C(O)—NH—, —(CH₂)₁₋₄—, —(CH₂)₁₋₄—O— and —O—(CH₂)₁₋₄—.

13. The topical composition according to any preceding embodiment, wherein the C₁-C₃₀ moiety comprises 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms.

14. The topical composition according to any preceding embodiment, wherein the C₁-C₃₀ moiety is selected from a saturated or unsaturated, cyclic or acylic (hetero)alkyl comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms; and/or wherein the C₁-C₃₀ moiety is bound to L₁, L₂ and L₃, respectively, via a carbon atom, an oxygen atom, a nitrogen atom or a sulfur atom.

15. The topical composition according to any preceding embodiment, wherein A₃ represents a C₁-C₁₆ moiety selected from a saturated or unsaturated, cyclic or acylic (hetero)alkyl comprising 1 to 16, more specifically 1 to 12, and in particular 1 to 8, carbon atoms; 0 to 6, more specifically 0 to 4, and in particular 0 to 3 oxygen atoms; 0 to 6, more specifically 0 to 4, and in particular 0 to 3 nitrogen atoms; 0 to 6, more specifically 0 to 4, and in particular 0 to 3 sulfur atoms; and 0 to 6, more specifically 0 to 4, and in particular 0 to 3 halogen atoms.

16. The topical composition according to any preceding embodiment, wherein A₃ represents a C₁-C₁₆ moiety selected from a carboxylic acid or a salt thereof, a carboxylic acid ester; or a ketone.

17. The topical composition according to any preceding embodiment, wherein L₁ and L₂ are present and independently from each other represent optionally substituted hydrocarbon moieties each comprising, in combination with their optional substituents, 1 to 8 carbon atoms; or wherein L₁ and L₂ form a 5-, 6-, 7- or 8-membered ring, in particular a cyclopentyl, a cyclohexyl, a pyrrolidinyl, a piperidinyl, a tetrahydrofuranyl or a tetrahydropyranyl.

18. The topical composition according to any preceding embodiment, wherein L₁ and OR¹, together with the carbon atoms to which they are attached, form a 5- or 6-membered lactone.

19. The topical composition according to any preceding embodiment, wherein R₁ represents hydrogen or a C₁₋₆ acyl.

20. The topical composition according to any preceding embodiment, which is in the form of an aerosol spray, a cream, an emulsion, a solid, a liquid, a dispersion, a foam, an oil, a gel, a hydrogel, a lotion, a mousse, an ointment, a patch for transferring the composition onto skin, a substrate carrier comprising the topical composition, a pump spray comprising the topical composition, or a stick comprising the topical composition.

21. The topical composition according to any preceding embodiment, wherein the composition provides a sun protection factor (SPF) of at least 2 after application onto the skin.

22. The topical composition according to any preceding embodiment, wherein the excipient suitable for topical administration comprises one or more excipients selected from water, ethanol, isopropanol, n-propanol, ethylene glycol, diethylene glycol, a propylene glycol, and glycerol.

23. A compound of formula (VII) or the topical composition according to any one of embodiments 1 to 22 wherein the compound of formula (I) is a compound of formula (VII):

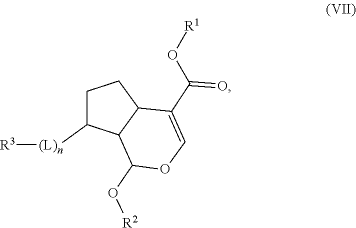

or a pharmaceutically acceptable salt thereof, wherein:
R¹ is selected from H and C₁₋₆ alkyl;
R² is selected from H and an acyl, in particular a C₁₋₆ acyl;
n is 0 or an integer selected from 1 to 10, wherein, if n=0, (L)ₙ represents a bond;
each L is independently selected from C(═O), N(R^{N}), O, (C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is a dye moiety. 24. The compound or composition of embodiment 23, wherein R² is selected from H, formyl, acetyl, propionyl, acrylyl, pivaloyl, and benzoyl.

25. The compound or composition of embodiment 24, wherein the (L)ₙ comprises at least one moiety selected from C₁₋₃ alkylene, C(═O), O, and NH.

26. The compound or composition of embodiment 24, wherein (L)ₙ comprises at least one CH₂O.

27. The compound or composition of embodiment 24, wherein the chain of (L)ₙ when n is larger than 2 comprises at least one C(═O)O.

28. The compound or composition of embodiment 24, wherein R³ comprises azo, diazo, anthraquinone, diphenylamine, nitroarylamine, coumaine, methane, naphthostyryl, quinophthalone, formazan, quinoline, indigo, indoline, or benzodifuranone-based dye.

29. The compound or composition of embodiment 28, wherein the dye comprises a functional group derivable by coupling a reactive functional group on the dye which is selected from a hydroxyl group (—OH), an amino group (—NH₂), a carboxylic acid group (—C(═O)OH), a sulfonic acid group (—S(═O)₂OH), and a phosphonate group (—P(═O)(OH)₂), to a precursor of the compound of Formula (VII).

30. The compound or composition of embodiment 23, wherein R³ is selected from any one of the following list of moieties:

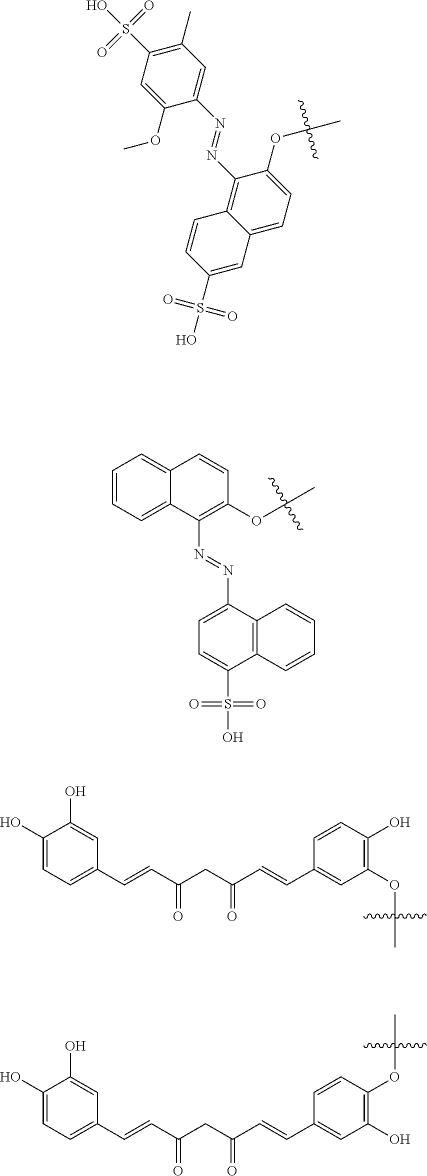 [SMILES: *Oc1ccc2cc(S(=O)(=O)O)ccc2c1/N=N/c1cc(C)c(S(=O)(=O)O)cc1OC ; *Oc1ccc2ccccc2c1/N=N/c1ccc(S(=O)(=O)O)c2ccccc12 ; *Oc1cc(/C=C/C(=O)CC(=O)/C=C/c2ccc(O)c(O)c2)ccc1O ; *Oc1ccc(/C=C/C(=O)CC(=O)/C=C/c2ccc(O)c(O)c2)cc1O]

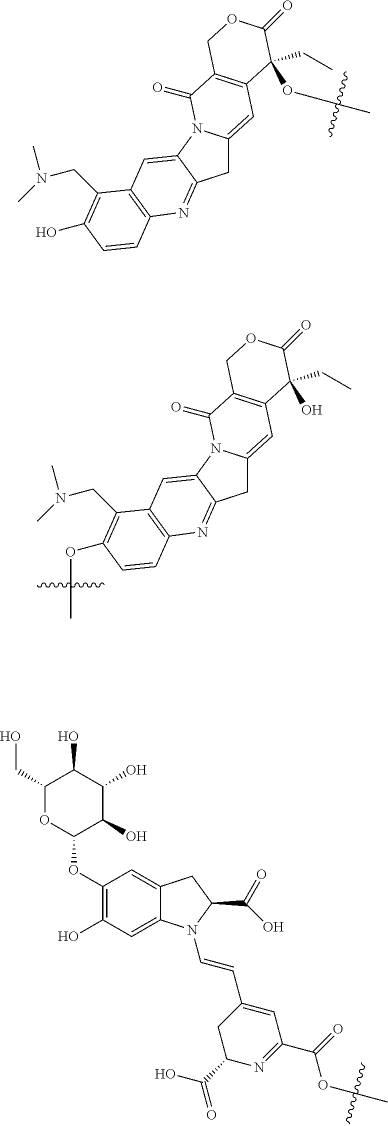 [SMILES: *O[C@]1(CC)C(=O)OCc2c1cc1n(c2=O)-c2cc3c(CN(C)C)c(O)ccc3nc2C1 ; *Oc1ccc2nc3c(cc2c1CN(C)C)-n1c(cc2c(c1=O)COC(=O)[C@]2(O)CC)C3 ; *OC(=O)C1=N[C@H](C(=O)O)CC(/C=C/N2c3cc(O)c(O[C@@H]4O[C@H](CO)[C@@H](O)[C@H](O)[C@H]4O)cc3C[C@H]2C(=O)O)=C1]

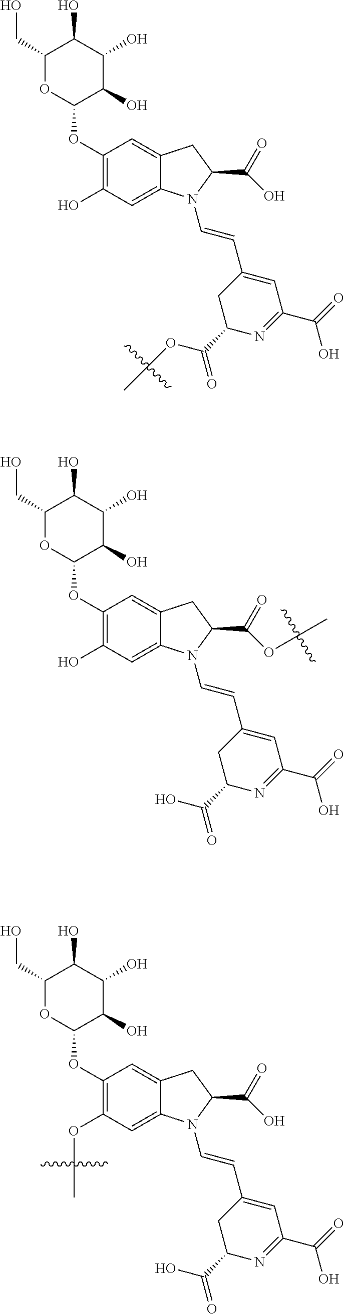 [SMILES: *OC(=O)[C@@H]1CC(/C=C/N2c3cc(O)c(O[C@@H]4O[C@H](CO)[C@@H](O)[C@H](O)[C@H]4O)cc3C[C@H]2C(=O)O)=CC(C(=O)O)=N1 ; *OC(=O)[C@@H]1Cc2cc(O[C@@H]3O[C@H](CO)[C@@H](O)[C@H](O)[C@H]3O)c(O)cc2N1/C=C/C1=CC(C(=O)O)=N[C@H](C(=O)O)C1 ; *Oc1cc2c(cc1O[C@@H]1O[C@H](CO)[C@@H](O)[C@H](O)[C@H]1O)C[C@@H](C(=O)O)N2/C=C/C1=CC(C(=O)O)=N[C@H](C(=O)O)C1]

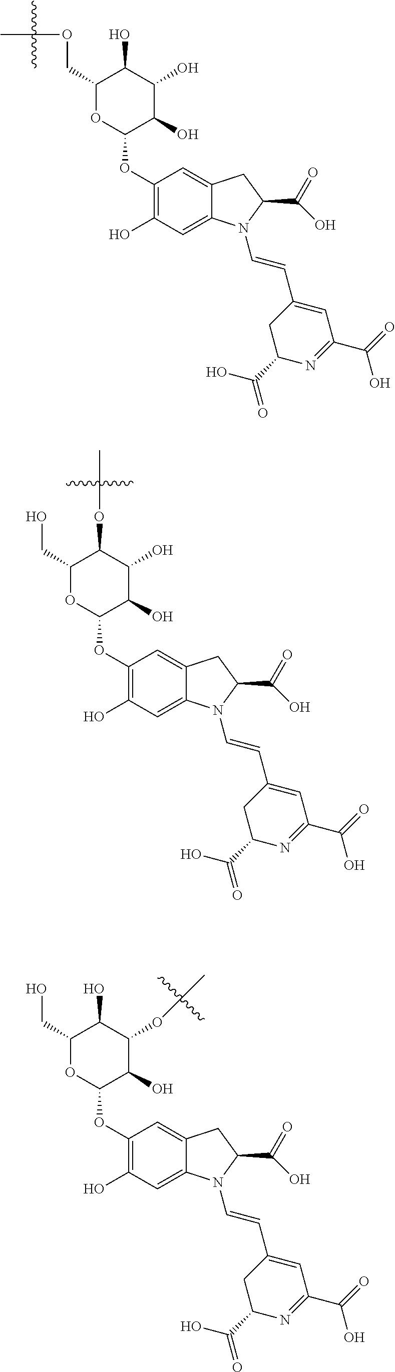 [SMILES: *OC[C@H]1O[C@@H](Oc2cc3c(cc2O)N(/C=C/C2=CC(C(=O)O)=N[C@H](C(=O)O)C2)[C@H](C(=O)O)C3)[C@H](O)[C@@H](O)[C@@H]1O ; *O[C@H]1[C@H](O)[C@@H](O)[C@H](Oc2cc3c(cc2O)N(/C=C/C2=CC(C(=O)O)=N[C@H](C(=O)O)C2)[C@H](C(=O)O)C3)O[C@@H]1CO ; *O[C@@H]1[C@@H](O)[C@H](Oc2cc3c(cc2O)N(/C=C/C2=CC(C(=O)O)=N[C@H](C(=O)O)C2)[C@H](C(=O)O)C3)O[C@H](CO)[C@H]1O]

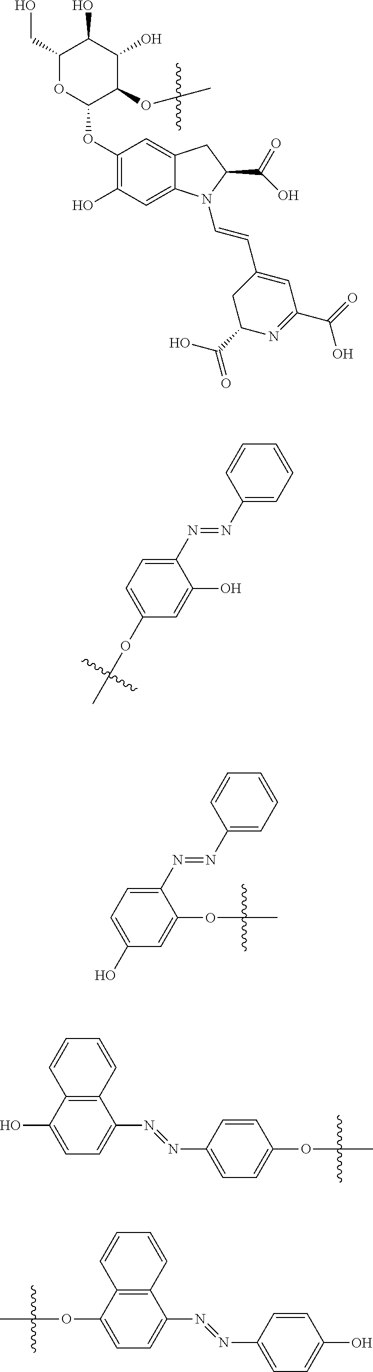 [SMILES: *O[C@H]1[C@H](Oc2cc3c(cc2O)N(/C=C/C2=CC(C(=O)O)=N[C@H](C(=O)O)C2)[C@H](C(=O)O)C3)O[C@H](CO)[C@@H](O)[C@@H]1O ; *Oc1ccc(/N=N/c2ccccc2)c(O)c1 ; *Oc1cc(O)ccc1/N=N/c1ccccc1 ; *Oc1ccc(/N=N/c2ccc(O)c3ccccc23)cc1 ; *Oc1ccc(/N=N/c2ccc(O)cc2)c2ccccc12]

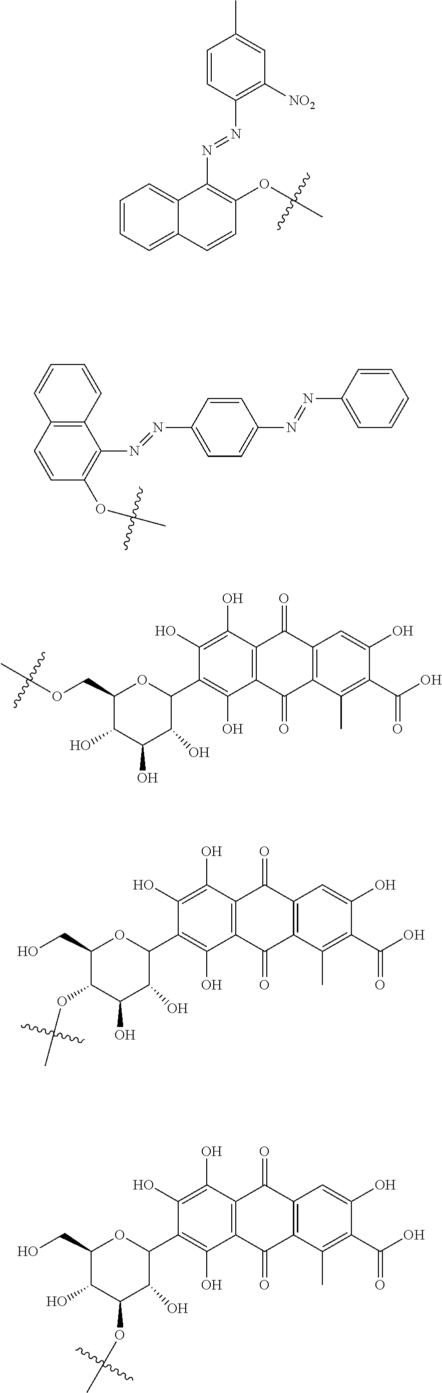 [SMILES: *Oc1ccc2ccccc2c1/N=N/c1ccc(C)cc1[N+](=O)[O-] ; *Oc1ccc2ccccc2c1/N=N/c1ccc(/N=N/c2ccccc2)cc1 ; *OC[C@H]1OC(c2c(O)c(O)c3c(c2O)C(=O)c2c(cc(O)c(C(=O)O)c2C)C3=O)[C@H](O)[C@@H](O)[C@@H]1O ; *O[C@H]1[C@H](O)[C@@H](O)C(c2c(O)c(O)c3c(c2O)C(=O)c2c(cc(O)c(C(=O)O)c2C)C3=O)O[C@@H]1CO ; *O[C@H]1[C@H](O)[C@@H](CO)OC(c2c(O)c(O)c3c(c2O)C(=O)c2c(cc(O)c(C(=O)O)c2C)C3=O)[C@@H]1O]

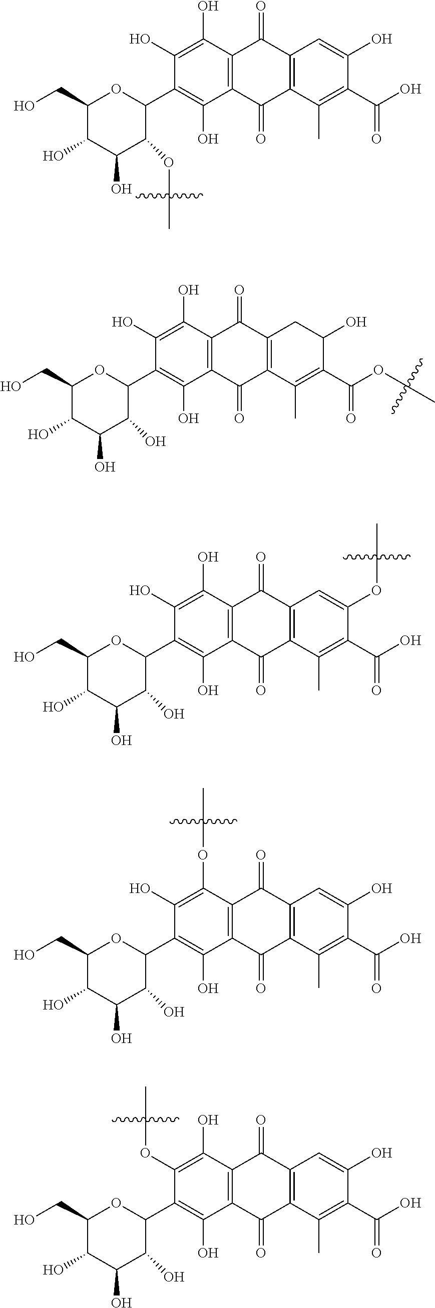 [SMILES: *O[C@H]1C(c2c(O)c(O)c3c(c2O)C(=O)c2c(cc(O)c(C(=O)O)c2C)C3=O)O[C@H](CO)[C@@H](O)[C@@H]1O ; *OC(=O)C1=C(C)C2=C(CC1O)C(=O)c1c(O)c(O)c(C3O[C@H](CO)[C@@H](O)[C@H](O)[C@H]3O)c(O)c1C2=O ; *Oc1cc2c(c(C)c1C(=O)O)C(=O)c1c(O)c(C3O[C@H](CO)[C@@H](O)[C@H](O)[C@H]3O)c(O)c(O)c1C2=O ; *Oc1c(O)c(C2O[C@H](CO)[C@@H](O)[C@H](O)[C@H]2O)c(O)c2c1C(=O)c1cc(O)c(C(=O)O)c(C)c1C2=O ; *Oc1c(O)c2c(c(O)c1C1O[C@H](CO)[C@@H](O)[C@H](O)[C@H]1O)C(=O)c1c(cc(O)c(C(=O)O)c1C)C2=O]

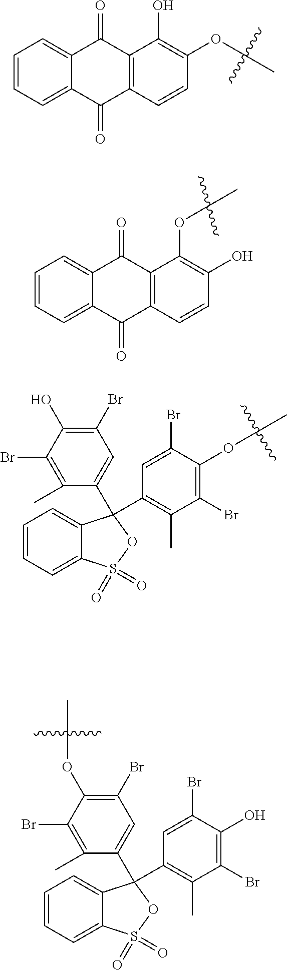

31. The compound or composition of embodiment 23, wherein the compound of Formula (VII) is selected from any one of the following formulae:

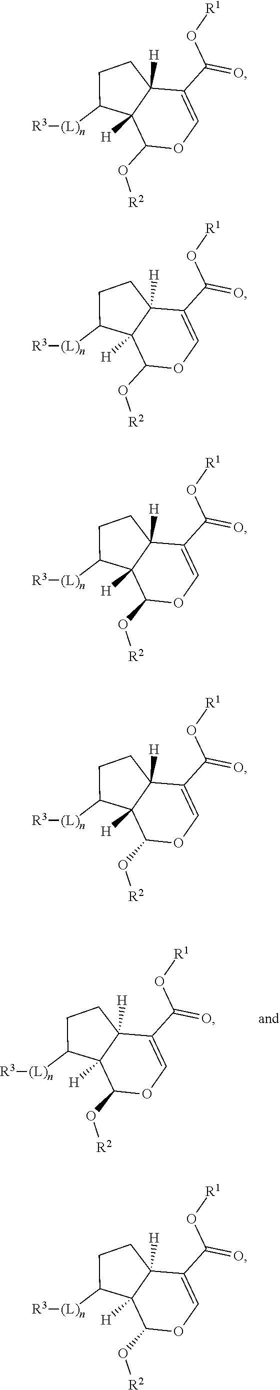

wherein R₁, R₂ and R₃ are defined as above in any preceding embodiment for formula (VII), and in particular as defined in embodiments 23 to 29, or a pharmaceutically acceptable salt thereof.

32. The compound or composition of embodiment 23, wherein the compound of Formula (VII) is selected from any one of the following compounds:

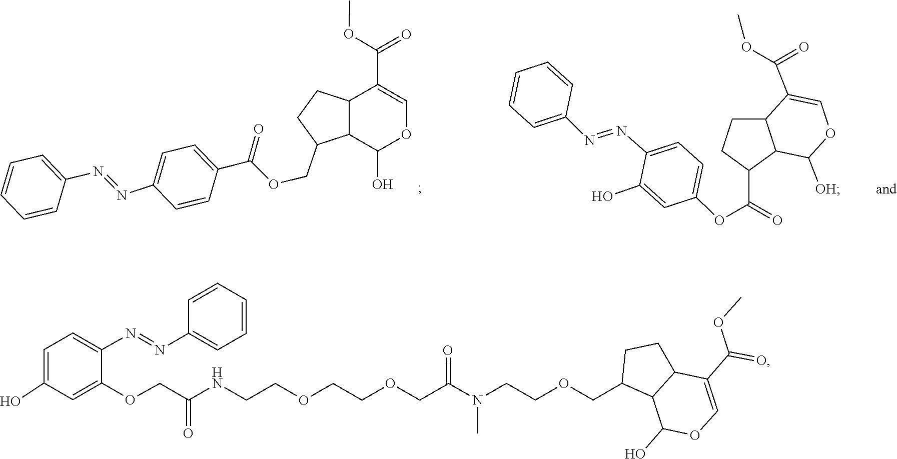 [SMILES: COC(=O)C1=COC(O)C2C(COC(=O)c3ccc(/N=N/c4ccccc4)cc3)CCC12 ; ";" ; COC(=O)C1=COC(O)C2C(C(=O)Oc3ccc(/N=N/c4ccccc4)c(O)c3)CCC12 ; "and" ; COC(=O)C1=COC(O)C2C(COCCN(C)C(=O)COCCOCCNC(=O)COc3cc(O)ccc3/N=N/c3ccccc3)CCC12]

or a pharmaceutically acceptable salt thereof.

33. A compound or composition of Formula (VIII) or the topical composition according to any preceding embodiments 1 to 22, wherein the compound of formula (I) is a compound of formula (VIII):

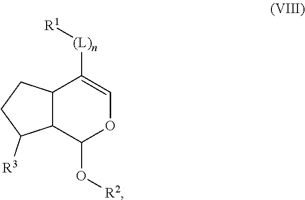

or a pharmaceutically acceptable salt thereof, wherein:
R¹ is a dye moiety, in particular a dye moiety as defined in embodiment 3;
R² is selected from H and an acyl, in particular a C₁₋₆ acyl; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

34. The compound or composition of embodiment 33, wherein R² is selected from H, formyl, acetyl, propionyl, acrylyl, pivaloyl, and benzoyl.

35. The compound or composition of embodiment 33, wherein the (L)ₙ comprises at least one moiety selected from C₁₋₃ alkylene, C(═O), O, and NH.

36. The compound or composition of embodiment 33, wherein (L)ₙ comprises at least one CH₂O.

37. The compound or composition of embodiment 33, wherein the chain of (L)ₙ when n is larger than 2 comprises at least one C(═O)O.

38. The compound or composition of embodiment 33, wherein R³ is selected from C₁₋₆ alkyl, C₂₋₆ alkenylene, C₂₋₆ alkynylene, C(O)OR^{a2}, and C(O)R^{b1}, wherein said C₁₋₆ alkyl is optionally substituted with OR^{a1} or NR^{c1}R^{d1}.

39. The compound or composition of embodiment 38, wherein R³ is selected from C₁₋₆ alkyl, C(O)OR^{a2}, C(O)R^{b1}, C₁₋₆ alkylene-OR^{a1}, and C₁₋₆ alkylene-NR^{c1}R^{d1}.

40. The compound or composition of any one of embodiments 33 to 39, wherein R^{a1}, R^{a2}, R^{b1}, R^{c1} and R^{d1} are each independently selected from is H or C₁₋₆ alkyl.

41. The compound or composition of embodiment 33, wherein R¹ comprises azo, diazo, anthraquinone, diphenylamine, nitroarylamine, coumarin, tri(C₆₋₁₀ aryl)methane, naphthostyryl, quinophthalone, formazan, quinoline, indigo, indoline, or benzodifuranone-based dye.

42. The compound or composition of embodiment 41, wherein the dye comprises a functional group derivable by coupling a reactive functional group on the dye which is selected from a hydroxyl group (—OH), an amino group (—NH₂), a carboxylic acid group (—C(═O)OH), a sulfonic acid group (—S(═O)₂OH), and a phosphonate group (—P(═O)(OH)₂), to a precursor of the compound of Formula (VII).

43. The compound or composition of embodiment 33, wherein R¹ is selected from any one of moieties recited in embodiment 30 for R³ of Formula (VII).

44. The compound or composition of embodiment 33, wherein the compound of Formula (VIII) is selected from any one of the following formulae:

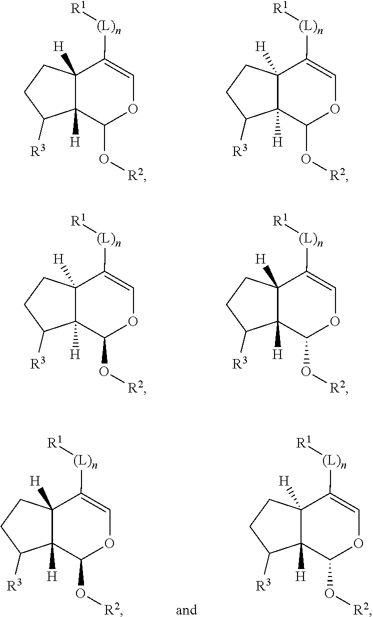

or a pharmaceutically acceptable salt thereof,
wherein R₁, R₂ and R₃ are defined as above in any preceding embodiment for formula (VIII), and in particular as defined in embodiments 23 to 29.

45. The compound or composition of embodiment 33, wherein the compound of formula (VIII) is selected from any one of the following compounds:

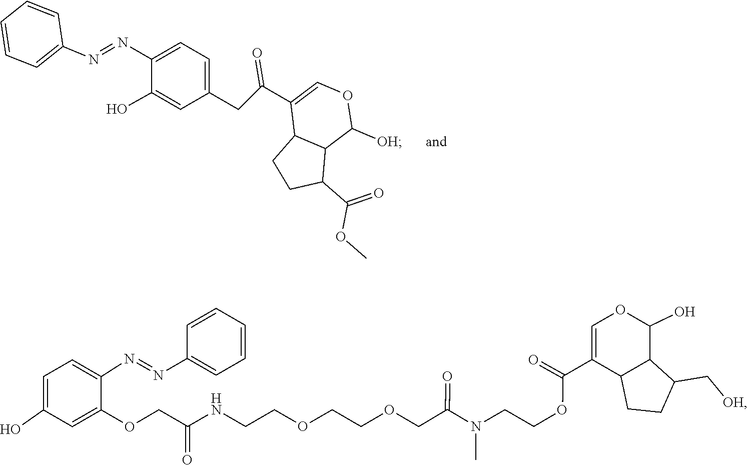 [SMILES: COC(=O)C1CCC2C(C(=O)Cc3ccc(/N=N/c4ccccc4)c(O)c3)=COC(O)C12 ; "and" ; CN(CCOC(=O)C1=COC(O)C2C(CO)CCC12)C(=O)COCCOCCNC(=O)COc1cc(O)ccc1/N=N/c1ccccc1]

or a pharmaceutically acceptable salt thereof.

46. A compound of Formula (VIIa) or the topical composition according to any one of embodiments 1 to 22 wherein the compound of formula (I) is a compound of formula (VIIa):

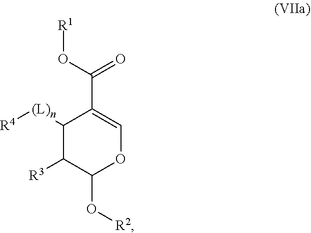

or a pharmaceutically acceptable salt thereof, wherein
R¹ is selected from H and C₁₋₆ alkyl;
R² is selected from H and an acyl; or
n is an integer selected from 1 to 10;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl;
R³ is selected from C₁₋₆ alkyl, C₂₋₆ alkenylene, C₂₋₆ alkynylene, C(O)R^{b1}, C(O)NR^{c1}R^{d1}, and C(O)OR^{a1};
R^{a1}, R^{b1}, R^{c1} and R^{d1} are each independently selected from H and C₁₋₆ alkyl; and in particular wherein R¹, R², R³, L, and n are as recited in embodiments 78-81; and
R⁴ is a dye moiety as recited for R³ in any one of embodiments 28-30.

47. A compound of Formula (VIIIa) or the topical composition according to any preceding embodiments 1 to 22 wherein the compound of formula (I) is a compound of formula (VIIIa):

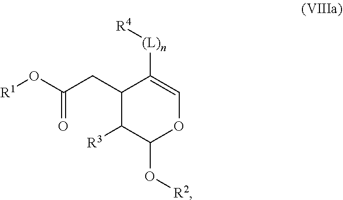

or a pharmaceutically acceptable salt thereof, wherein
R¹ is selected from H and C₁₋₆ alkyl;
R² is selected from H and an acyl, in particular a C₁-C₆ acyl; or
n is an integer selected from 1 to 10;
each L is independently selected from C(═O), N(R^{N}), O, (1-3 alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl;
R³ is selected from C₁₋₆ alkyl, C₂₋₆ alkenylene, C₂₋₆ alkynylene, C(O)R^{b1}, C(O)NR^{c1}R^{d1}, and C(O)OR^{a1};
R^{a1}, R^{b1}, R^{c1} and R^{d1} are each independently selected from H and C₁₋₆ alkyl; and
in particular wherein R¹, R², R³, L, and n are as recited in embodiments 88-91; and
R⁴ is a dye moiety as recited for R³ in any one of embodiments 28-30.

48. A composition comprising a compound of any one of embodiments 23-47, or a pharmaceutically acceptable salt thereof, and a solvent.

49. The composition of any preceding embodiment, in particular embodiment 48, further comprising a semi-volatile solubilizer.

50. The composition of any preceding embodiment, in particular embodiment 48, further comprising a film-forming agent.

51. The composition of any preceding embodiment, in particular embodiment 48, further comprising a thickening agent.

52. The composition of any preceding embodiment, in particular embodiment 48, wherein the solvent comprises water, methanol, ethanol, isopropanol, or a combination of two or more thereof.

53. A non-therapeutic method of applying color to a skin of a subject, the method comprising contacting the skin of the subject with an effective amount of a compound of any one of embodiments 23, 33, 46, or 47, or a pharmaceutically acceptable salt thereof, or of a composition of embodiment 48.

54. A compound of Formula (IX) or the topical composition according to any one of embodiments 1 to 22 wherein the compound of formula (I) is a compound of formula (IX):

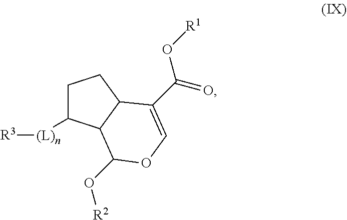

or a pharmaceutically acceptable salt thereof, wherein:
R¹ is selected from H and C₁₋₆ alkyl;
R² is selected from H and an acyl;
n is 0 or an integer selected from 1 to 10, wherein, if n=0, (L)ₙ represents a bond;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is a UV-absorbing moiety.

55. The compound or composition of embodiment 54, wherein R² is selected from H, formyl, acetyl, propionyl, acrylyl, pivaloyl, and benzoyl.

56. The compound or composition of embodiment 54, wherein the (L)ₙ comprises at least one moiety selected from C₁₋₃ alkylene, C(═O), O, and NH.

57. The compound or composition of embodiment 54, wherein (L)ₙ comprises at least one CH₂O.

58. The compound or composition of embodiment 54, wherein the chain of (L)ₙ when n is larger than 2 comprises at least one C(═O)O.

59. The compound or composition of embodiment 54, wherein R³ comprises phenyl, phenol, benzophenone, styrene, aminophenyl, aminophenol, indole, bendamidazole, benzotriazol, or triazine-based UV absorbing moiety.

60. The compound or composition of embodiment 59, wherein the UV absorbing moiety R³ comprises a functional group derivable by coupling a reactive functional group on the UV absorbing moiety which is selected from a hydroxyl group (—OH), an amino group (—NH₂), a carboxylic acid group (—C(═O)OH), a sulfonic acid group (—S(═O)₂OH), and a phosphonate group (—P(═O)(OH)₂), to a precursor of the compound of Formula (IX).

61. The compound or composition of embodiment 54, wherein R³ is selected from any one of the following moieties:

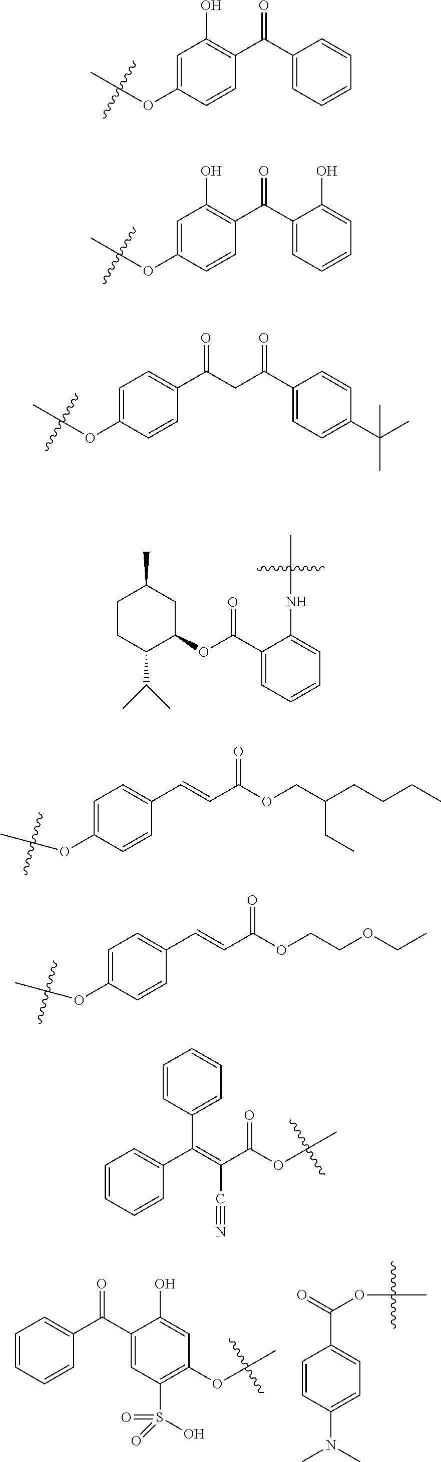

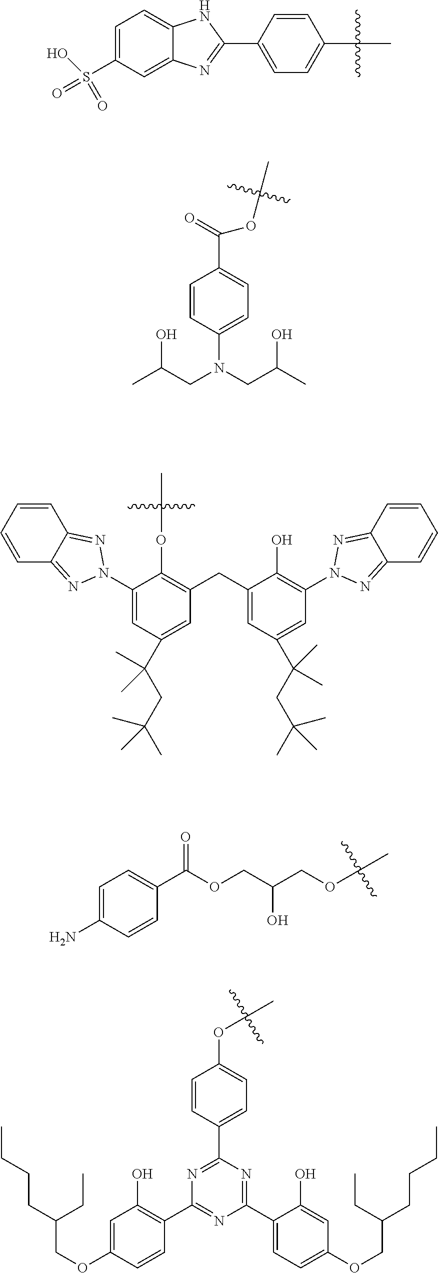 [SMILES: *c1ccc(-c2nc3cc(S(=O)(=O)O)ccc3[nH]2)cc1 ; *OC(=O)c1ccc(N(CC(C)O)CC(C)O)cc1 ; *Oc1c(Cc2cc(C(C)(C)CC(C)(C)C)cc(-n3nc4ccccc4n3)c2O)cc(C(C)(C)CC(C)(C)C)cc1-n1nc2ccccc2n1 ; *OCC(O)COC(=O)c1ccc(N)cc1 ; *Oc1ccc(-c2nc(-c3ccc(OCC(CC)CCCC)cc3O)nc(-c3ccc(OCC(CC)CCCC)cc3O)n2)cc1]

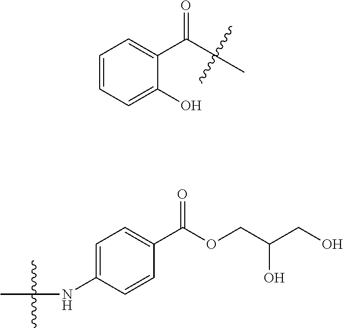 [SMILES: *C(=O)c1ccccc1O ; *Nc1ccc(C(=O)OCC(O)CO)cc1]

62. The compound or composition of embodiment 54, wherein the compound of Formula (IX) is selected from any one of the following formulae:

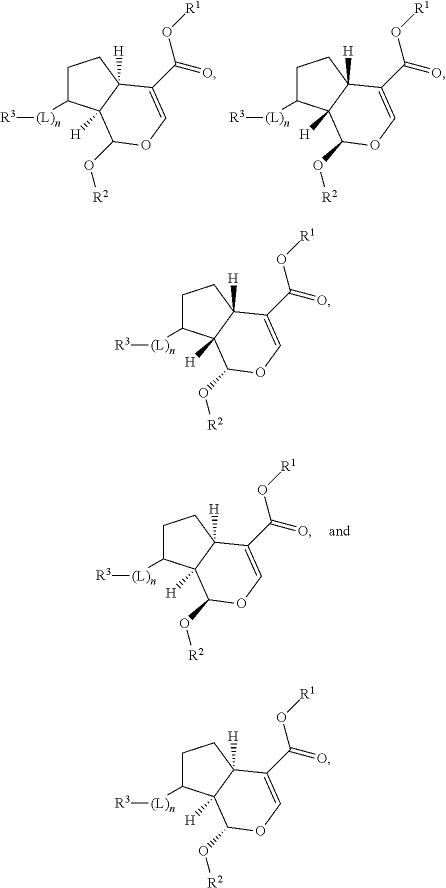

or a pharmaceutically acceptable salt thereof.

63. The compound or composition of embodiment 54, wherein the compound of Formula (IX) is selected from any one of the following compounds:

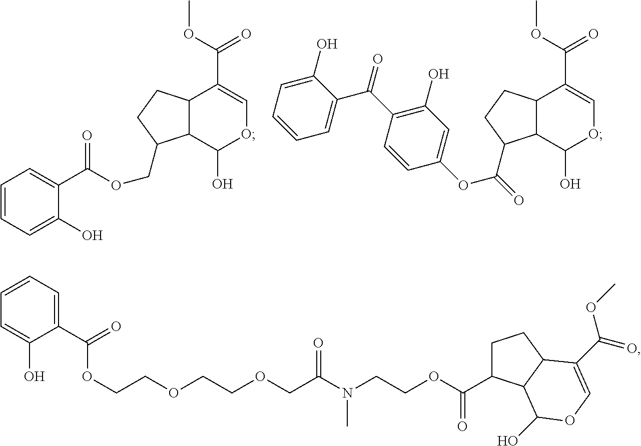

or a pharmaceutically acceptable salt thereof.

64. A compound of Formula (X) or the topical composition according to any one of embodiments 1 to 22 wherein the compound of formula (I) is a compound of formula (X):

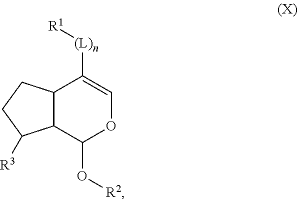

or a pharmaceutically acceptable salt thereof, wherein:
R¹ is a UV-absorbing moiety, in particular a UVA and/or UVB absorbing moiety as defined in embodiments 4, 6, 9 and 10;
R² is selected from H and an acyl, in particular C₁₋₆ acyl; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁-6 alkyl, C₁₋₄ haloalkyl, C₂₋₆ alkenyl, C₂₋₆ alkynyl, and a hydroxyl-protecting group; each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

65. The compound or composition of embodiment 64, wherein R² is selected from H, formyl, acetyl, propionyl, acrylyl, pivaloyl, and benzoyl.

66. The compound or composition of embodiment 64, wherein the (L)ₙ comprises at least one moiety selected from C₁₋₃ alkylene, C(═O), O, and NH.

67. The compound or composition of embodiment 64, wherein (L)ₙ comprises at least one CH₂O.

68. The compound or composition of embodiment 64, wherein the chain of (L)ₙ when n is larger than 2 comprises at least one C(═O)O.

69. The compound or composition of embodiment 64, wherein R³ is selected from C₁₋₆ alkyl, C₂₋₆ alkenylene, C₂₋₆ alkynylene, C(O)OR^{a2}, and C(O)R^{b1}, wherein said C₁₋₆ alkyl is optionally substituted with OR^{a1} or NR^{c1}R^{d1}.

70. The compound or composition of embodiment 69, wherein R³ is selected from C₁₋₆ alkyl, C(O)OR^{a2}, C(O)R^{b1}, C₁₋₆ alkylene-OR^{a1}, and C₁₋₆ alkylene-NR^{c1}R^{d1}.

71. The compound or composition of embodiment 64, wherein R^{a1}, R^{a2}, R^{b1}, R^{c1} and R^{d1} are each independently selected from H and C₁₋₆ alkyl.

72. The compound or composition of embodiment 64, wherein R¹ comprises phenyl, phenol, benzophenone, styrene, aminophenyl, aminophenol, indole, bendamidazole, benzotriazol, or triazine-based UV absorbing moiety.

73. The compound or composition of embodiment 72, wherein the UV absorbing moiety R¹ comprises a functional group derivable by coupling a reactive functional group on the UV absorbing moiety which is selected from a hydroxyl group (—OH), an amino group (—NH₂), a carboxylic acid group (—C(═O)OH), a sulfonic acid group (—S(═O)₂OH), and a phosphonate group (—P(═O)(OH)₂), to a precursor of the compound of Formula (X).

74. The compound or composition of embodiment 73, wherein R¹ is selected from any one of moieties recited in embodiment 61 for R³ of Formula (IX).

75. The compound or composition of embodiment 74, wherein the compound of Formula (X) is selected from any one of the following formulae:

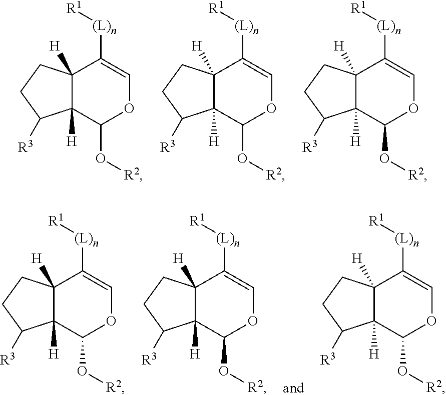

or a pharmaceutically acceptable salt thereof, wherein R₁, R₂ and R₃ are defined as in embodiment 64, and in particular as defined in any of embodiments 65 to 72.

76. The compound or composition of embodiment 64, wherein the compound of Formula (X) is selected from any one of the following compounds:

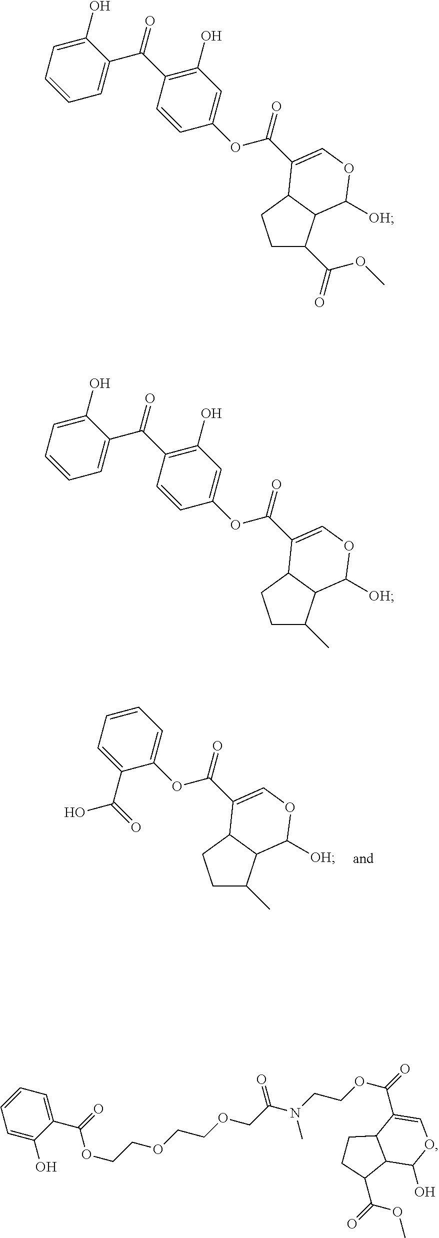 [SMILES: COC(=O)C1CCC2C(C(=O)Oc3ccc(C(=O)c4ccccc4O)c(O)c3)=COC(O)C12 ; CC1CCC2C(C(=O)Oc3ccc(C(=O)c4ccccc4O)c(O)c3)=COC(O)C12 ; CC1CCC2C(C(=O)Oc3ccccc3C(=O)O)=COC(O)C12 ; COC(=O)C1CCC2C(C(=O)OCCN(C)C(=O)COCCOCCOC(=O)c3ccccc3O)=COC(O)C12]

or a pharmaceutically acceptable salt thereof.

77. A compound of Formula (XIII) or the topical composition according to any one of embodiments 1 to 22 wherein the compound of formula (I) is a compound of formula (XIII):

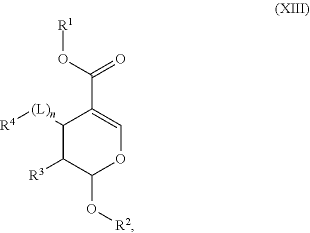

or a pharmaceutically acceptable salt thereof, wherein:
R¹ is selected from H and C₁₋₆ alkyl;
R² is selected from H and an acyl; or
n is 0 or an integer selected from 1 to 10, wherein, if n=0, (L)ₙ represents a bond;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl;
R⁴ is a UV-absorbing moiety, in particular a UVA and/or UVB absorbing moiety as defined in embodiments 4, 6, 9 and 10;
R³ is selected from C₁₋₆ alkyl, C₂₋₆ alkenylene, C₂₋₆ alkynylene, C(O)R^{b1}, C(O)NR^{c1}R^{d1}, and C(O)OR^{a1}; and
R^{a1}, R^{b1}, R^{c1} and R^{d1} are each independently selected from H and C₁₋₆ alkyl.

78. The compound or composition of embodiment 77, wherein R² is selected from H, formyl, acetyl, propionyl, acrylyl, pivaloyl, and benzoyl.

79. The compound or composition of embodiment 77, wherein the (L)ₙ comprises at least one moiety selected from C₁₋₃ alkylene, C(═O), O, and NH.

80. The compound or composition of embodiment 77, wherein (L)ₙ comprises at least one CH₂O.

81. The compound or composition of embodiment 77, wherein the chain of (L)ₙ when n is larger than 2 comprises at least one C(═O)O.

82. The compound or composition of embodiment 77, wherein R⁴ comprises phenyl, phenol, benzophenone, styrene, aminophenyl, aminophenol, indole, bendamidazole, benzotriazol, or triazine-based UV absorbing moiety.

83. The compound or composition of embodiment 77, wherein the UV absorbing moiety comprises a functional group derivable by coupling a reactive functional group on the UV absorbing moiety which is selected from a hydroxyl group (—OH), an amino group (—NH₂), a carboxylic acid group (—C(═O)OH), a sulfonic acid group (—S(═O)₂OH), and a phosphonate group (—P(═O)(OH)₂), to a precursor of the compound of Formula (XIII).

84. The compound or composition of embodiment 77, wherein R⁴ is any one of moieties as defined for R³ in embodiment 61.

85. The compound or composition of embodiment 77, wherein the compound of Formula (XIII) is selected from any one of the following formulae:

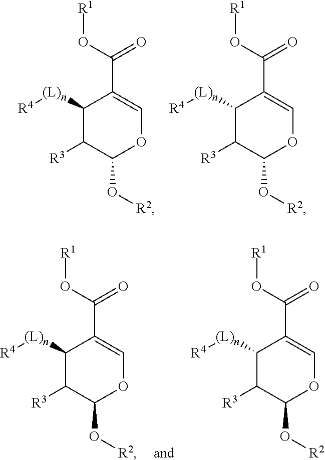

or a pharmaceutically acceptable salt thereof,
wherein R₁, R₂, R₃ and R₄ are defined as in embodiment 77, and in particular as defined in any of embodiments 78 to 84.

86. The compound or composition of embodiment 77, wherein the compound of Formula (XIII) is selected from any one of the following compounds:

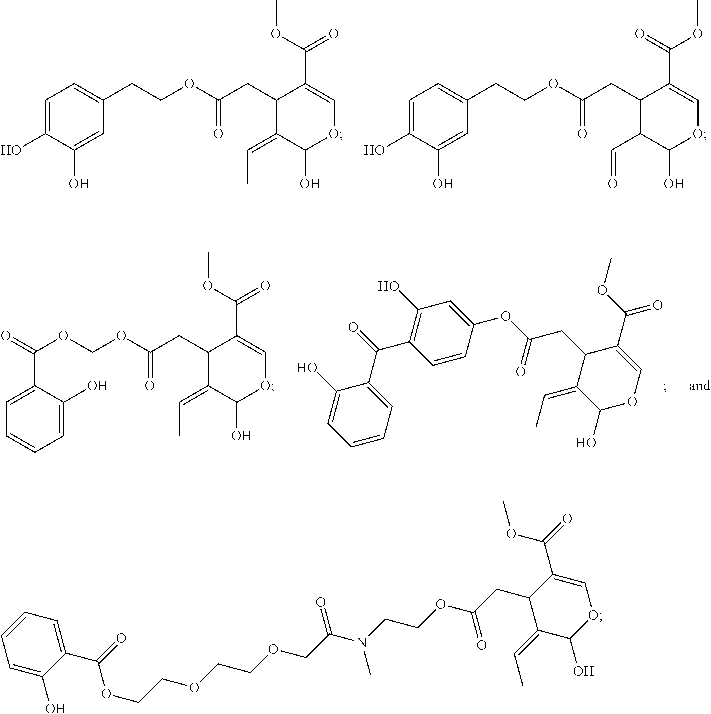 [SMILES: C/C=C1\C(O)OC=C(C(=O)OC)C1CC(=O)OCCc1ccc(O)c(O)c1 ; COC(=O)C1=COC(O)C(C=O)C1CC(=O)OCCc1ccc(O)c(O)c1 ; C/C=C1\C(O)OC=C(C(=O)OC)C1CC(=O)OCOC(=O)c1ccccc1O ; C/C=C1\C(O)OC=C(C(=O)OC)C1CC(=O)Oc1ccc(C(=O)c2ccccc2O)c(O)c1 ; C/C=C1\C(O)OC=C(C(=O)OC)C1CC(=O)OCCN(C)C(=O)COCCOCCOC(=O)c1ccccc1O]

or a pharmaceutically acceptable salt thereof.

87. A compound of Formula (XIV) or the topical composition according to any preceding embodiments 1 to 22 wherein the compound of formula (I) is a compound of formula (XIV):

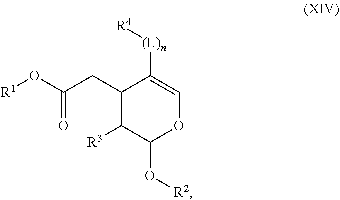

or a pharmaceutically acceptable salt thereof, wherein:
R¹ is selected from H and C₁₋₆ alkyl;
R² is selected from H and an acyl; or
n is 0 or an integer selected from 1 to 10, wherein, if n=0, (L)ₙ represents a bond;
each L is independently selected from C(═O), N(R^{N}), O, (C₁₋₃ alkylene-O—)ₓ, (—O—C₁₋₃ alkylene-)ₓ, and —C₁₋₃ alkylen wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl;
R⁴ is a UV-absorbing moiety, in particular a UVA and/or UVB absorbing moiety as defined in any one of embodiments 4, 6, 9 or 10;
R³ is selected from C₁₋₆ alkyl, C₂₋₆ alkenylene, C₂₋₆ alkynylene, C(O)R^{b1}, C(O)NR^{c1}R^{d1}, and C(O)ORₐ₁; and
R^{a1}, R^{b1}, R^{c1} and R^{d1} are each independently selected from H and C₁₋₆ alkyl.

88. The compound or composition of embodiment 87, wherein R² is selected from H, formyl, acetyl, propionyl, acrylyl, pivaloyl, and benzoyl.

89. The compound or composition of embodiment 87, wherein the (L)ₙ comprises at least one moiety selected from C₁₋₃ alkylene, C(═O), O, and NH.

90. The compound or composition of embodiment 87, wherein (L)ₙ comprises at least one CH₂O.

91. The compound or composition of embodiment 87, wherein the chain of (L)ₙ when n is larger than 2 comprises at least one C(═O)O.

92. The compound or composition of embodiment 87, wherein R⁴ comprises phenyl, phenol, benzophenone, styrene, aminophenyl, aminophenol, indole, bendamidazole, benzotriazol, or triazine-based UV absorbing moiety.

93. The compound or composition of embodiment 87, wherein the UV absorbing moiety comprises a functional group derivable by coupling a reactive functional group on the UV absorbing moiety which is selected from a hydroxyl group (—OH), an amino group (—NH₂), a carboxylic acid group (—C(═O)OH), a sulfonic acid group (—S(═O)₂OH), and a phosphonate group (—P(═O)(OH)₂), to a precursor of the compound of Formula (XIV).

94. The compound or composition of embodiment 77, wherein R⁴ is any one of moieties as defined for R³ in embodiment 61.

95. The compound or composition of embodiment 87, wherein the compound of Formula (XIV) is selected from any one of the following formulae:

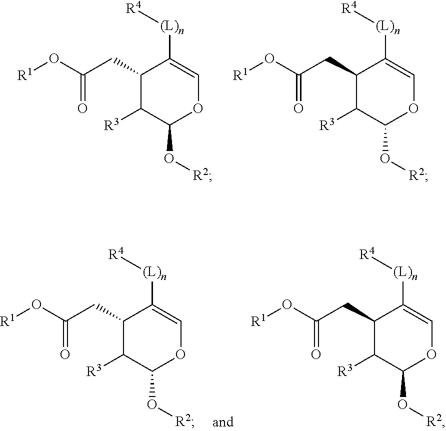

or a pharmaceutically acceptable salt thereof;
wherein R₁, R₂, R₃ and R₄ are defined as in embodiment 87, and in particular as defined in any of embodiments 88 to 94.

96. The compound or composition of embodiment 87, wherein the compound of formula (XIV) is selected from any one of the following compounds:

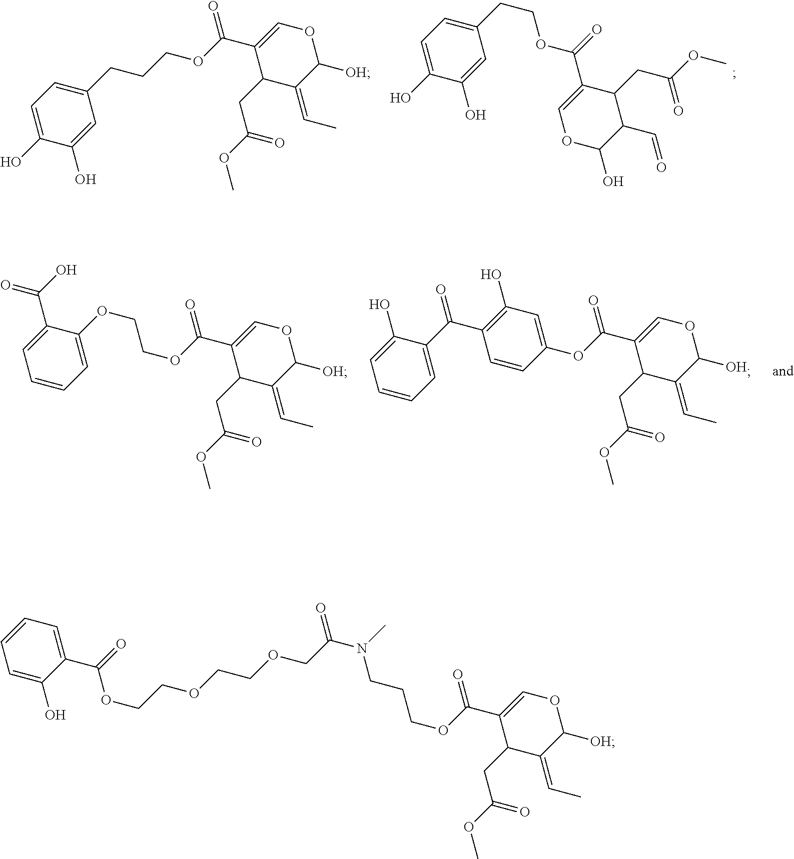

or a pharmaceutically acceptable salt thereof.

97. A topical sunscreen composition comprising a compound of any one of embodiments 54-96, or a pharmaceutically acceptable salt thereof, and an excipient suitable for topical administration.

98. The composition of embodiment 97, which is in the form of an aerosol spray, a cream, an emulsion, a solid, a liquid, a dispersion, a foam, an oil, a gel, a hydrogel, a lotion, a mousse, an ointment, a patch for transferring the composition onto skin, a substrate carrier comprising the topical composition, a pump spray comprising the topical composition, or a stick comprising the topical composition.

99. A compound of formula (II) or the topical composition according to any one of embodiments 1 to 22 wherein the compound of formula (I) is a compound of formula (II),

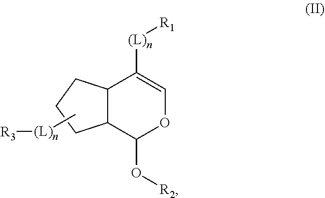

or a tautomer and/or a pharmaceutically acceptable salt thereof,
wherein:
R¹ and R³ independently from each other represent H; a C₁-C₃₀ moiety comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms; —SO₃H; —NO₂; —NH₂; —OH; —SH; or halogen, in particular Cl, Br or F; and, in case of R³, oxo;
R² is selected from H or a protective group hydrolysable under physiological conditions after application in a topical composition onto skin; and
optionally wherein (L)ₙR³ and OR², together with the carbon atoms to which they are attached, form a tetrahydrofuran ring which is optionally substituted with OR^{a1} or oxo;
n is 0 or is an integer selected from 1 to 10, wherein, if n=0, (L)ₙ represents a bond;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl;
wherein optionally the compound of formula (II) is not any one of the following compounds:

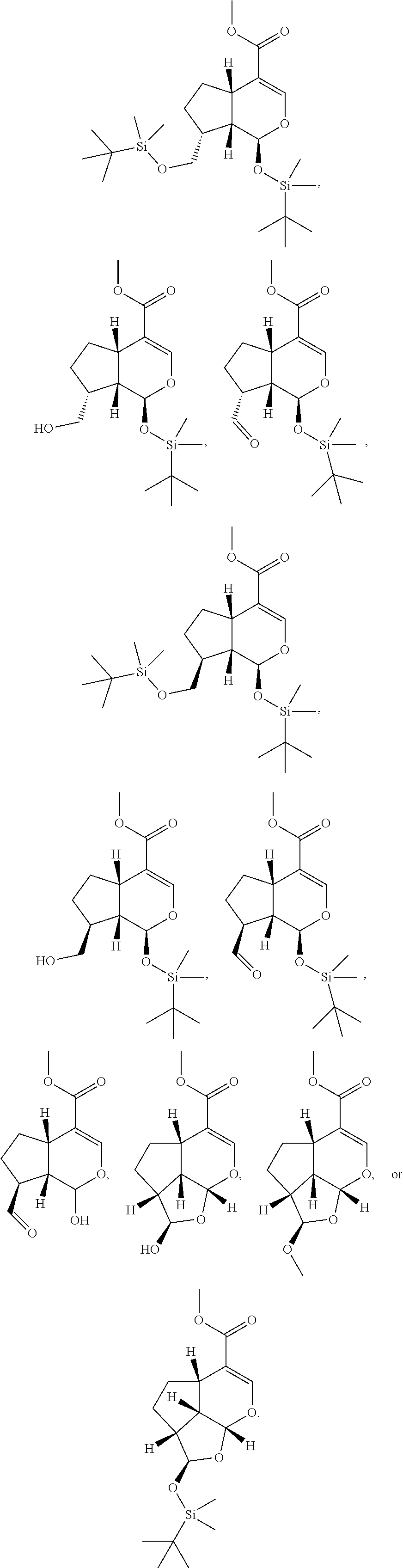

100. The compound or composition according to embodiment 99, wherein R¹ and/or R³ represent said C₁-C₃₀ moiety which further is a color-imparting moiety, in particular a color-imparting moiety having at least one absorption peak within the wavelength range of 380 to 790 nm.

101. The compound or composition according to embodiment 99 or embodiment 100, wherein R¹ and/or R³ represent said C₁-C₃₀ moiety which further is a UVA and/or UVB absorbing moiety, in particular a UVA and/or UVB absorbing moiety having at least one absorption peak within the wavelength range of 280 to 379 nm.

102. The compound or composition according to any one of embodiments 99 to 101, wherein said C₁-C₃₀ moiety of R¹ and/or R³ is selected from:
a) an optionally substituted saturated or unsaturated, cyclic or acyclic hydrocarbon comprising, together with its optional substituents, 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms;
b) an optionally substituted (hetero)aromatic moiety comprising, together with its optional substituents, 4 to 30, more specifically 4 to 16, and in particular 4 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms.

103. The compound or composition according to any one of embodiments 99 to 101, wherein the compound of formula (II) is a compound of formula (III),

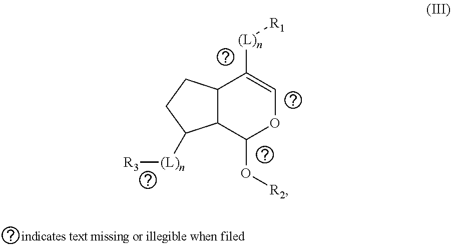

wherein R¹, R², R³, L and n are as defined in embodiment 99.

104. The compound or composition according to any one of embodiments 99 to 103, wherein the C₁-C₃₀ moiety of R³ is attached to (L)ₙ or the cyclopentyl ring of formulae (II) or (III) via a carbon atom, an oxygen atom, a nitrogen atom or a sulfur atom.

105. The compound or composition according to any one of embodiments 99 to 104, wherein the C₁-C₃₀ moiety of R¹ is attached to (L)ₙ or to the 3,4-dihydro-2H-pyran ring of formulae (II) or (III) via a carbon atom, an oxygen atom, a nitrogen atom or a sulfur atom.

106. The compound or composition according to any one of embodiments 99 to 105, wherein (L)ₙR³ represents R^{a}, O—R^{a}, SR^{a}, (CH₂)₁₋₄—O—R^{a}, C(O)R^{a}, CO₂R, O—C(O)R^{a}, NR^{a}₂, NHR^{a}, NHC(O)R^{a}, NR^{a}C(O)R^{a}, or oxo, wherein R^{a} independently from each other represents a C₁-C₁₈ moiety comprising 1 to 30, more specifically 1 to 16, and in particular 1 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms.

107. The compound or composition according to embodiment 106, wherein R^{a}, independently from each other, represents C₁₋₁₈ alkyl, C₂₋₁₈ alkenylene, C₂₋₁₈ alkynylene, C₆₋₁₈ aryl, C₄₋₁₈ heteroaryl comprising 1-4 nitrogen atoms, 1-3 oxygen atoms and/or 1-2 sulfur atoms, wherein any of the aforementioned groups is optionally further substituted with the proviso that the total sum of the elements recited for R^{a} in embodiment 106 is not exceeded.

108. The compound or composition according to embodiment 106 or embodiment 107, wherein (L)ₙR³ represents (CH₂)₁₋₄—O—R^{a}, O—R^{a}, C(O)R^{a}, CO₂R^{a}, or O—C(O)R^{a}, wherein R^{a} represents an optionally substituted phenyl.

109. The compound or composition according to any one of embodiments 106 to 108, wherein (L)ₙR¹ represents CO₂R^{a}, C(O)R^{a} or R^{a}, wherein R^{a} represents optionally substituted phenyl, in particular wherein the optionally substituted phenyl comprises 6 to 30, more specifically 6 to 16, and in particular 6 to 12, carbon atoms; 0 to 12, more specifically 0 to 8, and in particular 0 to 6 oxygen atoms; 0 to 8, more specifically 0 to 6, and in particular 0 to 4 nitrogen atoms; 0 to 6, more specifically 0 to 4, and in particular 0 to 3 sulfur atoms; and 0 to 10, more specifically 0 to 8, and in particular 0 to 6 halogen atoms.

110. The compound or composition according to any one of embodiments 106 to 109, wherein R^{a} is further substituted with one or more moieties selected from the group consisting of: —C₁₋₄ alkyl, —O—C₁₋₄ alkyl, —S—C₁₋₄ alkyl, —NH—C₁₋₄ alkyl, —N(C₁₋₄ alkyl)₂, —C(O)C₁₋₄ alkyl, —CO₂C₁₋₄ alkyl, —O₂C—C₁₋₄ alkyl, —C(O)NH—C₁₋₄ alkyl, —C(O)N(C₁₋₄ alkyl)₂, —NH—C(O)C₁₋₄ alkyl, —N(C₁₋₄ alkyl)-C(O)C₁₋₄ alkyl, —SO₃H, —NO₂, —NH₂, —OH, —SH, —COOH, —C(O)H, halogen, in particular Cl, Br or F; and —CF₃.

111. The compound or composition according to embodiment 99, wherein the compound of formula (II) is a compound of Formula (IV):

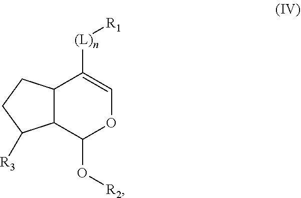

or a pharmaceutically acceptable salt thereof, wherein:
R¹ is selected from H and a carboxylic acid-protecting group;
R² is as defined in embodiment 99; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

112. The compound or composition according to embodiment 111, wherein R¹ is selected from H and C₁₋₆ alkyl.

113. The compound or composition according to embodiment 111 or embodiment 112, wherein R³ is selected from C₁₋₆ alkyl, C₂₋₆ alkenylene, C₂₋₆ alkynylene, C(O)OR^{a2}, and C(O)R^{b1}, wherein said C₁₋₆ alkyl is optionally substituted with OR^{a1} or NR^{c1}R^{d1}.

114. The compound or composition according to any one of embodiments 111 to 113, wherein R^{a1} is selected from H and a hydroxyl-protecting group.

115. The compound or composition according to any one of embodiments 111 to 114, wherein R^{a2} is selected from H and C₁₋₆ alkyl.

116. The compound or composition according to any one of embodiments 111 to 115, wherein R^{b1} is selected from H and C₁₋₆ alkyl.

117. The compound or composition according to any one of embodiments 111 to 116, wherein R^{c1} is H or C₁₋₆ alkyl.

118. The compound or composition according to any one of embodiments 111 to 117, wherein R^{d1} is an amino-protecting group.

119. The compound or composition according to any one of embodiments 111 to 118, wherein:
R¹ is selected from H and C₁₋₆ alkyl;
R² is selected from H and a hydroxyl-protecting group;
R³ is selected from C₁₋₆ alkyl, C₂₋₆ alkenylene, C₂₋₆ alkynylene, C(O)OR^{a2}, and C(O)R^{b1}, wherein said C₁₋₆ alkyl is optionally substituted with OR^{a1} orNR^{c1}R^{d1};
R^{a1} is selected from H and a hydroxyl-protecting group;
R^{a2} is selected from H, C₁₋₆ alkyl, and a carboxyl-protecting group;
R^{b1} is selected from H and C₁₋₆ alkyl;
R^{c1} is H or C₁₋₆ alkyl; and
R^{d1} is an amino-protecting group.

120. The compound or composition according to any one of embodiments 111 to 119, wherein, wherein:
R¹ is selected from H and C₁₋₆ alkyl;
R² is selected from H, methoxymethyl, benzyloxymethyl, benzyl, p-methoxybenzyl, tri(C₁₋₆ alkyl)silyl, and an acyl;
R³ is selected from C₁₋₆ alkyl, C(O)R^{b1}, C(O)OR^{a2}, C₁₋₆ alkylene-OR^{a1}, and C₁₋₆ alkylene-NR^{c1}R^{d1};
R^{a1} is selected from H, methoxymethyl, benzyloxymethyl, benzyl, p-methoxybenzyl, tri(C₁₋₆ alkyl)silyl, and an acyl;
R^{a2} is selected from H and C₁₋₆ alkyl;
R^{b1} is selected from H and C₁₋₆ alkyl;
R^{c1} is H or C₁₋₆ alkyl; and
R^{d1} is selected from 9-fluorenylmethoxycarbonyl (Fmoc), the tert-butyloxycarbonyl (Boc), benzyl, benzyl carbamate (Cbz), phthalimide, p-toluenesulfonamide (Ts), and triphenylmethylamine (Tr).

121. The compound or composition according to any one of embodiments 111 to 119, wherein R³ is selected from C₁₋₆ alkyl, C₂₋₆ alkenylene, C₂₋₆ alkynylene, C(O)NR^{c1}R^{d1}, and C(O)OR^{a2}, wherein each of said C₁₋₆ alkyl, C₂₋₆ alkenylene, and C₂₋₆ alkynylene is optionally substituted with NR^{c1}R^{d1}, C(O)R^{b1}, C(O)NR^{c1}R^{d1}, and C(O)OR^{a2}.

122. The compound or composition according to any one of embodiments 111 to 121, wherein R² is selected from C₁₋₆ alkyl and an acyl.

123. The compound or composition according to any one of embodiments 111 to 122, wherein:
R² is selected from H, C₁₋₆ alkyl, and an acyl; and
R³ is selected from C₁₋₆ alkyl, C₂₋₆ alkenylene, C₂₋₆ alkynylene, C(O)NR^{c1}R^{d1}, and C(O)OR^{a2}, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2}.

124. The compound or composition according to any one of embodiments 111 to 123, wherein R³ and OR², together with the carbon atoms to which they are attached form a tetrahydrofuran ring, which is optionally substituted with OR^{a3} or oxo; wherein R^{a3} is an acyl.

125. The compound or composition according to any one of embodiments 111 to 124, wherein R³ and OR², together with the carbon atoms to which they are attached form a tetrahydrofuran ring, which is optionally substituted with oxo.

126. The compound or composition according to any one of embodiments 111 to 125, wherein the compound of formula (IV) is selected from any one of the following compounds, or a pharmaceutically acceptable salt thereof.

Structure

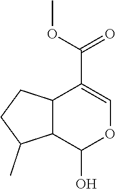

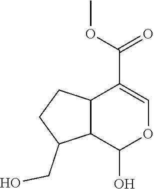 [SMILES: COC(=O)C1=COC(O)C2C(CO)CCC12]

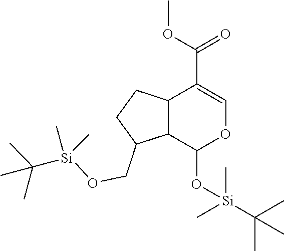 [SMILES: COC(=O)C1=COC(O[Si](C)(C)C(C)(C)C)C2C(CO[Si](C)(C)C(C)(C)C)CCC12]

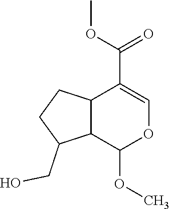 [SMILES: COC(=O)C1=COC(OC)C2C(CO)CCC12]

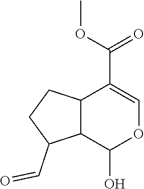 [SMILES: COC(=O)C1=COC(O)C2C(C=O)CCC12]

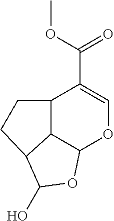 [SMILES: COC(=O)C1=COC2OC(O)C3CCC1C23]

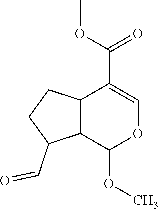 [SMILES: COC(=O)C1=COC(OC)C2C(C=O)CCC12]

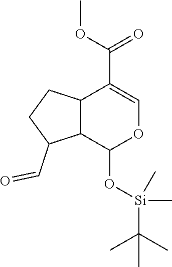 [SMILES: COC(=O)C1=COC(O[Si](C)(C)C(C)(C)C)C2C(C=O)CCC12]

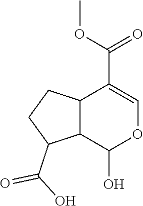 [SMILES: COC(=O)C1=COC(O)C2C(C(=O)O)CCC12]

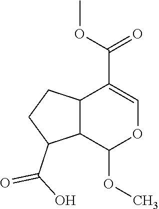 [SMILES: COC(=O)C1=COC(OC)C2C(C(=O)O)CCC12]

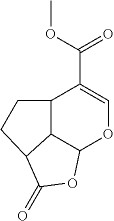 [SMILES: COC(=O)C1=COC2OC(=O)C3CCC1C23]

127. The compound or composition according to any one of embodiments 111 to 125, wherein the compound of formula (IV) is selected from any one of the following compounds, or a pharmaceutically acceptable salt thereof:

Structure

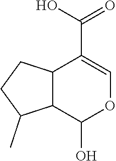

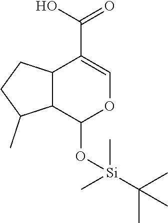 [SMILES: CC1CCC2C(C(=O)O)=COC(O[Si](C)(C)C(C)(C)C)C12]

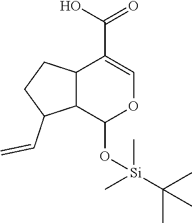 [SMILES: C=CC1CCC2C(C(=O)O)=COC(O[Si](C)(C)C(C)(C)C)C12]

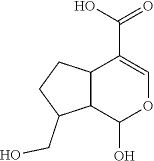 [SMILES: O=C(O)C1=COC(O)C2C(CO)CCC12]

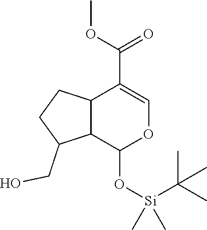 [SMILES: COC(=O)C1=COC(O[Si](C)(C)C(C)(C)C)C2C(CO)CCC12]

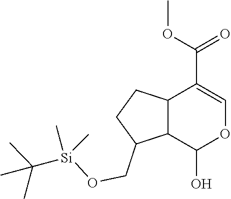 [SMILES: COC(=O)C1=COC(O)C2C(CO[Si](C)(C)C(C)(C)C)CCC12]

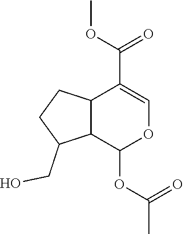 [SMILES: COC(=O)C1=COC(OC(C)=O)C2C(CO)CCC12]

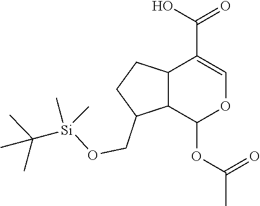 [SMILES: CC(=O)OC1OC=C(C(=O)O)C2CCC(CO[Si](C)(C)C(C)(C)C)C12]

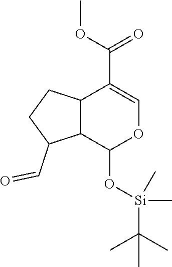 [SMILES: COC(=O)C1=COC(O[Si](C)(C)C(C)(C)C)C2C(C=O)CCC12]

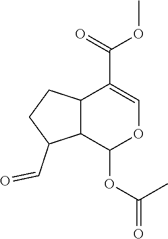 [SMILES: COC(=O)C1=COC(OC(C)=O)C2C(C=O)CCC12]

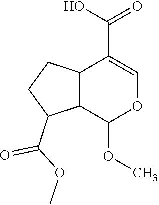 [SMILES: COC(=O)C1CCC2C(C(=O)O)=COC(OC)C12]

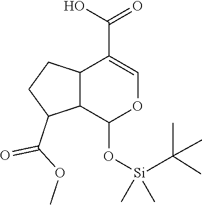 [SMILES: COC(=O)C1CCC2C(C(=O)O)=COC(O[Si](C)(C)C(C)(C)C)C12]

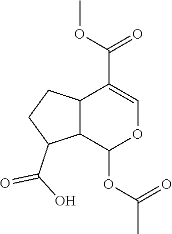 [SMILES: COC(=O)C1=COC(OC(C)=O)C2C(C(=O)O)CCC12]

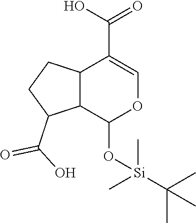 [SMILES: CC(C)(C)[Si](C)(C)OC1OC=C(C(=O)O)C2CCC(C(=O)O)C12]

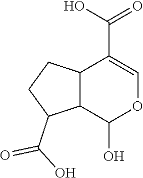 [SMILES: O=C(O)C1=COC(O)C2C(C(=O)O)CCC12]

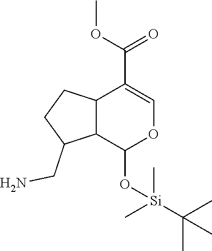 [SMILES: COC(=O)C1=COC(O[Si](C)(C)C(C)(C)C)C2C(CN)CCC12]

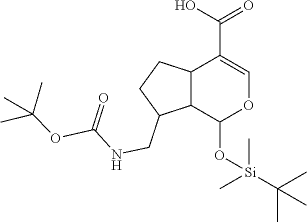 [SMILES: CC(C)(C)OC(=O)NCC1CCC2C(C(=O)O)=COC(O[Si](C)(C)C(C)(C)C)C12]

128. A compound of formula (V) or the topical composition according to any one of embodiments 1 to 22 wherein the compound of formula (I) is a compound of formula (V):

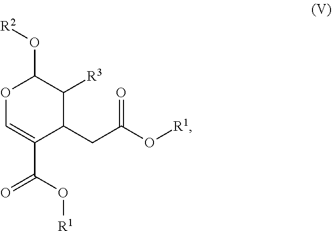

or a pharmaceutically acceptable salt thereof, wherein R¹, R², and R³ are as recited in any one of embodiments 111-125.

129. A compound of formula (VI) or the topical composition according to any one of embodiments 1 to 22 wherein the compound of formula (I) is a compound of formula (VI):

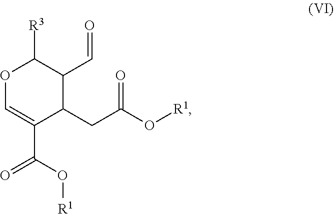

or a pharmaceutically acceptable salt thereof, wherein R¹, R², and R³ are as recited in any one of embodiments 111-125.

130. A topical sunscreen composition comprising a compound of any one of embodiments 111-129, or a pharmaceutically acceptable salt thereof, and an excipient suitable for topical administration.

131. The composition of embodiment 130, which is in the form of an aerosol spray, a cream, an emulsion, a solid, a liquid, a dispersion, a foam, an oil, a gel, a hydrogel, a lotion, a mousse, an ointment, a patch for transferring the composition onto skin, a substrate carrier comprising the topical composition, a pump spray comprising the topical composition, or a stick comprising the topical composition.

132. A non-therapeutic method of protecting skin of a subject from UV sunlight, the method comprising applying to the skin of the subject the topical composition of embodiment 130 or embodiment 131.

133. A compound or composition according to any one of embodiments 1 to 131 for use in medicine.

134. A compound or composition according to any one of embodiments 1 to 131 for use in the prevention of skin cancer, in particular melanoma.

1-46. (canceled)

47. A topical composition comprising a compound of formula (I), 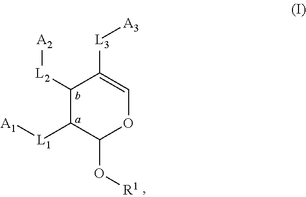
or a tautomer and/or a pharmaceutically acceptable salt thereof,
wherein A₁, A₂ and A₃ independently from each other represent,
a) a color-imparting moiety,
b) a UVA and/or UVB absorbing moiety, or
c) a C₁-C₃₀ moiety, H, hydroxyl, amino, or a halogen;
wherein R¹ represents hydrogen or a protective group hydrolysable under physiological conditions after application of the topical composition onto skin,
wherein L₁, L₂ and L₃ independently from each other represent a linker group or are absent, and
wherein L₁-A₁ and L₂-A₂ do not represent a moiety comprising an unsaturated C—C or C—N bond in alpha-position to the carbon atom marked “a” and “b”, respectively;
and an excipient suitable for topical administration.

48. The topical composition according to claim 47, wherein at least one of A₁, A₂ and A₃ is a moiety from above group a) or b).

49. The topical composition according to claim 47, wherein one or more of A₁, A₂ and A₃ represent a color-imparting moiety, wherein said moiety is:
a) an optionally substituted (hetero)aromatic moiety comprising, in combination with its optional substituents, 4 to 40 carbon atoms, or
b) an optionally substituted conjugated moiety comprising, in combination with its optional substituents, 6 to 40 carbon atoms and at least 3 conjugated C—C double bonds.

50. The topical composition according to claim 47, wherein one or more of A₁, A₂ and A₃ represents a UVA and/or UVB absorbing moiety, wherein said moiety is:
a) an optionally substituted (hetero)aromatic moiety comprising, in combination with its optional substituents, 4 to 36 carbon atoms, or
b) an optionally substituted conjugated moiety comprising, in combination with its optional substituents, 4 to 36 carbon atoms, and at least 2 conjugated C—C double bonds.

51. The topical composition according to claim 47, wherein one or more of A₁, A₂ and A₃ comprises a chromophore (dye moiety) selected from an azo group; a diazo group; a diphenylamine group; a nitroarylamine group; an azine group; an oxazine group; an acridine group; an indoline group; a sulfur dye group, a quinoid or quinone group; an anthraquinoid or anthraquinone group; a xanthene group; a naphthostyryl group; a diaryl methyl or triarylmethyl group; a benzodifuranone-based group; a formazan group; a phthalocyanine group; or a metal complex, and/or
wherein one or more of A₁, A₂ and A₃ comprises a UVA and/or UVB absorbing moiety and being selected/derived from benzophenone group, a benzotriazole group, a benzone group, salicylic acid or a salicylic acid derivative, a benzocaine group, an esculin or an esculin derivative, a ferulic acid or a ferulic acid derivative, octinoxate or an octinoxate derivative, or octocrylene or an octocrylene derivative.

52. The topical composition according to claim 47, wherein L₁ and L₂ are present and independently from each other represent optionally substituted hydrocarbon moieties each comprising, in combination with their optional substituents, 1 to 8 carbon atoms; or wherein L₁ and L₂ form a 5-, 6-, 7- or 8-membered ring.

53. The topical composition according to claim 47, wherein the C₁-C₃₀ moiety comprises 1 to 30 carbon atoms; 0 to 12 oxygen atoms; 0 to 8 nitrogen atoms; 0 to 6 sulfur atoms; and 0 to 10 halogen atoms, and/or
wherein the C₁-C₃₀ moiety is selected from a saturated or unsaturated, cyclic or acylic (hetero)alkyl comprising 1 to 30 carbon atoms; 0 to 12 oxygen atoms; 0 to 8 nitrogen atoms; 0 to 6 sulfur atoms; and 0 to 10 halogen atoms; and/or wherein the C₁-C₃₀ moiety is bound to L₁, L₂ and L₃, respectively, via a carbon atom, an oxygen atom, a nitrogen atom or a sulfur atom.

54. The topical composition according to claim 47, wherein L₁ and L₂ are present and independently from each other represent optionally substituted hydrocarbon moieties each comprising, in combination with their optional substituents, 1 to 8 carbon atoms; or wherein L₁ and L₂ form a 5-, 6-, 7- or 8-membered ring, and/or
wherein L₁ and OR₁, together with the carbon atoms to which they are attached, form a 5- or 6-membered lactone, and/or
wherein R₁ represents hydrogen or a C₁₋₆ acyl.

55. The topical composition according to claim 47, wherein the compound of formula (I) is a compound of formula (II), 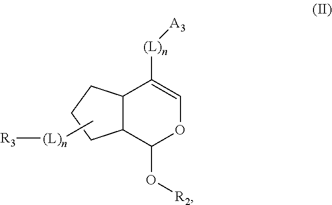
or a tautomer and/or a pharmaceutically acceptable salt thereof,
wherein:
R¹ and R³ independently from each other represent H; a C₁-C₃₀ moiety comprising 1 to 30 carbon atoms; 0 to 12 oxygen atoms; 0 to 8 nitrogen atoms; 0 to 6 sulfur atoms; and 0 to 10 halogen atoms; —SO₃H; —NO₂; —NH₂; —OH; —SH; or halogen, and, in case of R³, oxo;
R² is selected from H or a protective group hydrolysable under physiological conditions after application in a topical composition onto skin; and
optionally wherein (L)ₙR³ and OR², together with the carbon atoms to which they are attached, form a tetrahydrofuran ring which is optionally substituted with OR^{a1} or oxo;
n is 0 or is an integer selected from 1 to 10, wherein, if n=0, (L)ₙ represents a bond;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl.

56. The compound or composition according to claim 55, wherein R¹ and/or R³ represent said C₁-C₃₀ moiety which further is a color-imparting moiety, or wherein R¹ and/or R³ represent said C₁-C₃₀ moiety which further is a UVA and/or UVB absorbing moiety.

57. The composition according to claim 55, wherein the compound of formula (II) is a compound of formula (III),

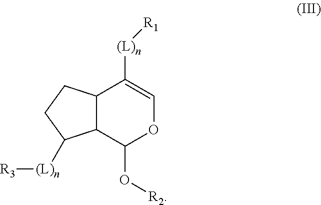

58. The composition according to claim 55, wherein the C₁-C₃₀ moiety of R³ is attached to (L)ₙ or the cyclopentyl ring of formula (II) via a carbon atom, an oxygen atom, a nitrogen atom or a sulfur atom, and/or
wherein the C₁-C₃₀ moiety of R¹ is attached to (L)ₙ or to the 3,4-dihydro-2H-pyran ring of formula (II) via a carbon atom, an oxygen atom, a nitrogen atom or a sulfur atom.

59. The composition according to claim 55, wherein (L)ₙR³ represents R^{a}, O—R^{a}, SR^{a}, (CH₂)₁₋₄—O—R^{a}, C(O)R^{a}, CO₂R^{a}, O—C(O)R^{a}, NR^{a}₂, NHR^{a}, NHC(O)R^{a}, NR^{a}C(O)R^{a}, or oxo, wherein R^{a} independently from each other represents a C₁-C₁₈ moiety comprising 1 to 30 carbon atoms; 0 to 12 oxygen atoms; 0 to 8 nitrogen atoms; 0 to 6 sulfur atoms; and 0 to 10 halogen atoms.

60. The composition according to claim 56, wherein (L)ₙR¹ represents CO₂R^{a}, C(O)R^{a} or R^{a}, wherein R^{a} represents optionally substituted phenyl.

61. The topical composition according to claim 47, wherein the compound of formula (I) is a compound of formula (VII): 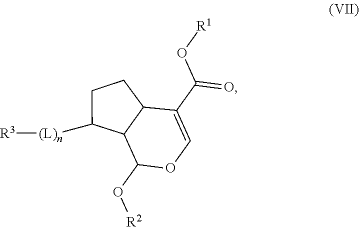
or a pharmaceutically acceptable salt thereof, wherein:
R¹ is selected from H and C₁₋₆ alkyl;
R² is selected from H and an acyl;
n is 0 or an integer selected from 1 to 10, wherein, if n=0, (L)ₙ represents a bond;
each L is independently selected from C(═O), N(R^{N}), (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is a dye moiety,
or
wherein the compound of formula (I) is a compound of formula (VIII): 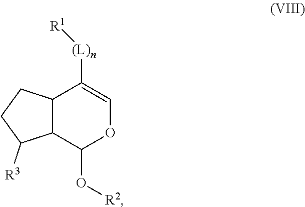
or a pharmaceutically acceptable salt thereof, wherein:
R¹ is a dye moiety;
R² is selected from H and an acyl; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄ haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

62. The topical composition according to claim 47, wherein the compound of formula (I) is a compound of formula (IX): 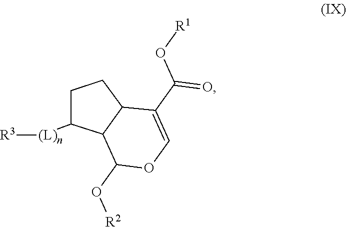
or a pharmaceutically acceptable salt thereof, wherein:
R¹ is selected from H and C₁₋₆ alkyl;
R² is selected from H and an acyl;
n is 0 or an integer selected from 1 to 10, wherein, if n=0, (L)ₙ represents a bond;
each L is independently selected from C(═O), N(R^{N}), O, (—C₁₋₃ alkylene-O—)ₓ, (—O—C₁₋₃ alkylene-)ₓ, and —C₁₋₃ alkylene-, wherein each x is independently an integer from 1 to 10 and each C₁₋₃ alkylene is optionally substituted with 1, 2, or 3 substituents independently selected from OH, NO₂, CN, halo, amino, and carboxy;
each R^{N} is independently selected from H and C₁₋₃ alkyl; and
R³ is a UV-absorbing moiety,
or
wherein the compound of formula (I) is a compound of formula (X): 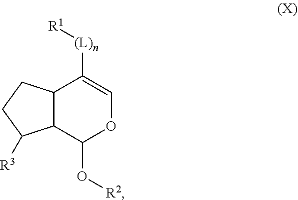
or a pharmaceutically acceptable salt thereof, wherein:
R¹ is a UV-absorbing moiety;
R² is selected from H and an acyl; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄ haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

63. The topical composition according to claim 47 wherein the compound of formula (I) is a compound of formula (IV): 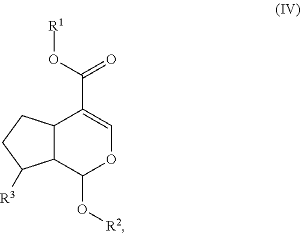
or a pharmaceutically acceptable salt thereof, wherein:
R¹ is selected from H and a carboxylic acid-protecting group;
R² is as defined in claim 9; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄ haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

64. A non-therapeutic method of semi-permanently tattooing skin of a subject or a non-therapeutic method of protecting skin of a subject from UV sunlight, the method comprising applying to the skin of the subject the topical composition of claim 47.

65. A compound of formula (IV): 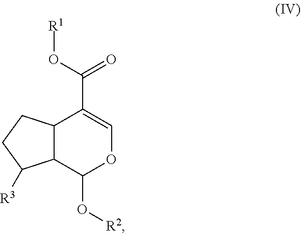
or a pharmaceutically acceptable salt thereof, wherein:
R¹ is selected from H and a carboxylic acid-protecting group;
R² is selected from H or a protective group hydrolysable under physiological conditions after application in a topical composition onto skin; and
R³ is selected from C₁₋₆ alkyl, C₂₋₆ alkenylene, C₂₋₆ alkynylene, C(O)R^{b1}, C(O)NR^{c1}R^{d1}, C(O)OR^{a2} and oxo, wherein each of said C₁₋₆ alkyl, C₂₋₆ alkenylene, and C₂₋₆ alkynylene is optionally substituted with 1 or 2 substituents independently selected from OR^{a1}, NR^{c1}R^{d1}, C(O)R^{b1}, C(O)NR^{c1}R^{d1}, and C(O)OR^{a2};
or wherein R³ and OR², together with the carbon atoms to which they are attached form a tetrahydrofuran ring, which is optionally substituted with OR^{a1} or oxo;
each R^{a1} is independently selected from H, C₁₋₆ alkyl, C₁₋₄ haloalkyl, C₂₋₆ alkenyl, C₂₋₆ alkynyl, and a hydroxyl-protecting group;
each R^{a2} is independently selected from H, C₁₋₆ alkyl, C₁₋₄ haloalkyl, C₂₋₆ alkenyl, C₂₋₆ alkynyl, and a carboxyl-protecting group;
each R^{c1} and R^{d1} is independently selected from H, C₁₋₆ alkyl, C₁₋₄ haloalkyl, C₂₋₆ alkenyl, C₂₋₆ alkynyl, and an amino-protecting group; and
each R^{b1} is independently selected from H, C₁₋₆ alkyl, C₁₋₄ haloalkyl, C₂₋₆ alkenyl, and C₂₋₆ alkynyl.

66. A compound according to claim 65, wherein the compound of formula (IV) is selected from any one of the following compounds, or a pharmaceutically acceptable salt thereof:
Structure 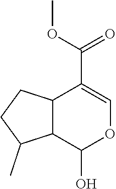 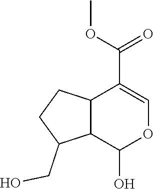 [SMILES: COC(=O)C1=COC(O)C2C(CO)CCC12] 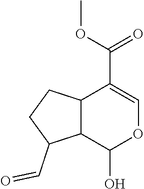 [SMILES: COC(=O)C1=COC(O)C2C(C=O)CCC12] 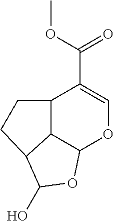 [SMILES: COC(=O)C1=COC2OC(O)C3CCC1C23] 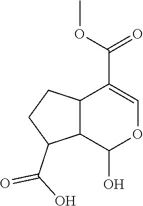 [SMILES: COC(=O)C1=COC(O)C2C(C(=O)O)CCC12] 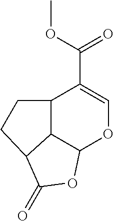 [SMILES: COC(=O)C1=COC2OC(=O)C3CCC1C23]
